(19) United States
(12) Patent Application Publication
(10) Pub. No.: US 2023/0218765 A1
(43) Pub. Date: Jul. 13, 2023

(54) SMALL MOLECULE DEGRADERS OF SHP2 PROTEIN

(71) Applicant: THE REGENTS OF THE UNIVERSITY OF MICHIGAN, Ann Arbor, MI (US)

(72) Inventors: Shaomeng Wang, Superior Township, MI (US); Mingliang Wang, Ann Arbor, MI (US); Jianfeng Lu, Ann Arbor, MI (US); Mi Wang, Ann Arbor, MI (US)

(21) Appl. No.: 17/924,794

(22) PCT Filed: May 19, 2021
(86) PCT No.: PCT/US2021/033166

§ 371 Date: Nov. 11, 2022

Related U.S. Application Data
(60) Provisional application No. 63/026,900, filed on May 19, 2020.

(51) Int. Cl. A61K 47/54; A61K 47/55; A61P 35/00

(52) U.S. Cl. CPC A61K 47/545; A61K 47/55; A61P 35/00

(57) ABSTRACT

The present disclosure provides compounds represented by Formula I: and the salts or solvates thereof, wherein R1, R3, L, Y, and B1 are as defined in the specification. Compounds having Formula I are SHP2 protein degraders useful for the treatment of cancer and other diseases.

### BACKGROUND OF THE INVENTION

### Field of the Invention

The present disclosure provides heterobifunctional small molecules as Src homology 2 domain-containing phosphatase (SHP2) protein degraders. SHP2 degraders are useful for the treatment of a variety of diseases including cancer.

### Background

Src homology 2 domain-containing phosphatase (SHP2) is a protein tyrosine phosphatase. Mutations of SHP2 are prevalent in Noonan syndrome (50%) and LEOPARD syndrome (80%) (Tartaglia et al., Nat. Genet. 2001, 29, 465-468; Kontaridis et al., J. Biol. Chem. 2006, 281, 6785-6792) and activated mutations of SHP2 have also been identified in juvenile myelomonocytic leukemia (JMML, 35%), myelodysplastic syndrome (10%), B-cell acute lymphoblastic leukemia (7%) and acute myeloid leukemia (AML, 4%). Tartaglia et al., Nat. Genet. 2003, 34, 148-150. Somatic activating mutations in SHP2 have been associated with several types of solid tumors, including lung adenocarcinoma, colon cancer, neuroblastoma, glioblastoma, melanoma, hepatocellular carcinoma, prostate cancer and breast cancer. See, e.g., Bentires-Alj et al., Cancer. Res. 2004, 64, 8816-8820.

Accumulated evidence demonstrates that in cancer cells, SHP2 is involved in multiple signaling processes, such as RAS-ERK, JAK-STAT, PI3K-AKT, NF-κB and mTOR pathways. See, e.g., Agazie et al., Mol. Cell Biol. 2003, 23, 7875-7886. In the RAS-ERK pathway, SHP2 acts as a positive regulator at upstream to promote RAS-RAF-ERK kinase cascade signaling transduction. SHP2 inhibition thus leads to dephosphorylation of ERK and suppression of the pro-oncogenic function of RAS-RAF-ERK pathway, resulting in cell growth inhibition and apoptosis induction in cancer cells. Bunda et al., Nat. Commun. 2015, 6, 8859. SHP2 also participates in the programmed cell death pathway (PD-1/PD-L1) and inhibits T cell activation, thus contributing to immune evasion. See, e.g., Chemnitz et al., J. Immunol. 2004, 173, 945-954. In sum, SHP2 is a very attractive cancer therapeutic target.

Due to the highly conserved and positively charged nature of its protein-tyrosine phosphatase (PTP) catalytic site, SHP2 has proved to be a difficult target in the discovery of small-molecule inhibitors. Scott et al., Curr. Pharm. Des. 2010, 16, 1843-1862; Butterworth et al., Future Med. Chem. 2014, 6, 1423-1437. Previously reported SHP2 inhibitors have not shown satisfactory selectivity and/or cellular activity, and this has prevented their development as useful therapeutic agents. See, e.g., Chen et al., Mol. Pharmacol. 2006, 70, 562-570. A breakthrough in this field was the discovery of SHP099, a potent and allosteric SHP2 inhibitor, which was shown to selectively block SHP2 phosphatase activity and inhibit cancer cell growth in vitro and tumor growth in xenograft models in mice. Chen et al., Nature 2016, 535, 148-152; Garcia Fortanet et al., J. Med. Chem. 2016, 59, 7773-7782. Subsequently, additional allosteric SHP2 inhibitors including SHP389 were reported and several of them have been advanced into clinical development for the treatment of human cancers. Xie et al.,J. Med. Chem. 2017, 60, 10205-10219; Bagdanoff et al., J. Med. Chem. 2019, 62, 1781-1792; Sarver et al., J. Med. Chem. 2019, 62, 1793-1802.

Allosteric SHP2 inhibitors have been shown to be effective in preclinical models of Kirsten rat sarcoma (KRAS)-mutant human cancer. Depletion of SHP2 protein may also provide an alternative and perhaps even more effective strategy for inhibition of the SHP2 activity. Mainardi et al. demonstrated that SHP2 inactivation by CRISPR-Cas9 induces senescence and impairs tumor growth in xenograft models of KRAS-mutant tumors. Mainardi et al., Nat. Med. 2018, 24, 961-967. Ruess et al. disclosed that knockout of the PTPN11 gene, which encodes SHP2, in KRAS-mutant human ductal adenocarcinoma (PDAC) cells results in reduced in cell proliferation and PTPN11-knockout cells are uniquely susceptible to mitogen-activated protein kinase (MEK) inhibitors. Ruess et al., Nat. Med. 2018, 24, 954-960. Such findings provide evidence that depletion of SHP2 protein in tumor cells could be an effective therapeutic strategy for human cancers, particularly those carrying a KRAS-mutation.

The Proteolysis Targeting Chimera (PROTAC) strategy has gained momentum with its promise in the discovery and development of completely new types of small molecule therapeutics by inducing targeted protein degradation. Raina et al., Proc Natl Acad Sci USA. 2016,113, 7124-7129; Zhou et al., J. Med. Chem. 2018, 61, 462-481.

A PROTAC molecule is a heterobifunctional small molecule containing one ligand, which binds to the target protein of interest, and a second ligand for an E3 ligase system, tethered together by a chemical linker. Bondeson, D. P.; Crews, C. M. Targeted Protein Degradation by Small Molecules. Annu Rev Pharmacol Toxicol. 2017, 57, 107-123.

There is a need in the art for SHP2 degraders to treat cancer and other diseases.

### BRIEF SUMMARY OF THE INVENTION

In one aspect, the present disclosure provides heterobifunctional small molecules represented by Formula I, below, and the pharmaceutically acceptable salts and solvates, e.g., hydrates, thereof. These compounds, and the salts and solvates thereof are collectively referred to herein as “Compounds of the Disclosure.” Compounds of the Disclosure are SHP2 degraders and are thus useful in treating diseases or conditions wherein degradation of the SHP2 protein provides a therapeutic benefit to a subject.

In another aspect, the present disclosure provides methods of treating a condition or disease by administering a therapeutically effective amount of a Compound of the Disclosure to a subject, e.g., a human cancer patient, in need thereof. The disease or condition treatable by degradation of SHP2 is, for example, a cancer, e.g., prostate cancer, e.g., metastatic castration-resistant prostate cancer.

In another aspect, the present disclosure provides a method of degrading, e.g., reducing the level of, of SHP2 protein in a subject in need thereof, comprising administering to the individual an effective amount of at least one Compound of the Disclosure.

In another aspect, the present disclosure provides a pharmaceutical composition comprising a Compound of the Disclosure and an excipient and/or pharmaceutically acceptable carrier.

In another aspect, the present disclosure provides a composition comprising a Compound of the Disclosure and an excipient and/or pharmaceutically acceptable carrier for use treating diseases or conditions wherein degradation of the SHP2 protein provides a benefit, e.g., cancer.

In another aspect, the present disclosure provides a composition comprising: (a) a Compound of the Disclosure; (b) a second therapeutically active agent; and (c) optionally an excipient and/or pharmaceutically acceptable carrier.

In another aspect, the present disclosure provides a Compound of the Disclosure for use in treatment of a disease or condition of interest, e.g., cancer.

In another aspect, the present disclosure provides a use of a Compound of the Disclosure for the manufacture of a medicament for treating a disease or condition of interest, e.g., cancer.

In another aspect, the present disclosure provides a kit comprising a Compound of the Disclosure, and, optionally, a packaged composition comprising a second therapeutic agent useful in the treatment of a disease or condition of interest, and a package insert containing directions for use in the treatment of a disease or condition, e.g., cancer.

In another aspect, the present disclosure provides methods of preparing Compounds of the Disclosure.

Additional embodiments and advantages of the disclosure will be set forth, in part, in the description that follows, and will flow from the description, or can be learned by practice of the disclosure. The embodiments and advantages of the disclosure will be realized and attained by means of the elements and combinations particularly pointed out in the appended claims. It is to be understood that both the foregoing summary and the following detailed description are exemplary and explanatory only, and are not restrictive of the invention as claimed.

### BRIEF DESCRIPTION OF DRAWINGS

FIG. 1 is line graph showing the dose response curves of compound 4, compound 5, and SPH099 in the SHP2 allosteric inhibition assay.

FIG. 2A is a Western blot analysis showing the degradation activity of compound 26 (SHP2-D26) in KYSE520 cells at the concentrations indicated after 12 h.

FIG. 2B is a line graph showing the DC₅₀ of compound 26 (SHP2-D26) in KYSE520 cells after 12 h.

FIG. 2C is a Western blot analysis showing the degradation activity of compound 26 (SHP2-D26) in MV4;11 cells at the concentrations indicated after 12 h.

FIG. 2D is line graph showing the DC₅₀ of compound 26 (SHP2-D26) in MV4;11 cells after 12 h.

FIG. 2E is a Western blot analysis showing the degradation activity of compound 26 (SHP2-D26) in KYSE520 cells at 100 nM at the time points indicated.

FIG. 2F is a Western blot analysis showing the degradation activity of compound 26 (SHP2-D26) in MV4;11 cells at 100 nM at the time points indicated.

FIG. 2G is a Western blot analysis showing the degradation activity of compound 26 (SHP2-D26) in KYSE520 cells in combination with other compounds.

FIG. 3A is a Western blot analysis showing the p-ERK activity of compound 26 (SHP2-D26) in KYSE520 cells.

FIG. 3B is a Western blot analysis showing the p-ERK activity of compound 26 (SHP2-D26) in MV4;11 cells.

FIG. 4A is a line graph showing the cell growth inhibition of compounds 4, 5, and 26 (SHP2-D26) in KYSE520 cells.

FIG. 4B is a line graph showing the cell growth inhibition of compounds 4, 5, and 26 (SHP2-D26) in MV4;11 cells.

### DETAILED DESCRIPTION OF THE INVENTION

### I. Compounds of the Disclosure

Compounds of the Disclosure are heterobifunctional SHP2 degraders. In one embodiment, Compounds of the Disclosure are compounds of Formula I:

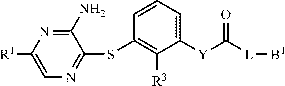

or a pharmaceutically acceptable salt or solvate thereof, wherein

R¹ is selected from the group consisting of:

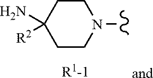

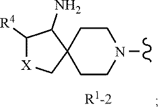

R² is selected from the group consisting of C₁-₄ alkyl and C₃-C₆ cycloalkyl;
R³ is selected from the group consisting of halo and C₁-C₃ haloalkyl;
R⁴ is selected from the group consisting of hydrogen and C₁-₄ alkyl;
X is selected from the group consisting of —O— and —CH₂—;
Y is selected from the group consisting of —NH— and

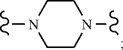

L is -J¹-J²-J³-J⁴-;
J¹ is —(CH₂)ₘ—;
m is 1, 2, 3, or 4;
J² is selected from the group consisting of:

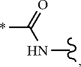

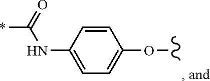

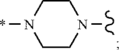

wherein the bond marked with an “*” is attached to J¹; or
J² is absent, i.e., J² is a bond;
J³ is selected from the group consisting of —(CH₂)ₙ— and —(CH₂CH₂O)ₒ—;
n is 1, 2, 3, 4, 5, 6, 7, 8, 9, 10, 11, 12, 13, 14, 15, or 16;
o is 1, 2, 3, 4, 5, 6, 7, or 8;
J⁴ is selected from the group consisting of —(CH₂)ₚ—, —O—, —N(H)—, —C═C—,

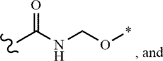

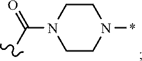

wherein the bond marked with an “*” is attached to B¹;
p is 0, 1, 2, or 3;
with the proviso that J⁴ is —(CH₂)ₚ— and p is 1, 2, or 3, when B¹ is B¹-1;
B¹ is selected from the group consisting of:

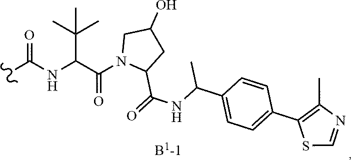

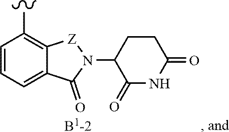

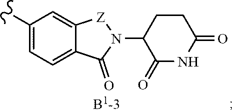

and
Z is selected from the group consisting of —CH₂ and —C(═O)—.

In another embodiment, Compounds of the Disclosure are compounds of Formula I, or a pharmaceutically acceptable salt or solvate thereof, wherein R¹ is R¹-1.

In another embodiment, Compounds of the Disclosure are compounds of Formula I, or a pharmaceutically acceptable salt or solvate thereof, wherein R² is selected from the group consisting of methyl and ethyl.

In another embodiment, Compounds of the Disclosure are compounds of Formula I, or a pharmaceutically acceptable salt or solvate thereof, wherein R¹ is R¹-2. In another embodiment, R⁴ is hydrogen. In another embodiment, R⁴ is methyl. In another embodiment, X is —O—. In another embodiment, X is —CH₂—.

In another embodiment, Compounds of the Disclosure are compounds of Formula I, or a pharmaceutically acceptable salt or solvate thereof, wherein R³ is —Cl.

In another embodiment, Compounds of the Disclosure are compounds of Formula I, or a pharmaceutically acceptable salt or solvate thereof, wherein Y is —NH—.

In another embodiment, Compounds of the Disclosure are compounds of Formula I, or a pharmaceutically acceptable salt or solvate thereof, wherein Y is:

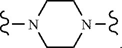

In another embodiment, Compounds of the Disclosure are compounds of Formula I, or a pharmaceutically acceptable salt or solvate thereof, wherein B¹ is:

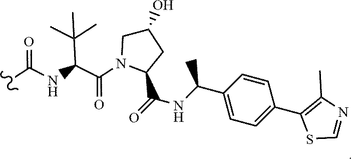

In another embodiment, Compounds of the Disclosure are compounds of Formula I, or a pharmaceutically acceptable salt or solvate thereof, wherein B¹ is B¹-2. In another embodiment, Z is —C(═O)—.

In another embodiment, Compounds of the Disclosure are compounds of Formula I, or a pharmaceutically acceptable salt or solvate thereof, wherein B¹ is B¹-3. In another embodiment, Z is —C(═O)—.

In another embodiment, Compounds of the Disclosure are compounds of Formula I, or a pharmaceutically acceptable salt or solvate thereof, wherein:

L is selected from the group consisting of:

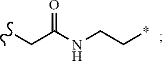

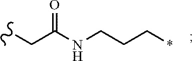

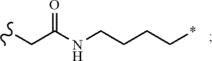

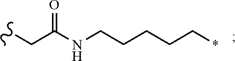

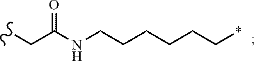

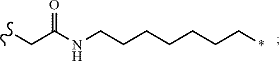

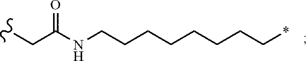

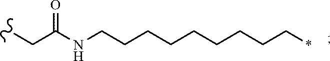

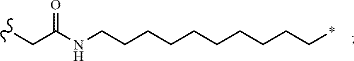

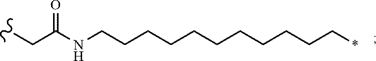

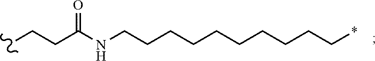

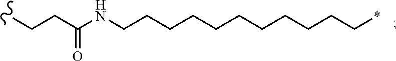

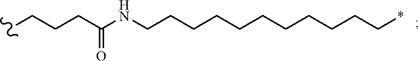

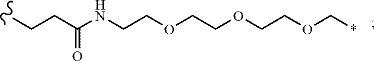

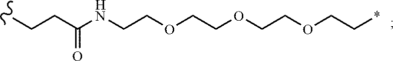

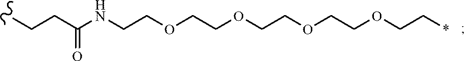

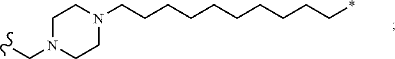

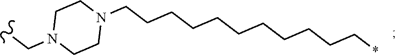

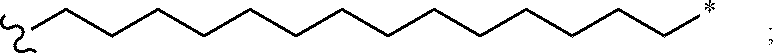

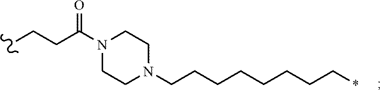

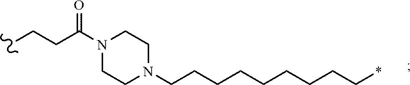

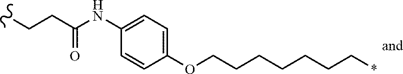

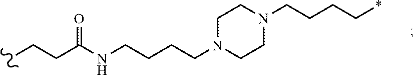

wherein the bond marked with an “*” is attached to B¹; and B¹ is B¹-1.

In another embodiment, Compounds of the Disclosure are compounds of Formula I, or a pharmaceutically acceptable salt or solvate thereof, wherein:

L is selected from the group consisting of:

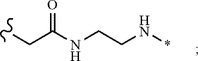

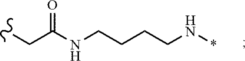

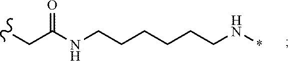

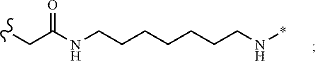

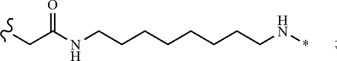

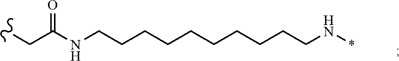

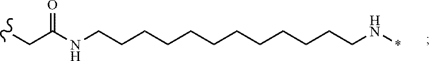

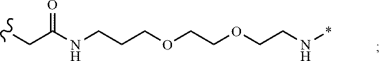

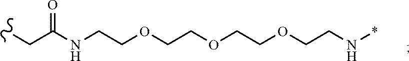

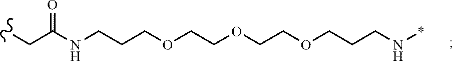

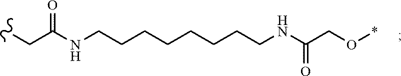

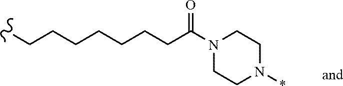

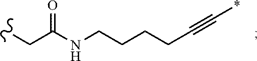

wherein the bond marked with an “*” is attached to B¹; and B¹ is selected from the group consisting of B¹-2 and B¹-3.

In another embodiment, Compounds of the Disclosure are any one or more of the compounds of Table 1, or a pharmaceutically acceptable salt or solvate thereof.

TABLE 1

| Compound | Structure |
|---|---|
| 7 | 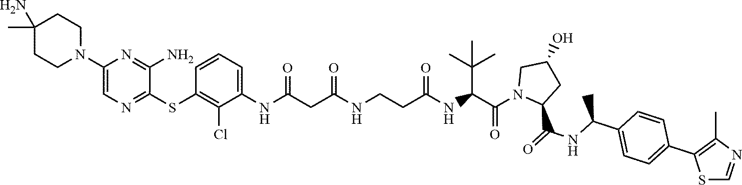 [SMILES: Cc1ncsc1-c1ccc([C@H](C)NC(=O)[C@@H]2C[C@@H](O)CN2C(=O)[C@@H](NC(=O)CCNC(=O)CC(=O)Nc2cccc(Sc3ncc(N4CCC(C)(N)CC4)nc3N)c2Cl)C(C)(C)C)cc1] |
| 8 | 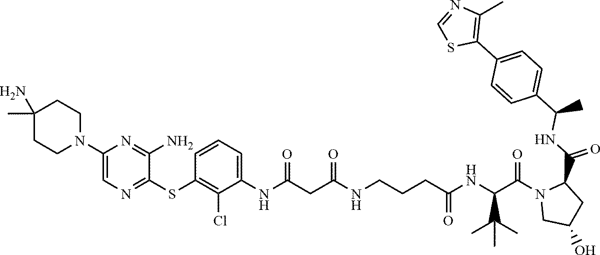 [SMILES: Cc1ncsc1-c1ccc([C@@H](C)NC(=O)[C@H]2C[C@H](O)CN2C(=O)[C@H](NC(=O)CCCNC(=O)CC(=O)Nc2cccc(Sc3ncc(N4CCC(C)(N)CC4)nc3N)c2Cl)C(C)(C)C)cc1] |
| 9 | 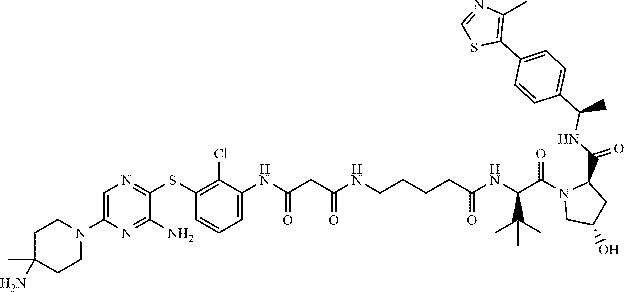 [SMILES: Cc1ncsc1-c1ccc([C@@H](C)NC(=O)[C@H]2C[C@H](O)CN2C(=O)[C@H](NC(=O)CCCCNC(=O)CC(=O)Nc2cccc(Sc3ncc(N4CCC(C)(N)CC4)nc3N)c2Cl)C(C)(C)C)cc1] |
| 10 | 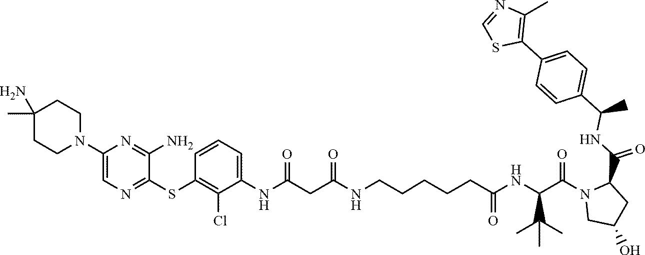 [SMILES: Cc1ncsc1-c1ccc([C@@H](C)NC(=O)[C@H]2C[C@H](O)CN2C(=O)[C@H](NC(=O)CCCCCNC(=O)CC(=O)Nc2cccc(Sc3ncc(N4CCC(C)(N)CC4)nc3N)c2Cl)C(C)(C)C)cc1] |
| 11 | 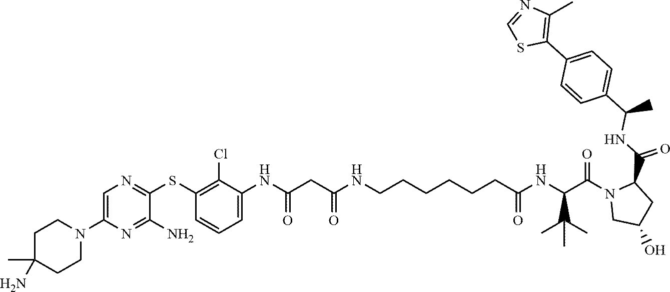 [SMILES: Cc1ncsc1-c1ccc([C@@H](C)NC(=O)[C@H]2C[C@H](O)CN2C(=O)[C@H](NC(=O)CCCCCCNC(=O)CC(=O)Nc2cccc(Sc3ncc(N4CCC(C)(N)CC4)nc3N)c2Cl)C(C)(C)C)cc1] |
| 12 | 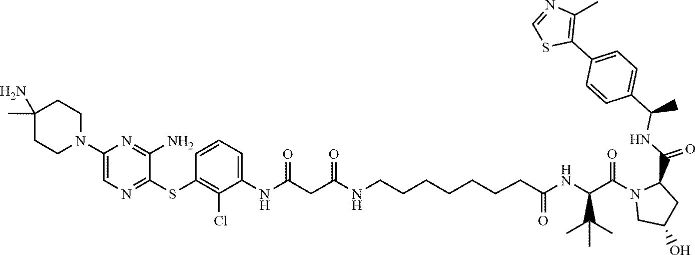 [SMILES: Cc1ncsc1-c1ccc([C@@H](C)NC(=O)[C@H]2C[C@H](O)CN2C(=O)[C@H](NC(=O)CCCCCCCNC(=O)CC(=O)Nc2cccc(Sc3ncc(N4CCC(C)(N)CC4)nc3N)c2Cl)C(C)(C)C)cc1] |
| 13 | 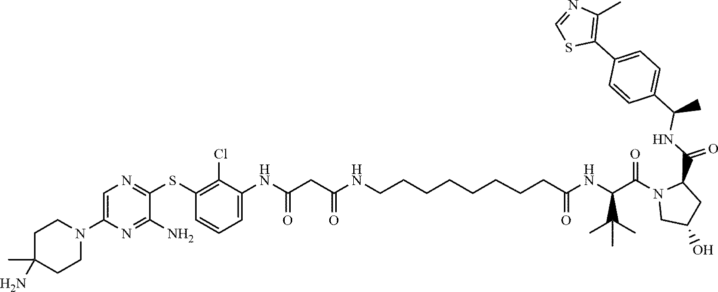 [SMILES: Cc1ncsc1-c1ccc([C@@H](C)NC(=O)[C@H]2C[C@H](O)CN2C(=O)[C@H](NC(=O)CCCCCCCCNC(=O)CC(=O)Nc2cccc(Sc3ncc(N4CCC(C)(N)CC4)nc3N)c2Cl)C(C)(C)C)cc1] |
| 14 | 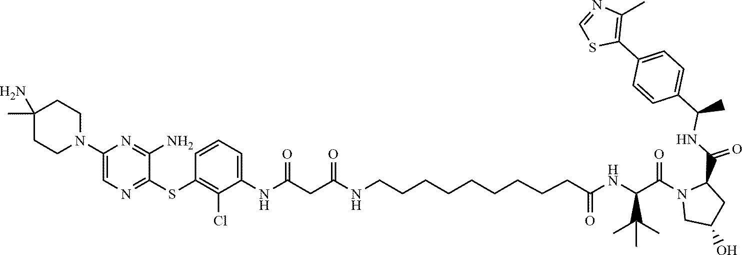 [SMILES: Cc1ncsc1-c1ccc([C@@H](C)NC(=O)[C@H]2C[C@H](O)CN2C(=O)[C@H](NC(=O)CCCCCCCCCNC(=O)CC(=O)Nc2cccc(Sc3ncc(N4CCC(C)(N)CC4)nc3N)c2Cl)C(C)(C)C)cc1] |
| 15 | 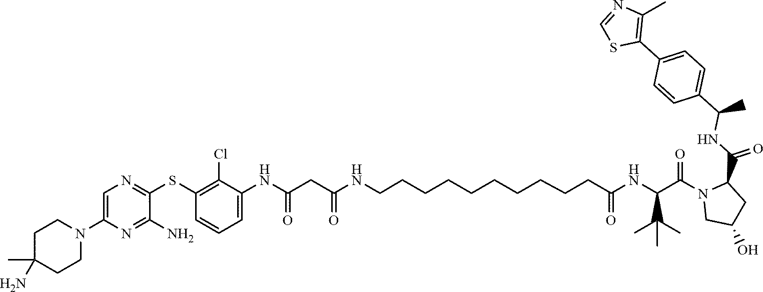 [SMILES: Cc1ncsc1-c1ccc([C@@H](C)NC(=O)[C@H]2C[C@H](O)CN2C(=O)[C@H](NC(=O)CCCCCCCCCCNC(=O)CC(=O)Nc2cccc(Sc3ncc(N4CCC(C)(N)CC4)nc3N)c2Cl)C(C)(C)C)cc1] |
| 16 | 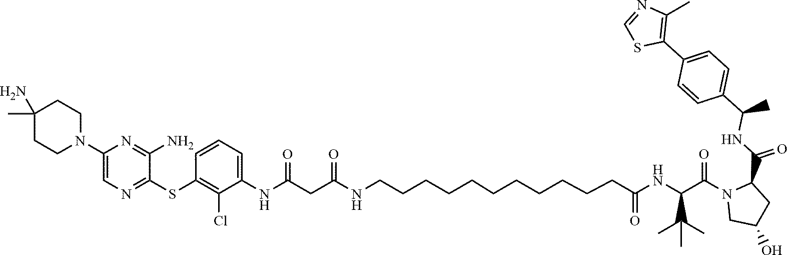 [SMILES: Cc1ncsc1-c1ccc([C@@H](C)NC(=O)[C@H]2C[C@H](O)CN2C(=O)[C@H](NC(=O)CCCCCCCCCCCNC(=O)CC(=O)Nc2cccc(Sc3ncc(N4CCC(C)(N)CC4)nc3N)c2Cl)C(C)(C)C)cc1] |
| 17 | 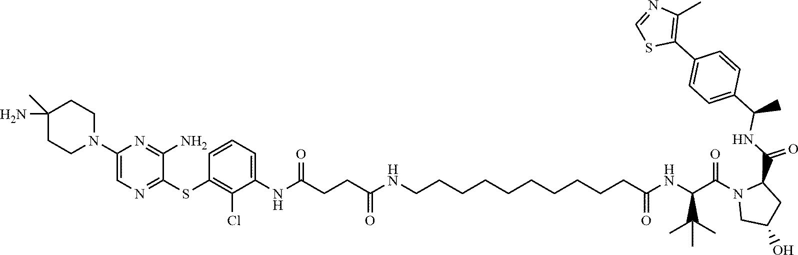 [SMILES: Cc1ncsc1-c1ccc([C@@H](C)NC(=O)[C@H]2C[C@H](O)CN2C(=O)[C@H](NC(=O)CCCCCCCCCCNC(=O)CCC(=O)Nc2cccc(Sc3ncc(N4CCC(C)(N)CC4)nc3N)c2Cl)C(C)(C)C)cc1] |
| 18 | 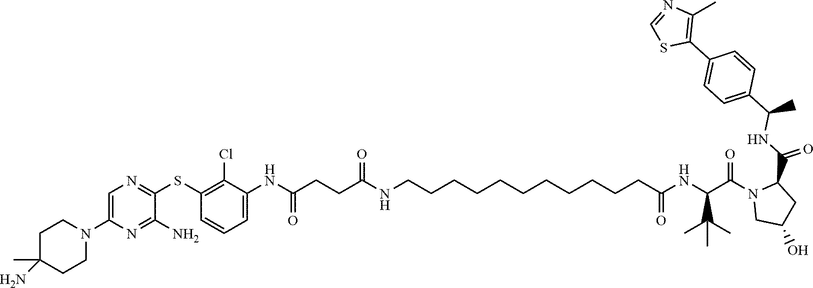 [SMILES: Cc1ncsc1-c1ccc([C@@H](C)NC(=O)[C@H]2C[C@H](O)CN2C(=O)[C@H](NC(=O)CCCCCCCCCCCNC(=O)CCC(=O)Nc2cccc(Sc3ncc(N4CCC(C)(N)CC4)nc3N)c2Cl)C(C)(C)C)cc1] |
| 19 | 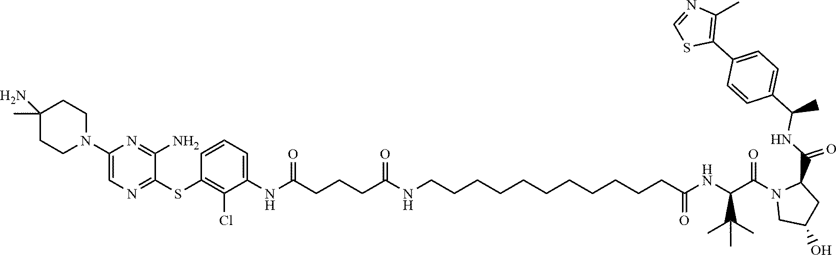 [SMILES: Cc1ncsc1-c1ccc([C@@H](C)NC(=O)[C@H]2C[C@H](O)CN2C(=O)[C@H](NC(=O)CCCCCCCCCCCNC(=O)CCCC(=O)Nc2cccc(Sc3ncc(N4CCC(C)(N)CC4)nc3N)c2Cl)C(C)(C)C)cc1] |
| 20 | 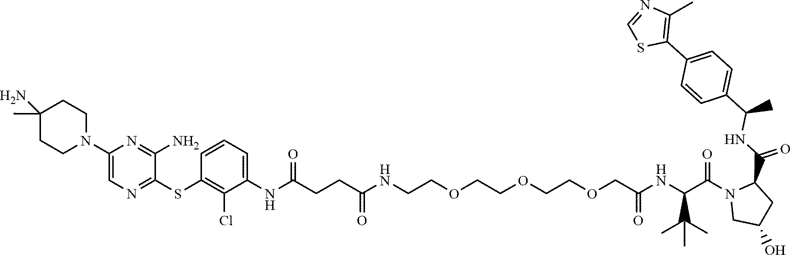 [SMILES: Cc1ncsc1-c1ccc([C@@H](C)NC(=O)[C@H]2C[C@H](O)CN2C(=O)[C@H](NC(=O)COCCOCCOCCNC(=O)CCC(=O)Nc2cccc(Sc3ncc(N4CCC(C)(N)CC4)nc3N)c2Cl)C(C)(C)C)cc1] |
| 21 | 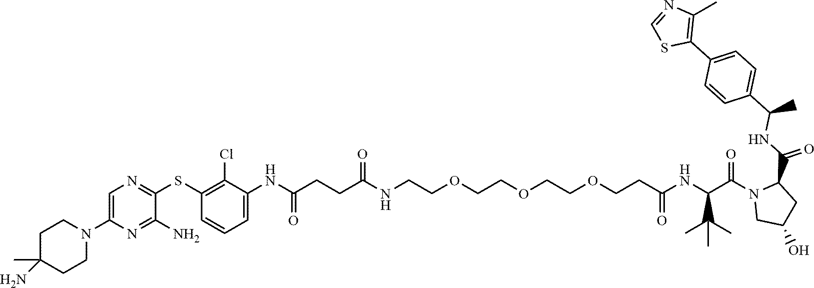 [SMILES: Cc1ncsc1-c1ccc([C@@H](C)NC(=O)[C@H]2C[C@H](O)CN2C(=O)[C@H](NC(=O)CCOCCOCCOCCNC(=O)CCC(=O)Nc2cccc(Sc3ncc(N4CCC(C)(N)CC4)nc3N)c2Cl)C(C)(C)C)cc1] |
| 22 | 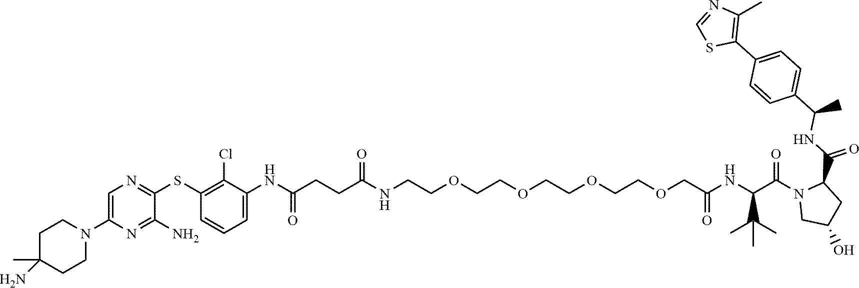 [SMILES: Cc1ncsc1-c1ccc([C@@H](C)NC(=O)[C@H]2C[C@H](O)CN2C(=O)[C@H](NC(=O)COCCOCCOCCOCCNC(=O)CCC(=O)Nc2cccc(Sc3ncc(N4CCC(C)(N)CC4)nc3N)c2Cl)C(C)(C)C)cc1] |
| 23 | 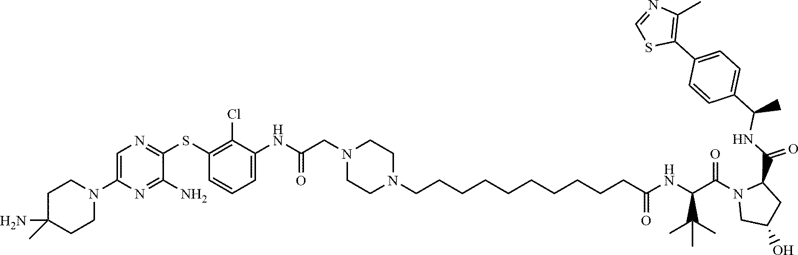 [SMILES: Cc1ncsc1-c1ccc([C@@H](C)NC(=O)[C@H]2C[C@H](O)CN2C(=O)[C@H](NC(=O)CCCCCCCCCCN2CCN(CC(=O)Nc3cccc(Sc4ncc(N5CCC(C)(N)CC5)nc4N)c3Cl)CC2)C(C)(C)C)cc1] |
| 24 | 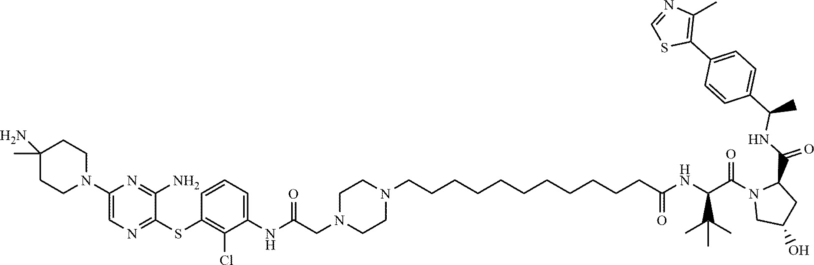 [SMILES: Cc1ncsc1-c1ccc([C@@H](C)NC(=O)[C@H]2C[C@H](O)CN2C(=O)[C@H](NC(=O)CCCCCCCCCCCN2CCN(CC(=O)Nc3cccc(Sc4ncc(N5CCC(C)(N)CC5)nc4N)c3Cl)CC2)C(C)(C)C)cc1] |
| 25 | 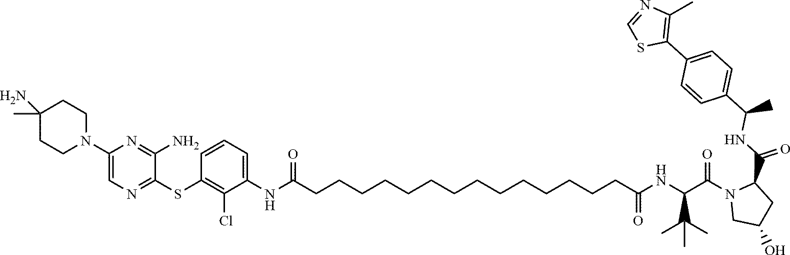 [SMILES: Cc1ncsc1-c1ccc([C@@H](C)NC(=O)[C@H]2C[C@H](O)CN2C(=O)[C@H](NC(=O)CCCCCCCCCCCCCCC(=O)Nc2cccc(Sc3ncc(N4CCC(C)(N)CC4)nc3N)c2Cl)C(C)(C)C)cc1] |
| 26 | 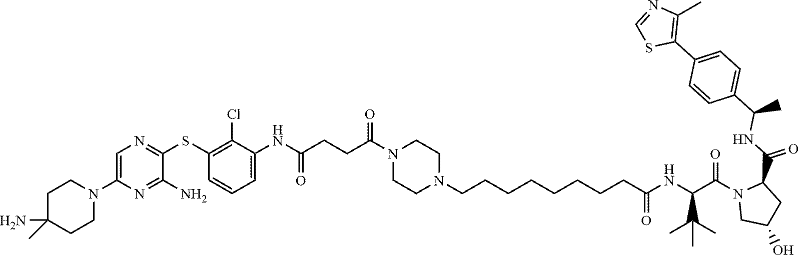 [SMILES: Cc1ncsc1-c1ccc([C@@H](C)NC(=O)[C@H]2C[C@H](O)CN2C(=O)[C@H](NC(=O)CCCCCCCCN2CCN(C(=O)CCC(=O)Nc3cccc(Sc4ncc(N5CCC(C)(N)CC5)nc4N)c3Cl)CC2)C(C)(C)C)cc1] |
| 27 | 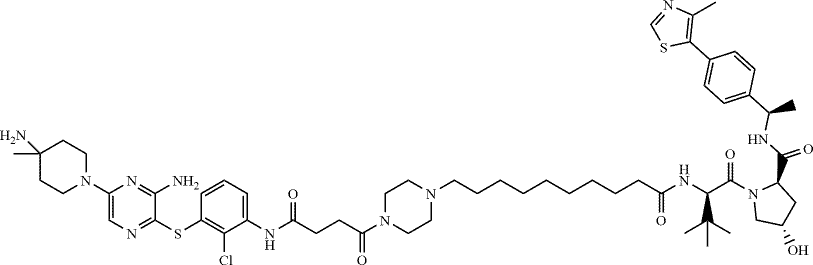 [SMILES: Cc1ncsc1-c1ccc([C@@H](C)NC(=O)[C@H]2C[C@H](O)CN2C(=O)[C@H](NC(=O)CCCCCCCCCN2CCN(C(=O)CCC(=O)Nc3cccc(Sc4ncc(N5CCC(C)(N)CC5)nc4N)c3Cl)CC2)C(C)(C)C)cc1] |
| 28 | 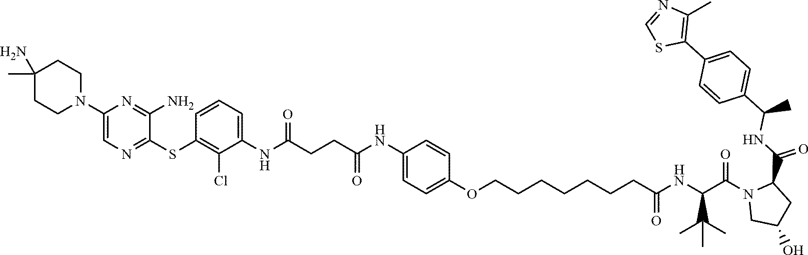 [SMILES: Cc1ncsc1-c1ccc([C@@H](C)NC(=O)[C@H]2C[C@H](O)CN2C(=O)[C@H](NC(=O)CCCCCCCOc2ccc(NC(=O)CCC(=O)Nc3cccc(Sc4ncc(N5CCC(C)(N)CC5)nc4N)c3Cl)cc2)C(C)(C)C)cc1] |
| 29 | 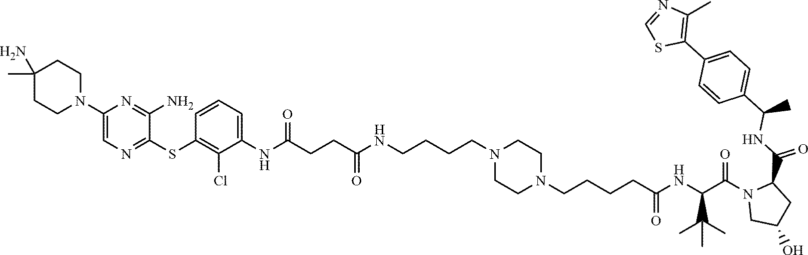 [SMILES: Cc1ncsc1-c1ccc([C@@H](C)NC(=O)[C@H]2C[C@H](O)CN2C(=O)[C@H](NC(=O)CCCCN2CCN(CCCCNC(=O)CCC(=O)Nc3cccc(Sc4ncc(N5CCC(C)(N)CC5)nc4N)c3Cl)CC2)C(C)(C)C)cc1] |
| 30 | 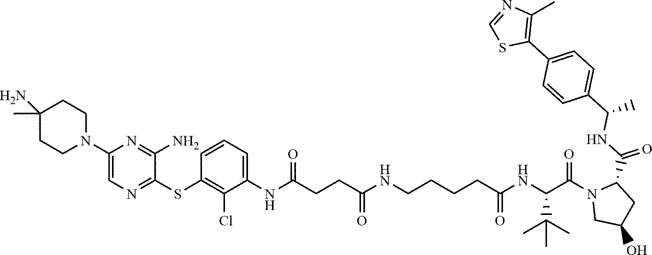 [SMILES: Cc1ncsc1-c1ccc([C@H](C)NC(=O)[C@@H]2C[C@@H](O)CN2C(=O)[C@@H](NC(=O)CCCCNC(=O)CCC(=O)Nc2cccc(Sc3ncc(N4CCC(C)(N)CC4)nc3N)c2Cl)C(C)(C)C)cc1] |
| 31 | 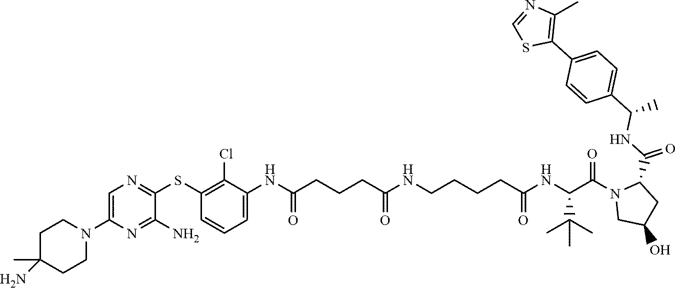 [SMILES: Cc1ncsc1-c1ccc([C@H](C)NC(=O)[C@@H]2C[C@@H](O)CN2C(=O)[C@@H](NC(=O)CCCCNC(=O)CCCC(=O)Nc2cccc(Sc3ncc(N4CCC(C)(N)CC4)nc3N)c2Cl)C(C)(C)C)cc1] |
| 32 | 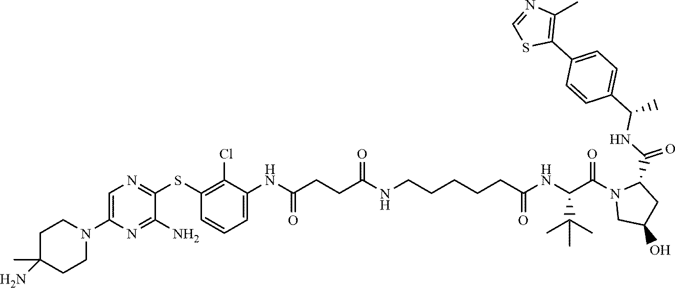 [SMILES: Cc1ncsc1-c1ccc([C@H](C)NC(=O)[C@@H]2C[C@@H](O)CN2C(=O)[C@@H](NC(=O)CCCCCNC(=O)CCC(=O)Nc2cccc(Sc3ncc(N4CCC(C)(N)CC4)nc3N)c2Cl)C(C)(C)C)cc1] |
| 33 | 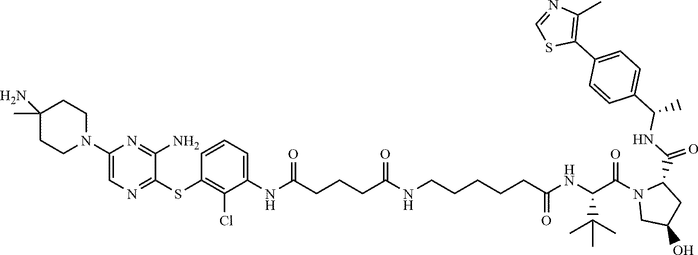 [SMILES: Cc1ncsc1-c1ccc([C@H](C)NC(=O)[C@@H]2C[C@@H](O)CN2C(=O)[C@@H](NC(=O)CCCCCNC(=O)CCCC(=O)Nc2cccc(Sc3ncc(N4CCC(C)(N)CC4)nc3N)c2Cl)C(C)(C)C)cc1] |
| 34 | 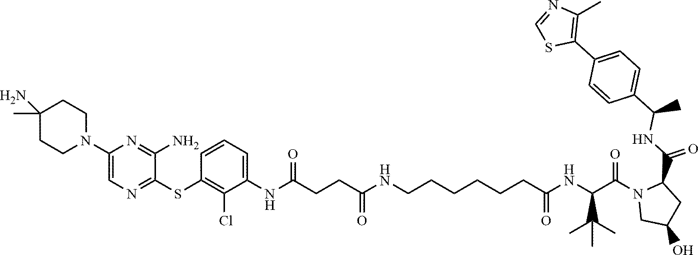 [SMILES: Cc1ncsc1-c1ccc([C@@H](C)NC(=O)[C@H]2C[C@@H](O)CN2C(=O)[C@H](NC(=O)CCCCCCNC(=O)CCC(=O)Nc2cccc(Sc3ncc(N4CCC(C)(N)CC4)nc3N)c2Cl)C(C)(C)C)cc1] |
| 35 | 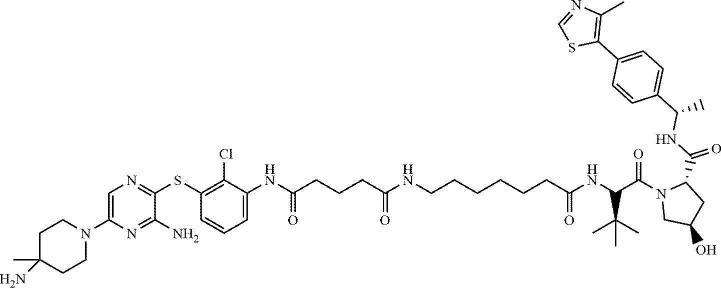 |
| 36 | 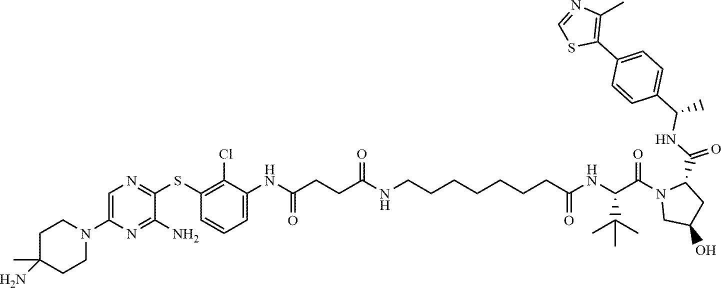 [SMILES: Cc1ncsc1-c1ccc([C@H](C)NC(=O)[C@@H]2C[C@@H](O)CN2C(=O)[C@@H](NC(=O)CCCCCCCNC(=O)CCC(=O)Nc2cccc(Sc3ncc(N4CCC(C)(N)CC4)nc3N)c2Cl)C(C)(C)C)cc1] |
| 37 | 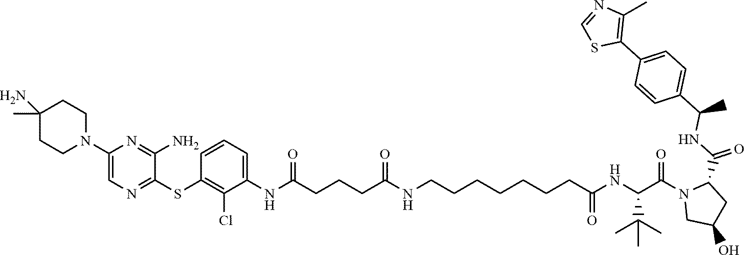 [SMILES: Cc1ncsc1-c1ccc([C@@H](C)NC(=O)[C@@H]2C[C@@H](O)CN2C(=O)[C@@H](NC(=O)CCCCCCCNC(=O)CCCC(=O)Nc2cccc(Sc3ncc(N4CCC(C)(N)CC4)nc3N)c2Cl)C(C)(C)C)cc1] |
| 38 | 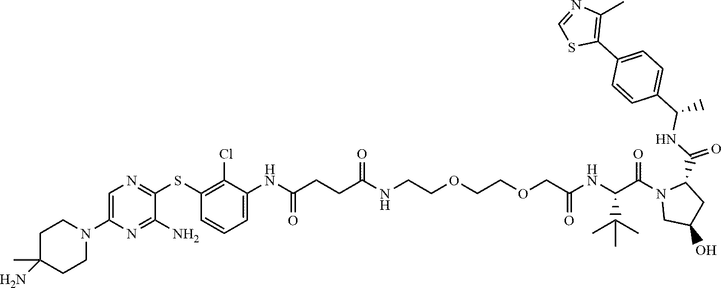 [SMILES: Cc1ncsc1-c1ccc([C@H](C)NC(=O)[C@@H]2C[C@@H](O)CN2C(=O)[C@@H](NC(=O)COCCOCCNC(=O)CCC(=O)Nc2cccc(Sc3ncc(N4CCC(C)(N)CC4)nc3N)c2Cl)C(C)(C)C)cc1] |
| 39 | 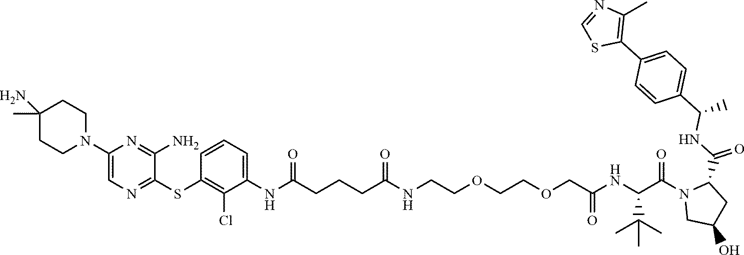 [SMILES: Cc1ncsc1-c1ccc([C@H](C)NC(=O)[C@@H]2C[C@@H](O)CN2C(=O)[C@@H](NC(=O)COCCOCCNC(=O)CCCC(=O)Nc2cccc(Sc3ncc(N4CCC(C)(N)CC4)nc3N)c2Cl)C(C)(C)C)cc1] |
| 40 | 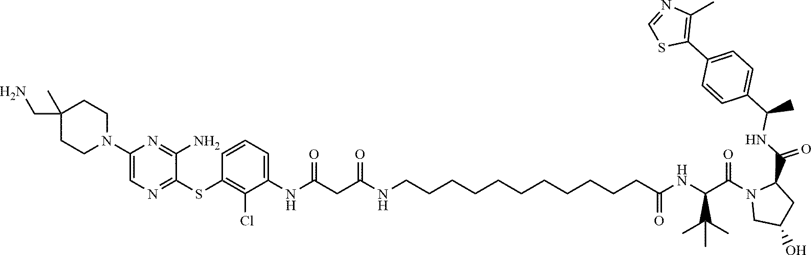 [SMILES: Cc1ncsc1-c1ccc([C@@H](C)NC(=O)[C@H]2C[C@H](O)CN2C(=O)[C@H](NC(=O)CCCCCCCCCCCNC(=O)CC(=O)Nc2cccc(Sc3ncc(N4CCC(C)(CN)CC4)nc3N)c2Cl)C(C)(C)C)cc1] |
| 41 | 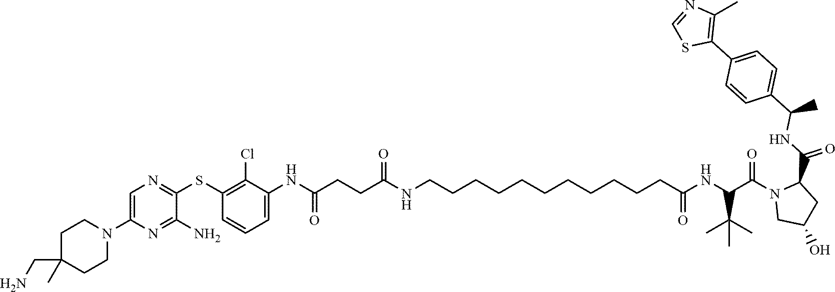 |
| 42 | 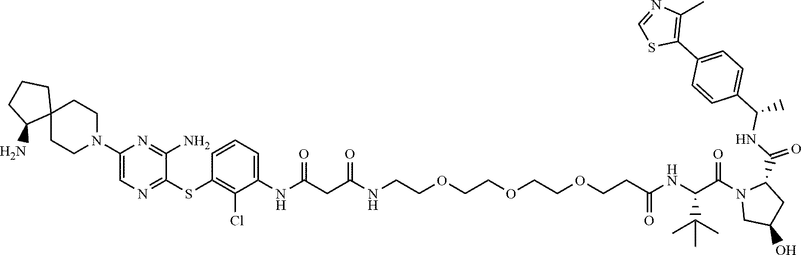 [SMILES: Cc1ncsc1-c1ccc([C@H](C)NC(=O)[C@@H]2C[C@@H](O)CN2C(=O)[C@@H](NC(=O)CCOCCOCCOCCNC(=O)CC(=O)Nc2cccc(Sc3ncc(N4CCC5(CCC[C@@H]5N)CC4)nc3N)c2Cl)C(C)(C)C)cc1] |
| 43 | 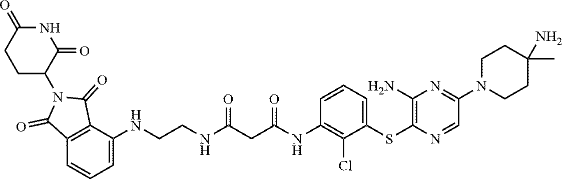 [SMILES: CC1(N)CCN(c2cnc(Sc3cccc(NC(=O)CC(=O)NCCNc4cccc5c4C(=O)N(C4CCC(=O)NC4=O)C5=O)c3Cl)c(N)n2)CC1] |
| 44 | 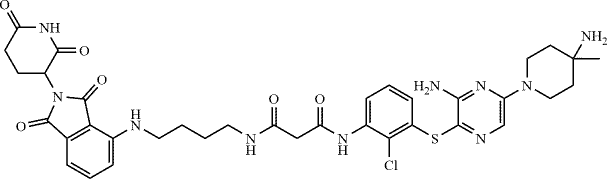 [SMILES: CC1(N)CCN(c2cnc(Sc3cccc(NC(=O)CC(=O)NCCCCNc4cccc5c4C(=O)N(C4CCC(=O)NC4=O)C5=O)c3Cl)c(N)n2)CC1] |
| 45 | 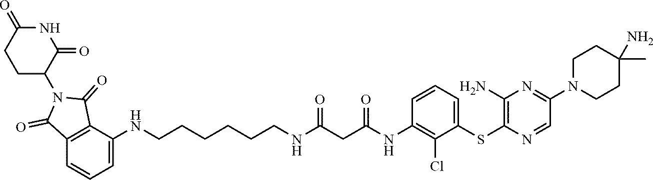 [SMILES: CC1(N)CCN(c2cnc(Sc3cccc(NC(=O)CC(=O)NCCCCCCNc4cccc5c4C(=O)N(C4CCC(=O)NC4=O)C5=O)c3Cl)c(N)n2)CC1] |
| 46 | 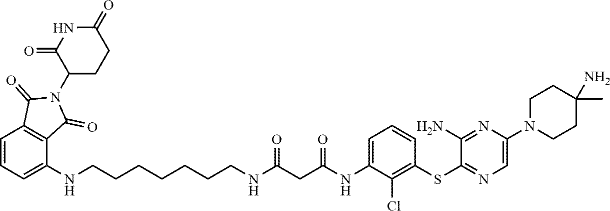 [SMILES: CC1(N)CCN(c2cnc(Sc3cccc(NC(=O)CC(=O)NCCCCCCCNc4cccc5c4C(=O)N(C4CCC(=O)NC4=O)C5=O)c3Cl)c(N)n2)CC1] |
| 47 | 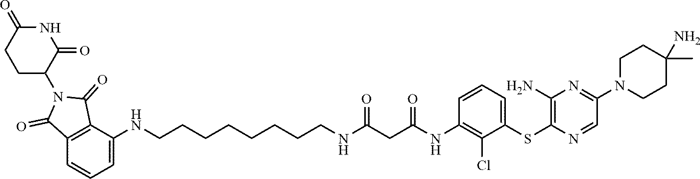 [SMILES: CC1(N)CCN(c2cnc(Sc3cccc(NC(=O)CC(=O)NCCCCCCCCNc4cccc5c4C(=O)N(C4CCC(=O)NC4=O)C5=O)c3Cl)c(N)n2)CC1] |
| 48 | 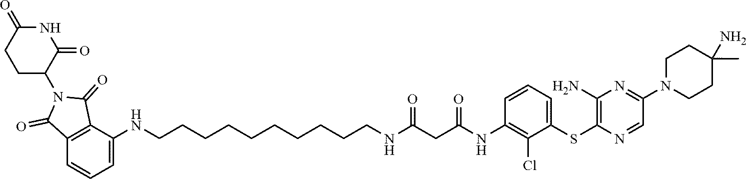 [SMILES: CC1(N)CCN(c2cnc(Sc3cccc(NC(=O)CC(=O)NCCCCCCCCCCNc4cccc5c4C(=O)N(C4CCC(=O)NC4=O)C5=O)c3Cl)c(N)n2)CC1] |
| 49 | 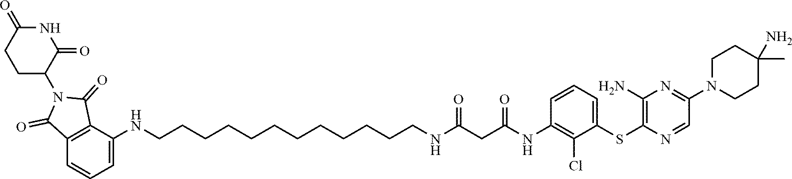 [SMILES: CC1(N)CCN(c2cnc(Sc3cccc(NC(=O)CC(=O)NCCCCCCCCCCCCNc4cccc5c4C(=O)N(C4CCC(=O)NC4=O)C5=O)c3Cl)c(N)n2)CC1] |
| 50 | 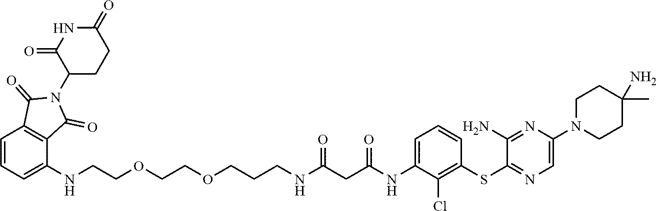 [SMILES: CC1(N)CCN(c2cnc(Sc3cccc(NC(=O)CC(=O)NCCCOCCOCCNc4cccc5c4C(=O)N(C4CCC(=O)NC4=O)C5=O)c3Cl)c(N)n2)CC1] |
| 51 | 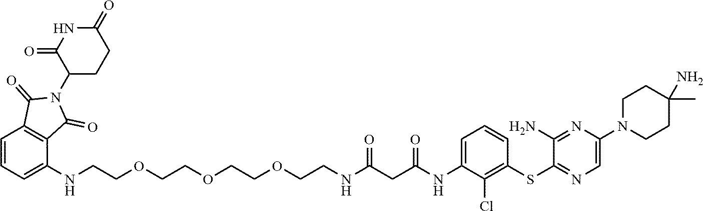 [SMILES: CC1(N)CCN(c2cnc(Sc3cccc(NC(=O)CC(=O)NCCOCCOCCOCCNc4cccc5c4C(=O)N(C4CCC(=O)NC4=O)C5=O)c3Cl)c(N)n2)CC1] |
| 52 | 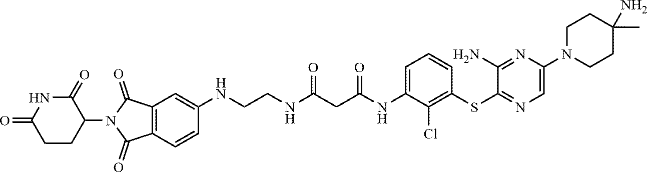 [SMILES: CC1(N)CCN(c2cnc(Sc3cccc(NC(=O)CC(=O)NCCNc4ccc5c(c4)C(=O)N(C4CCC(=O)NC4=O)C5=O)c3Cl)c(N)n2)CC1] |
| 53 | 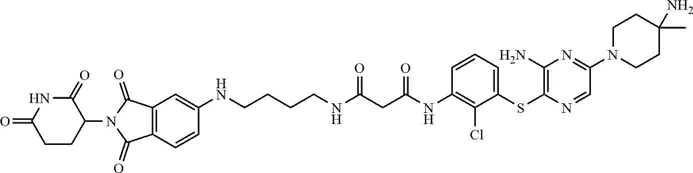 [SMILES: CC1(N)CCN(c2cnc(Sc3cccc(NC(=O)CC(=O)NCCCCNc4ccc5c(c4)C(=O)N(C4CCC(=O)NC4=O)C5=O)c3Cl)c(N)n2)CC1] |
| 54 | 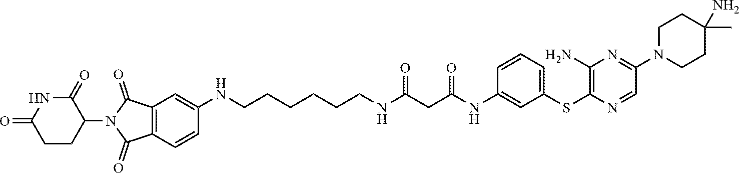 [SMILES: CC1(N)CCN(c2cnc(Sc3cccc(NC(=O)CC(=O)NCCCCCCNc4ccc5c(c4)C(=O)N(C4CCC(=O)NC4=O)C5=O)c3)c(N)n2)CC1] |
| 55 | 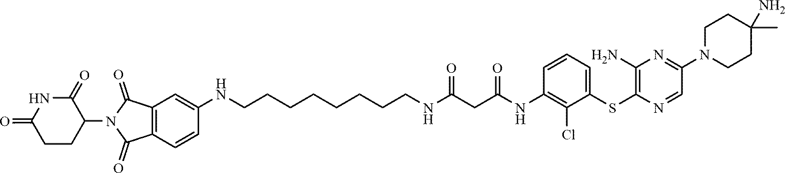 [SMILES: CC1(N)CCN(c2cnc(Sc3cccc(NC(=O)CC(=O)NCCCCCCCCNc4ccc5c(c4)C(=O)N(C4CCC(=O)NC4=O)C5=O)c3Cl)c(N)n2)CC1] |
| 56 | 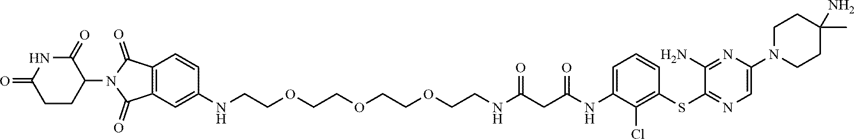 [SMILES: CC1(N)CCN(c2cnc(Sc3cccc(NC(=O)CC(=O)NCCOCCOCCOCCNc4ccc5c(c4)C(=O)N(C4CCC(=O)NC4=O)C5=O)c3Cl)c(N)n2)CC1] |
| 57 | 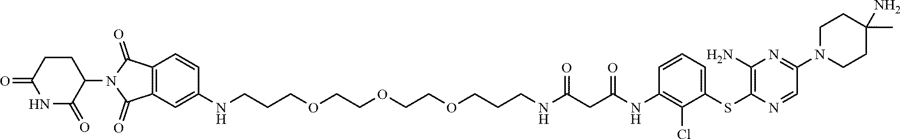 [SMILES: CC1(N)CCN(c2cnc(Sc3cccc(NC(=O)CC(=O)NCCCOCCOCCOCCCNc4ccc5c(c4)C(=O)N(C4CCC(=O)NC4=O)C5=O)c3Cl)c(N)n2)CC1] |
| 58 | 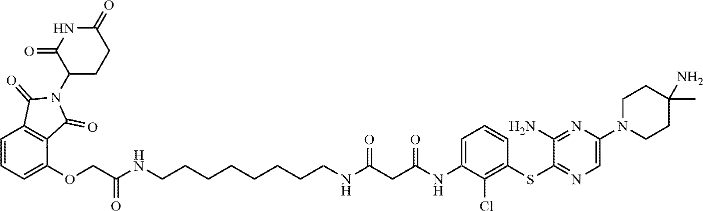 [SMILES: CC1(N)CCN(c2cnc(Sc3cccc(NC(=O)CC(=O)NCCCCCCCCNC(=O)COc4cccc5c4C(=O)N(C4CCC(=O)NC4=O)C5=O)c3Cl)c(N)n2)CC1] |
| 59 | 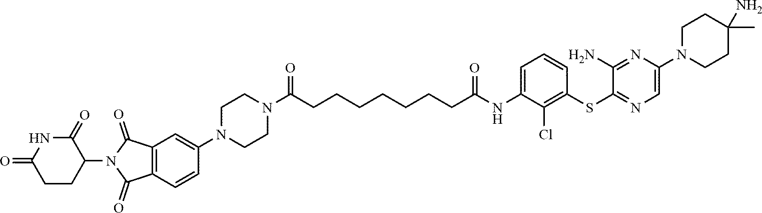 [SMILES: CC1(N)CCN(c2cnc(Sc3cccc(NC(=O)CCCCCCCC(=O)N4CCN(c5ccc6c(c5)C(=O)N(C5CCC(=O)NC5=O)C6=O)CC4)c3Cl)c(N)n2)CC1] |
| 60 | 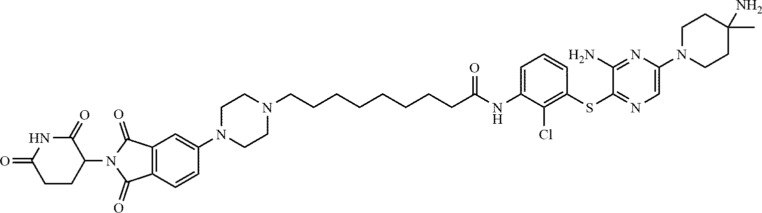 [SMILES: CC1(N)CCN(c2cnc(Sc3cccc(NC(=O)CCCCCCCCN4CCN(c5ccc6c(c5)C(=O)N(C5CCC(=O)NC5=O)C6=O)CC4)c3Cl)c(N)n2)CC1] |
| 61 | 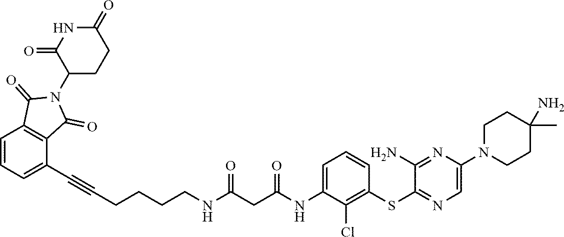 [SMILES: CC1(N)CCN(c2cnc(Sc3cccc(NC(=O)CC(=O)NCCCCC#Cc4cccc5c4C(=O)N(C4CCC(=O)NC4=O)C5=O)c3Cl)c(N)n2)CC1] |

In another embodiment, the disclosure provides a pharmaceutical composition comprising a Compound of the Disclosure and a pharmaceutically acceptable carrier or excipient.

Compounds of the Disclosure may contain an asymmetric carbon atom. In some embodiments, Compounds of the Disclosure are racemic compounds. In other embodiments, Compounds of the Disclosure are enantiomerically enriched, e.g., the enantiomeric excess or “ee” of the compound is about 5% or more as measured by chiral HPLC. In another embodiment, the ee is about 10%. In another embodiment, the ee is about 20%. In another embodiment, the ee is about 30%. In another embodiment, the ee is about 40%. In another embodiment, the ee is about 50%. In another embodiment, the ee is about 60%. In another embodiment, the ee is about 70%. In another embodiment, the ee is about 80%. In another embodiment, the ee is about 85%. In another embodiment, the ee is about 90%. In another embodiment, the ee is about 91%. In another embodiment, the ee is about 92%. In another embodiment, the ee is about 93%. In another embodiment, the ee is about 94%. In another embodiment, the ee is about 95%. In another embodiment, the ee is about 96%. In another embodiment, the ee is about 97%. In another embodiment, the ee is about 98%. In another embodiment, the ee is about 99%.

In another embodiment, the cereblon binding portion of a Compound of the Disclosure, i.e., B¹, is enantiomerically enriched. In another embodiment, the cereblon binding portion of the molecule is racemic. The present disclosure encompasses all possible stereoisomeric, e.g., diastereomeric, forms of Compounds of the Disclosure. For example, all possible stereoisomers of Compounds of the Disclosure are encompassed when A or L portion of Formula I is entantiomerically enriched and the cereblon binding portion of the molecule is racemic. When a Compound of the Disclosure is desired as a single enantiomer, it can be obtained either by resolution of the final product or by stereospecific synthesis from either isomerically pure starting material or use of a chiral auxiliary reagent, for example, see Z. Ma et al., Tetrahedron: Asymmetry, 8(6), pages 883-888 (1997). Resolution of the final product, an intermediate, or a starting material can be achieved by any suitable method known in the art. Additionally, in situations where tautomers of the Compounds of the Disclosure are possible, the present disclosure is intended to include all tautomeric forms of the compounds.

The present disclosure encompasses the preparation and use of salts of Compounds of the Disclosure, including pharmaceutically acceptable salts. As used herein, the “pharmaceutically acceptable salt” refers to non-toxic salt forms of Compounds of the Disclosure. See e.g., Gupta et al., Molecules 23:1719 (2018). Salts of Compounds of the Disclosure can be prepared during the final isolation and purification of the compounds or separately by reacting the compound with an acid having a suitable cation. The pharmaceutically acceptable salts of Compounds of the Disclosure can be acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, and citric. Nonlimiting examples of salts of compounds of the disclosure include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphsphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulfonate, and p-toluenesulfonate salts. In addition, available amino groups present in the compounds of the disclosure can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. In light of the foregoing, any reference Compounds of the Disclosure appearing herein is intended to include the actual compound as well as pharmaceutically acceptable salts, hydrates, or solvates thereof.

The present disclosure also encompasses the preparation and use of solvates of Compounds of the Disclosure. Solvates typically do not significantly alter the physiological activity or toxicity of the compounds, and as such may function as pharmacological equivalents. The term “solvate” as used herein is a combination, physical association and/or solvation of a compound of the present disclosure with a solvent molecule such as, e.g. a disolvate, monosolvate or hemisolvate, where the ratio of solvent molecule to compound of the present disclosure is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. Compounds of the Disclosure can be present as solvated forms with a pharmaceutically acceptable solvent, such as water, methanol, and ethanol, and it is intended that the disclosure includes both solvated and unsolvated forms of Compounds of the Disclosure. One type of solvate is a hydrate. A “hydrate” relates to a particular subgroup of solvates where the solvent molecule is water. Solvates typically can function as pharmacological equivalents. Preparation of solvates is known in the art. See, for example, M. Caira et al, J. Pharmaceut. Sci., 93(3):601-611 (2004), which describes the preparation of solvates of fluconazole with ethyl acetate and with water. Similar preparation of solvates, hemisolvates, hydrates, and the like are described by E.C. van Tonder et al., AAPS Pharm. Sci. Tech., 5(1):Article 12 (2004), and A.L. Bingham et al., Chem. Commun. 603-604 (2001). A typical, non-limiting, process of preparing a solvate would involve dissolving a Compound of the Disclosure in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvent in a crystal of the solvate.

### II. Therapeutic Methods of the Disclosure

Compounds of the Disclosure degrade SHP2 protein and are thus useful in the treatment of a variety of diseases and conditions. In particular, Compounds of the Disclosure are useful in methods of treating a disease or condition wherein degradation SHP2 proteins provides a benefit, for example, cancers and proliferative diseases. The therapeutic methods of the disclosure comprise administering a therapeutically effective amount of a Compound of the Disclosure to a subject, e.g., a cancer patient, in need thereof. The present methods also encompass administering a second therapeutic agent to the subject in combination with the Compound of the Disclosure. The second therapeutic agent is selected from drugs known as useful in treating the disease or condition afflicting the individual in need thereof, e.g., a chemotherapeutic agent and/or radiation known as useful in treating a particular cancer.

The present disclosure provides Compounds of the Disclosure as SHP2 protein degraders for the treatment of a variety of diseases and conditions wherein degradation of SHP2 proteins has a beneficial effect. Compounds of the Disclosure typically have DC₅₀ (the drug concentration that results in 50% SHP2 protein degradation) values of less than 100 µM, e.g., less than 50 µM, less than 25 µM, and less than 5 µM, less than about 1 µM, less than about 0.5 µM, or less than about 0.1 µM. In some embodiments, Compounds of the Disclosure typically have DC₅₀ values of less than about 0.01 µM. In some embodiments, Compounds of the Disclosure typically have DC₅₀ values of less than about 0.001 µM. In one embodiment, the present disclosure relates to a method of treating an individual suffering from a disease or condition wherein degradation of SHP2 proteins provides a benefit comprising administering a therapeutically effective amount of a Compound of the Disclosure to an individual in need thereof.

Since Compounds of the Disclosure are degraders of SHP2 protein, a number of diseases and conditions mediated by SHP2 can be treated by employing these compounds. The present disclosure is thus directed generally to a method for treating a condition or disorder responsive to degradation of SHP2 in an animal, e.g., a human, suffering from, or at risk of suffering from, the condition or disorder, the method comprising administering to the animal an effective amount of one or more Compounds of the Disclosure.

The present disclosure is further directed to a method of degrading SHP2 protein in a subject in need thereof, said method comprising administering to the subject an effective amount of at least one Compound of the Disclosure.

In another aspect, the present disclosure provides a method of treating cancer in a subject comprising administering a therapeutically effective amount of a Compound of the Disclosure. While not being limited to a specific mechanism, in some embodiments, Compounds of the Disclosure treat cancer by degrading SHP2. Examples of treatable cancers include, but are not limited to, any one or more of the cancers of Table 2.

TABLE 2

| | | | |
|---|---|---|---|
| adrenal cancer | acinic cell carcinoma | acoustic neuroma | acral lentigious melanoma |
| acrospiroma | acute eosinophilic leukemia | acute erythroid leukemia | acute lymphoblastic leukemia |
| acute megakaryoblastic leukemia | acute monocytic leukemia | acute promyelocytic leukemia | adenocarcinoma |
| adenoid cystic carcinoma | adenoma | adenomatoid odontogenic tumor | adenosquamous carcinoma |
| adipose tissue neoplasm | adrenocortical carcinoma | adult T-cell leukemia/lymphoma | aggressive NK-cell leukemia |
| AIDS-related lymphoma | alveolar rhabdomyosarcoma | alveolar soft part sarcoma | ameloblastic fibroma |
| anaplastic large cell lymphoma | anaplastic thyroid cancer | angioimmunoblastic T-cell lymphoma | angiomyolipoma |
| angiosarcoma | astrocytoma | atypical teratoid rhabdoid tumor | B-cell chronic lymphocytic leukemia |
| B-cell prolymphocytic leukemia | B-cell lymphoma | basal cell carcinoma | biliary tract cancer |
| bladder cancer | blastoma | bone cancer | Brenner tumor |
| Brown tumor | Burkitt’s lymphoma | breast cancer | brain cancer |
| carcinoma | carcinoma in situ | carcinosarcoma | cartilage tumor |
| cementoma | myeloid sarcoma | chondroma | chordoma |
| choriocarcinoma | choroid plexus papilloma | clear-cell sarcoma of the kidney | craniopharyngioma |
| cutaneous T-cell lymphoma | cervical cancer | colorectal cancer | Degos disease |
| desmoplastic small round cell tumor | diffuse large B-cell lymphoma | dysembryoplastic neuroepithelial tumor | dysgerminoma |
| embryonal carcinoma | endocrine gland neoplasm | endodermal sinus tumor | enteropathy-associated T-cell lymphoma |
| esophageal cancer | fetus in fetu | fibroma | fibrosarcoma |
| follicular lymphoma | follicular thyroid cancer | ganglioneuroma | gastrointestinal cancer |
| germ cell tumor | gestational choriocarcinoma | giant cell fibroblastoma | giant cell tumor of the bone |
| glial tumor | glioblastoma multiforme | glioma | gliomatosis cerebri |
| glucagonoma | gonadoblastoma | granulosa cell tumor | gynandroblastoma |
| gallbladder cancer | gastric cancer | hairy cell leukemia | hemangioblastoma |
| head and neck cancer | hemangiopericytoma | hematological cancer | hepatoblastoma |
| hepatosplenic T-cell lymphoma | Hodgkin’s lymphoma | non-Hodgkin’s lymphoma | invasive lobular carcinoma |
| intestinal cancer | kidney cancer | laryngeal cancer | lentigo maligna |
| lethal midline carcinoma | leukemia | leydig cell tumor | liposarcoma |
| lung cancer | lymphangioma | lymphangiosarcoma | lymphoepithelioma |
| lymphoma | acute lymphocytic leukemia | acute myelogeous leukemia | chronic lymphocytic leukemia |
| liver cancer | small cell lung cancer | non-small cell lung cancer | MALT lymphoma |
| malignant fibrous histiocytoma | malignant peripheral nerve sheath tumor | malignant triton tumor | mantle cell lymphoma |
| marginal zone B-cell lymphoma | mast cell leukemia | mediastinal germ cell tumor | medullary carcinoma of the breast |
| medullary thyroid cancer | medulloblastoma | melanoma | meningioma |
| merkel cell cancer | mesothelioma | metastatic urothelial carcinoma | mixed Mullerian tumor |
| mucinous tumor | multiple myeloma | muscle tissue neoplasm | mycosis fungoides |
| myxoid liposarcoma | myxoma | myxosarcoma | nasopharyngeal carcinoma |
| neurinoma | neuroblastoma | neurofibroma | neuroma |
| nodular melanoma | ocular cancer | oligoastrocytoma | oligodendroglioma |
| oncocytoma | optic nerve sheath meningioma | optic nerve tumor | oral cancer |
| osteosarcoma | ovarian cancer | Pancoast tumor | papillary thyroid cancer |
| paraganglioma | pinealoblastoma | pineocytoma | pituicytoma |
| pituitary adenoma | pituitary tumor | plasmacytoma | polyembryoma |
| precursor T-lymphoblastic lymphoma | primary central nervous system lymphoma | primary effusion lymphoma | preimary peritoneal cancer |
| prostate cancer | pancreatic cancer | pharyngeal cancer | pseudomyxoma periotonei |
| renal cell carcinoma | renal medullary carcinoma | retinoblastoma | rhabdomyoma |
| rhabdomyosarcoma | Richter’s transformation | rectal cancer | sarcoma |
| Schwannomatosis | seminoma | Sertoli cell tumor | sex cord-gonadal stromal tumor |
| signet ring cell carcinoma | skin cancer | small blue round cell tumors | small cell carcinoma |
| soft tissue sarcoma | somatostatinoma | soot wart | spinal tumor |
| splenic marginal zone lymphoma | squamous cell carcinoma | synovial sarcoma | Sezary’s disease |
| small intestine cancer | squamous carcinoma | stomach cancer | T-cell lymphoma |
| testicular cancer | thecoma | thyroid cancer | transitional cell carcinoma |
| throat cancer | urachal cancer | urogenital cancer | urothelial carcinoma |
| uveal melanoma | uterine cancer | verrucous carcinoma | visual pathway glioma |
| vulvar cancer | vaginal cancer | Waldenstrom’s macroglobulinemia | Warthin’s tumor |
| Wilms’ tumor | | | |

In another embodiment, the cancer is a solid tumor. In another embodiment, the cancer a hematological cancer. Exemplary hematological cancers include, but are not limited to, the cancers listed in Table 3. In another embodiment, the hematological cancer is acute lymphocytic leukemia, chronic lymphocytic leukemia (including B-cell chronic lymphocytic leukemia), or acute myeloid leukemia.

TABLE 3

| | |
|---|---|
| acute lymphocytic leukemia (ALL) | acute eosinophilic leukemia |
| acute myeloid leukemia (AML) | acute erythroid leukemia |
| chronic lymphocytic leukemia (CLL) | acute lymphoblastic leukemia |
| small lymphocytic lymphoma (SLL) | acute megakaryoblastic leukemia |
| multiple myeloma (MM) | acute monocytic leukemia |
| Hodgkins lymphoma (HL) | acute promyelocytic leukemia |
| non-Hodgkin’s lymphoma (NHL) | acute myelogeous leukemia |
| mantle cell lymphoma (MCL) | B-cell prolymphocytic leukemia |
| marginal zone B-cell lymphoma | B-cell lymphoma |
| splenic marginal zone lymphoma | MALT lymphoma |
| follicular lymphoma (FL) | precursor T-lymphoblastic lymphoma |
| Waldenstrom’s macroglobulinemia (WM) | T-cell lymphoma |
| diffuse large B-cell lymphoma (DLBCL) | mast cell leukemia |
| marginal zone lymphoma (MZL) | adult T cell leukemia/lymphoma |
| hairy cell leukemia (HCL) | aggressive NK-cell leukemia |
| Burkitt’s lymphoma (BL) | angioimmunoblastic T-cell lymphoma |
| Richter’s transformation | |

In another embodiment, the cancer is a leukemia, for example a leukemia selected from acute monocytic leukemia, acute myelogenous leukemia, chronic myelogenous leukemia, chronic lymphocytic leukemia and mixed lineage leukemia (MLL). In another embodiment the cancer is NUT-midline carcinoma. In another embodiment the cancer is multiple myeloma. In another embodiment the cancer is a lung cancer such as small cell lung cancer (SCLC). In another embodiment the cancer is a neuroblastoma. In another embodiment the cancer is Burkitt’s lymphoma. In another embodiment the cancer is cervical cancer. In another embodiment the cancer is esophageal cancer. In another embodiment the cancer is ovarian cancer. In another embodiment the cancer is colorectal cancer. In another embodiment, the cancer is prostate cancer. In another embodiment, the cancer is breast cancer.

In another embodiment, the cancer is selected from the group consisting of acute monocytic leukemia, acute myelogenous leukemia, chronic myelogenous leukemia, chronic lymphocytic leukemia mixed lineage leukemia, NUT-midline carcinoma, multiple myeloma, small cell lung cancer, non-small cell lung cancer, neuroblastoma, Burkitt’s lymphoma, cervical cancer, esophageal cancer, ovarian cancer, colorectal cancer, prostate cancer, breast cancer, bladder cancer, ovary cancer, glioma, sarcoma, esophageal squamous cell carcinoma, and papillary thyroid carcinoma.

In another embodiment, Compounds of the Disclosure are administered to a subject in need thereof to treat breast cancer or prostate cancer. In another embodiment, the cancer is breast cancer. In another embodiment, the cancer is prostate cancer. In another embodiment, the cancer is metastatic castration-resistant prostate cancer.

The methods of the present disclosure can be accomplished by administering a Compound of the Disclosure as the neat compound or as a pharmaceutical composition. Administration of a pharmaceutical composition, or neat Compound of the Disclosure, can be performed during or after the onset of the disease or condition of interest. Typically, the pharmaceutical compositions are sterile, and contain no toxic, carcinogenic, or mutagenic compounds that would cause an adverse reaction when administered.

In one embodiment, a Compound of the Disclosure is administered as a single agent to treat a disease or condition wherein degradation of SHP2 protein provides a benefit. In another embodiment, a Compound of the Disclosure is administered in conjunction with a second therapeutic agent useful in the treatment of a disease or condition wherein degradation of SHP2 protein provides a benefit. The second therapeutic agent is different from the Compound of the Disclosure. A Compound of the Disclosure and the second therapeutic agent can be administered simultaneously or sequentially to achieve the desired effect. In addition, the Compound of the Disclosure and second therapeutic agent can be administered as a single pharmaceutical composition or two separate pharmaceutical compositions.

The second therapeutic agent is administered in an amount to provide its desired therapeutic effect. The effective dosage range for each second therapeutic agent is known in the art, and the second therapeutic agent is administered to an individual in need thereof within such established ranges.

A Compound of the Disclosure and the second therapeutic agent can be administered together as a single-unit dose or separately as multi-unit doses, wherein the Compound of the Disclosure is administered before the second therapeutic agent or vice versa. One or more doses of the Compound of the Disclosure and/or one or more doses of the second therapeutic agent can be administered. The Compound of the Disclosure therefore can be used in conjunction with one or more second therapeutic agents, for example, but not limited to, anticancer agents.

In methods of the present disclosure, a therapeutically effective amount of a Compound of the Disclosure, typically formulated in accordance with pharmaceutical practice, is administered to a subject, e.g., a human cancer patient, in need thereof. Whether such a treatment is indicated depends on the individual case and is subject to medical assessment (diagnosis) that takes into consideration signs, symptoms, and/or malfunctions that are present, the risks of developing particular signs, symptoms and/or malfunctions, and other factors.

A Compound of the Disclosure can be administered by any suitable route, for example by oral, buccal, inhalation, sublingual, rectal, vaginal, intracisternal or intrathecal through lumbar puncture, transurethral, nasal, percutaneous, i.e., transdermal, or parenteral (including intravenous, intramuscular, subcutaneous, intracoronary, intradermal, intramammary, intraperitoneal, intraarticular, intrathecal, retrobulbar, intrapulmonary injection and/or surgical implantation at a particular site) administration. Parenteral administration can be accomplished using a needle and syringe or using a high pressure technique.

Pharmaceutical compositions include those wherein a Compound of the Disclosure is administered in an effective amount to achieve its intended purpose. The exact formulation, route of administration, and dosage is determined by an individual physician in view of the diagnosed condition or disease. Dosage amount and interval can be adjusted individually to provide levels of a Compound of the Disclosure that is sufficient to maintain therapeutic effects.

Toxicity and therapeutic efficacy of the Compounds of the Disclosure can be determined by standard pharmaceutical procedures in cell cultures or experimental animals, e.g., for determining the maximum tolerated dose (MTD) of a compound, which defines as the highest dose that causes no toxicity in animals. The dose ratio between the maximum tolerated dose and therapeutic effects (e.g. inhibiting of tumor growth) is the therapeutic index. The dosage can vary within this range depending upon the dosage form employed, and the route of administration utilized. Determination of a therapeutically effective amount is well within the capability of those skilled in the art, especially in light of the detailed disclosure provided herein.

A therapeutically effective amount of a Compound of the Disclosure required for use in therapy varies with the nature of the condition being treated, the length of time that activity is desired, and the age and the condition of the patient, and ultimately is determined by the attendant physician. Dosage amounts and intervals can be adjusted individually to provide plasma levels of the SHP2 protein degrader that are sufficient to maintain the desired therapeutic effects. The desired dose conveniently can be administered in a single dose, or as multiple doses administered at appropriate intervals, for example as one, two, three, four or more subdoses per day. Multiple doses often are desired, or required. For example, a Compound of the Disclosure can be administered at a frequency of: four doses delivered as one dose per day at four-day intervals (q4d x 4); four doses delivered as one dose per day at three-day intervals (q3d x 4); one dose delivered per day at five-day intervals (qd x 5); one dose per week for three weeks (qwk3); five daily doses, with two days rest, and another five daily doses (5/2/5); or, any dose regimen determined to be appropriate for the circumstance.

A Compound of the Disclosure used in a method of the present disclosure can be administered in an amount of about 0.005 to about 500 milligrams per dose, about 0.05 to about 250 milligrams per dose, or about 0.5 to about 100 milligrams per dose. For example, a Compound of the Disclosure can be administered, per dose, in an amount of about 0.005, 0.05, 0.5, 5, 10, 20, 30, 40, 50, 100, 150, 200, 250, 300, 350, 400, 450, or 500 milligrams, including all doses between 0.005 and 500 milligrams.

The dosage of a composition containing a Compound of the Disclosure, or a composition containing the same, can be from about 1 ng/kg to about 200 mg/kg, about 1 µg/kg to about 100 mg/kg, or about 1 mg/kg to about 50 mg/kg. The dosage of a composition can be at any dosage including, but not limited to, about 1 µg/kg. The dosage of a composition may be at any dosage including, but not limited to, about 1 µg/kg, about 10 µg/kg, about 25 µg/kg, about 50 µg/kg, about 75 µg/kg, about 100 µg/kg, about 125 µg/kg, about 150 µg/kg, about 175 µg/kg, about 200 µg/kg, about 225 µg/kg, about 250 µg/kg, about 275 µg/kg, about 300 µg/kg, about 325 µg/kg, about 350 µg/kg, about 375 µg/kg, about 400 µg/kg, about 425 µg/kg, about 450 µg/kg, about 475 µg/kg, about 500 µg/kg, about 525 µg/kg, about 550 µg/kg, about 575 µg/kg, about 600 µg/kg, about 625 µg/kg, about 650 µg/kg, about 675 µg/kg, about 700 µg/kg, about 725 µg/kg, about 750 µg/kg, about 775 µg/kg, about 800 µg/kg, about 825 µg/kg, about 850 µg/kg, about 875 µg/kg, about 900 µg/kg, about 925 µg/kg, about 950 µg/kg, about 975 µg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 125 mg/kg, about 150 mg/kg, about 175 mg/kg, about 200 mg/kg, or more. The above dosages are exemplary of the average case, but there can be individual instances in which higher or lower dosages are merited, and such are within the scope of this disclosure. In practice, the physician determines the actual dosing regimen that is most suitable for an individual patient, which can vary with the age, weight, and response of the particular patient.

In another embodiment, chemotherapeutic agents or other anti-proliferative agents can be combined with Compound of the Disclosure to treat proliferative diseases and cancer. Examples of therapies and anticancer agents that can be used in combination with Compounds of the Disclosure include surgery, radiotherapy (e.g., gamma-radiation, neutron beam radiotherapy, electron beam radiotherapy, proton therapy, brachytherapy, and systemic radioactive isotopes), endocrine therapy, a biologic response modifier (e.g., an interferon, an interleukin, tumor necrosis factor (TNF), hyperthermia and cryotherapy, an agent to attenuate any adverse effect (e.g., an antiemetic), and any other approved chemotherapeutic drug.

Compounds of the Disclosure typically are administered in admixture with a pharmaceutical carrier selected with regard to the intended route of administration and standard pharmaceutical practice. Pharmaceutical compositions for use in accordance with the present disclosure are formulated in a conventional manner using one or more physiologically acceptable carriers comprising excipients and/or auxiliaries that facilitate processing of Compound of the Disclosure.

These pharmaceutical compositions can be manufactured, for example, by conventional mixing, dissolving, granulating, dragee-making, emulsifying, encapsulating, entrapping, or lyophilizing processes. Proper formulation is dependent upon the route of administration chosen. When a therapeutically effective amount of the Compound of the Disclosure is administered orally, the composition typically is in the form of a tablet, capsule, powder, solution, or elixir. When administered in tablet form, the composition additionally can contain a solid carrier, such as a gelatin or an adjuvant. The tablet, capsule, and powder contain about 0.01% to about 95%, and preferably from about 1% to about 50%, of a Compound of the Disclosure. When administered in liquid form, a liquid carrier, such as water, petroleum, or oils of animal or plant origin, can be added. The liquid form of the composition can further contain physiological saline solution, dextrose or other saccharide solutions, or glycols. When administered in liquid form, the composition contains about 0.1% to about 90%, and preferably about 1% to about 50%, by weight, of a Compound of the Disclosure.

When a therapeutically effective amount of a Compound of the Disclosure is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains, an isotonic vehicle.

Compounds of the Disclosure can be readily combined with pharmaceutically acceptable carriers well-known in the art. Standard pharmaceutical carriers are described in Remington’s Pharmaceutical Sciences, Mack Publishing Co., Easton, PA, 19th ed. 1995. Such carriers enable the active agents to be formulated as tablets, pills, dragees, capsules, liquids, gels, syrups, slurries, suspensions and the like, for oral ingestion by a patient to be treated. Pharmaceutical preparations for oral use can be obtained by adding the Compound of the Disclosure to a solid excipient, optionally grinding the resulting mixture, and processing the mixture of granules, after adding suitable auxiliaries, if desired, to obtain tablets or dragee cores.

Suitable excipients include fillers such as saccharides (for example, lactose, sucrose, mannitol or sorbitol), cellulose preparations, calcium phosphates (for example, tricalcium phosphate or calcium hydrogen phosphate), as well as binders such as starch paste (using, for example, maize starch, wheat starch, rice starch, or potato starch), gelatin, tragacanth, methyl cellulose, hydroxypropylmethylcellulose, sodium carboxymethylcellulose, and/or polyvinyl pyrrolidone. If desired, one or more disintegrating agents can be added, such as the above-mentioned starches and also carboxymethyl-starch, cross-linked polyvinyl pyrrolidone, agar, or alginic acid or a salt thereof, such as sodium alginate. Buffers and pH modifiers can also be added to stabilize the pharmaceutical composition.

Auxiliaries are typically flow-regulating agents and lubricants such as, for example, silica, talc, stearic acid or salts thereof (e.g., magnesium stearate or calcium stearate), and polyethylene glycol. Dragee cores are provided with suitable coatings that are resistant to gastric juices. For this purpose, concentrated saccharide solutions can be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate can be used. Dye stuffs or pigments can be added to the tablets or dragee coatings, for example, for identification or in order to characterize combinations of active compound doses.

Compound of the Disclosure can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical compositions for parenteral administration include aqueous solutions of the active agent in water-soluble form. Additionally, suspensions of a Compound of the Disclosure can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

Compounds of the Disclosure also can be formulated in rectal compositions, such as suppositories or retention enemas, e.g., containing conventional suppository bases. In addition to the formulations described previously, the Compound of the Disclosure also can be formulated as a depot preparation. Such long-acting formulations can be administered by implantation (for example, subcutaneously or intramuscularly) or by intramuscular injection. Thus, for example, the Compound of the Disclosure can be formulated with suitable polymeric or hydrophobic materials (for example, as an emulsion in an acceptable oil) or ion exchange resins.

In particular, the Compounds of the Disclosure can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. Compound of the Disclosure also can be injected parenterally, for example, intravenously, intramuscularly, subcutaneously, or intracoronarily. For parenteral administration, the Compound of the Disclosure are typically used in the form of a sterile aqueous solution which can contain other substances, for example, salts or monosaccharides, such as mannitol or glucose, to make the solution isotonic with blood.

The disclosure provides the following particular embodiments in connection with treating a disease in a subject with a Compound of the Disclosure.

Embodiment I. A method of treating a subject, the method comprising administering to the subject a therapeutically effective amount of a Compound of the Disclosure, wherein the subject has cancer, a chronic autoimmune disorder, an inflammatory condition, a proliferative disorder, sepsis, or a viral infection.

Embodiment II. The method Embodiment I, wherein the subject has cancer, e.g., any one of more of the cancers of Table 2 or Table 3.

Embodiment III. The method of Embodiment II, wherein the cancer is prostate cancer or breast cancer.

Embodiment IV. The method of Embodiment II, wherein the cancer is breast cancer.

Embodiment V. The method of Embodiment II, wherein the cancer is prostate cancer, e.g., metastatic castration-resistant prostate cancer.

Embodiment VI. The method of any one of Embodiments I-V further comprising administering a therapeutically effective amount of a second therapeutic agent useful in the treatment of the disease or condition, e.g., an immune checkpoint inhibitor or other anticancer agent.

Embodiment VII. A pharmaceutical composition comprising a Compound of the Disclosure and a pharmaceutically acceptable excipient for use in treating cancer, a chronic autoimmune disorder, an inflammatory condition, a proliferative disorder, sepsis, or a viral infection.

Embodiment VIII. The pharmaceutical composition of Embodiment VII for use in treating cancer.

Embodiment IX. The pharmaceutical composition of Embodiment VIII, wherein the cancer is prostate cancer or breast cancer.

Embodiment X. The pharmaceutical composition of Embodiment VIII, wherein the cancer is breast cancer.

Embodiment XI. The pharmaceutical composition of Embodiment VIII, wherein the cancer is prostate cancer, e.g., metastatic castration-resistant prostate cancer.

Embodiment XII. A Compound of the Disclosure for use in treatment of cancer, a chronic autoimmune disorder, an inflammatory condition, a proliferative disorder, sepsis, or a viral infection.

Embodiment XIII. The compound of Embodiment XIII for use in treating cancer.

Embodiment XIV. The compound of Embodiment XIII, wherein the cancer is breast cancer.

Embodiment XV. The compound of Embodiment XIII, wherein the cancer is prostate cancer, e.g., metastatic castration-resistant prostate cancer.

Embodiment XVI. Use of a Compound of the Disclosure for the manufacture of a medicament for treatment of cancer, a chronic autoimmune disorder, an inflammatory condition, a proliferative disorder, sepsis, or a viral infection.

Embodiment XVII. The use of Embodiment XVI for the treatment of cancer.

Embodiment XVIII. The use of Embodiment XVII, wherein the cancer is prostate cancer or breast cancer.

Embodiment XIV. The use of Embodiment XVII, wherein the cancer is breast cancer.

Embodiment XX. The use of Embodiment XVII, wherein the cancer is prostate cancer, e.g., metastatic castration-resistant prostate cancer.

Embodiment XXI. A method of reducing SHP2 protein within a cell of a subject in need thereof, the method comprising administering to the subject a Compound of the Disclosure. In one embodiment, the SHP2 protein is reduced by about 50% or less, e.g., 1%, about 2%, about 3%, about 4%, about 5%, about 10%, about 15%, about 20%, about 25%, about 30%, about 35%, about 40%, or about 45%. In one embodiment, the SHP2 protein is reduced by about 51% or more, e.g., about 55%, about 60%, about 65%, about 70%, about 75%, about 80%, about 85%, about 90%, or about 95%.

### III. Kits of the Disclosure

In another embodiment, the present disclosure provides kits which comprise a Compound of the Disclosure (or a composition comprising a Compound of the Disclosure) packaged in a manner that facilitates its use to practice methods of the present disclosure. In one embodiment, the kit includes a Compound of the Disclosure (or a composition comprising a Compound of the Disclosure) packaged in a container, such as a sealed bottle or vessel, with a label affixed to the container or included in the kit that describes use of the compound or composition to practice the method of the disclosure. In one embodiment, the compound or composition is packaged in a unit dosage form. The kit further can include a device suitable for administering the composition according to the intended route of administration.

### IV. Definitions

The term “a disease or condition wherein degradation of Src homology 2 domain-containing phosphatase (SHP2) protein provides a benefit” and the like pertains to a disease or condition in which SHP2 is important or necessary, e.g., for the onset, progress, expression of that disease or condition, or a disease or a condition which is known to be treated by an SHP2 degrader. Examples of such conditions include, but are not limited to, a cancer. One of ordinary skill in the art is readily able to determine whether a compound treats a disease or condition mediated by an SHP2 degrader for any particular cell type, for example, by assays which conveniently can be used to assess the activity of particular compounds.

The term “SHP2 degrader,” and the like refer to a heterobifunctional small molecule that degrades SHP2 protein. SHP2 degraders contain a first ligand which binds to SHP2 protein, a second ligand for an E3 ligase system, and a chemical linker that tethers the first and second ligands. Representative Compounds of the Disclosure that degrade SHP2 protein are disclosed in Table 1.

The term “second therapeutic agent” refers to a therapeutic agent different from a Compound of the Disclosure and that is known to treat the disease or condition of interest. For example when a cancer is the disease or condition of interest, the second therapeutic agent can be a known chemotherapeutic drug, like taxol, or radiation, for example.

The term “disease” or “condition” denotes disturbances and/or anomalies that as a rule are regarded as being pathological conditions or functions, and that can manifest themselves in the form of particular signs, symptoms, and/or malfunctions. Compounds of the Disclosure are degraders of SHP2 and can be used in treating or preventing diseases and conditions wherein degradation of SHP2 provides a benefit.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. The term “treat” and synonyms contemplate administering a therapeutically effective amount of a Compound of the Disclosure to a subject in need of such treatment. The treatment can be orientated symptomatically, for example, to suppress symptoms. It can be effected over a short period, be oriented over a medium term, or can be a long-term treatment, for example within the context of a maintenance therapy.

As used herein, the terms “prevent,” “preventing,” and “prevention” refer to a method of preventing the onset of a disease or condition and/or its attendant symptoms or barring a subject from acquiring a disease. As used herein, “prevent,” “preventing,” and “prevention” also include delaying the onset of a disease and/or its attendant symptoms and reducing a subject’s risk of acquiring a disease. The terms “prevent,” “preventing” and “prevention” may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition.

The term “therapeutically effective amount” or “effective dose” as used herein refers to an amount of the active ingredient(s) that is(are) sufficient, when administered by a method of the disclosure, to efficaciously deliver the active ingredient(s) for the treatment of condition or disease of interest to a subject in need thereof. In the case of a cancer or other proliferation disorder, the therapeutically effective amount of the agent may reduce (i.e., retard to some extent or stop) unwanted cellular proliferation; reduce the number of cancer cells; reduce the tumor size; inhibit (i.e., retard to some extent or stop) cancer cell infiltration into peripheral organs; inhibit (i.e., retard to some extent or stop) tumor metastasis; inhibit, to some extent, tumor growth; and/or relieve, to some extent, one or more of the symptoms associated with the cancer. To the extent the administered compound or composition prevents growth and/or kills existing cancer cells, it may be cytostatic and/or cytotoxic.

The term “container” means any receptacle and closure therefore suitable for storing, shipping, dispensing, and/or handling a pharmaceutical product.

The term “insert” means information accompanying a pharmaceutical product that provides a description of how to administer the product, along with the safety and efficacy data required to allow the physician, pharmacist, and patient to make an informed decision regarding use of the product. The package insert generally is regarded as the “label” for a pharmaceutical product.

“Concurrent administration,” “administered in combination,” “simultaneous administration,” and similar phrases mean that two or more agents are administered concurrently to the subject being treated. By “concurrently,” it is meant that each agent is administered either simultaneously or sequentially in any order at different points in time. However, if not administered simultaneously, it is meant that they are administered to a subject in a sequence and sufficiently close in time so as to provide the desired therapeutic effect and can act in concert. For example, a Compound of the Disclosure can be administered at the same time or sequentially in any order at different points in time as a second therapeutic agent. A Compound of the Disclosure and the second therapeutic agent can be administered separately, in any appropriate form and by any suitable route. When a Compound of the Disclosure and the second therapeutic agent are not administered concurrently, it is understood that they can be administered in any order to a subject in need thereof. For example, a Compound of the Disclosure can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent treatment modality (e.g., radiotherapy), to a subject in need thereof. In various embodiments, a Compound of the Disclosure and the second therapeutic agent are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one embodiment, the components of the combination therapies are administered at about 1 minute to about 24 hours apart.

The use of the terms “a”, “an”, “the”, and similar referents in the context of describing the disclosure (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure and is not a limitation on the scope of the disclosure unless otherwise claimed. No language in the specification should be construed as indicating any non-claimed element as essential to the practice of the disclosure.

The term “halo” as used herein by itself or as part of another group refers to —Cl, —F, —Br, or —I. In one emobodiment, the halo is —Cl or —F. In another embodiment, the halo is —Cl.

The term “alkyl” as used herein by itself or as part of another group refers to a straight- or branched-chain aliphatic hydrocarbon containing one to twelve carbon atoms, i.e., a C₁-C₁₂ alkyl, or the number of carbon atoms designated, e.g., a C₁ alkyl such as methyl, a C₂ alkyl such as ethyl, etc. In one embodiment, the alkyl is a C₁-C₁₀ alkyl. In another embodiment, the alkyl is a C₁-C₆ alkyl. In another embodiment, the alkyl is a C₁-C₄ alkyl. In another embodiment, the alkyl is a C₁-C₃ alkyl, i.e., methyl, ethyl, propyl, or isopropyl. Non-limiting exemplary C₁-C₁₂ alkyl groups include methyl, ethyl, propyl, isopropyl, butyl, sec-butyl, tert-butyl, iso-butyl, 3-pentyl, hexyl, heptyl, octyl, nonyl, and decyl.

The term “haloalkyl” as used herein by itself or as part of another group refers to an alkyl group substituted by one or more fluorine, chlorine, bromine, and/or iodine atoms. In one embodiment, the alkyl is substituted by one, two, or three fluorine and/or chlorine atoms. In another embodiment, the alkyl is substituted by one, two, or three fluorine atoms. In another embodiment, the alkyl is a C₁-C₆ alkyl. In another embodiment, the alkyl is a C₁-C₄ alkyl. In another embodiment, the alkyl group is a C₁ or C₂ alkyl. Non-limiting exemplary haloalkyl groups include fluoromethyl, difluoromethyl, trifluoromethyl, pentafluoroethyl, 1,1-difluoroethyl, 2,2-difluoroethyl, 2,2,2-trifluoroethyl, 3,3,3-trifluoropropyl, 4,4,4-trifluorobutyl, and trichloromethyl groups.

The term “cycloalkyl” as used herein by itself or as part of another group refers to saturated and partially unsaturated, e.g., containing one or two double bonds, monocyclic, bicyclic, or tricyclic aliphatic hydrocarbons containing three to twelve carbon atoms, i.e., a C₃₋₁₂ cycloalkyl, or the number of carbons designated, e.g., a C₃ cycloalkyl such a cyclopropyl, a C₄ cycloalkyl such as cyclobutyl, etc. In one embodiment, the cycloalkyl is bicyclic, i.e., it has two rings. In another embodiment, the cycloalkyl is monocyclic, i.e., it has one ring. In another embodiment, the cycloalkyl is a C₃₋₈ cycloalkyl. In another embodiment, the cycloalkyl is a C₃₋₆ cycloalkyl, i.e., cyclopropyl, cyclobutyl, cyclopentyl, or cyclohexyl. In another embodiment, the cycloalkyl is a C₅ cycloalkyl, i.e., cyclopentyl or cyclopentenyl. In another embodiment, the cycloalkyl is a C₆ cycloalkyl, i.e., cyclohexyl or cyclohexenyl. Non-limiting exemplary C₃-₁₂ cycloalkyl groups include cyclopropyl, cyclobutyl, cyclopentyl, cyclohexyl, cycloheptyl, cyclooctyl, norbornyl, decalin, adamantyl, cyclohexenyl, and spiro[3.3]heptane.

The present disclosure encompasses any of the Compounds of the Disclosure being isotopically-labelled (i.e., radiolabeled) by having one or more atoms replaced by an atom having a different atomic mass or mass number. Examples of isotopes that can be incorporated into the disclosed compounds include isotopes of hydrogen, carbon, nitrogen, oxygen, phosphorous, fluorine and chlorine, such as ²H (or deuterium (D)), ³H, ¹¹C_{,} ¹³C, ¹⁴C, ¹⁵N, ¹⁸O, ¹⁷O, ³¹P, ³²P, ³⁵S, ¹⁸F, and ³⁶Cl, respectively, e.g., ³H, ¹¹C, and ¹⁴C. In one embodiment, provided is a composition wherein substantially all of the atoms at a position within the Compound of the Disclosure are replaced by an atom having a different atomic mass or mass number. In another embodiment, provided is a composition wherein a portion of the atoms at a position within the Compound of the disclosure are replaced, i.e., the Compound of the Disclosure is enriched at a position with an atom having a different atomic mass or mass number.” Isotopically-labelled Compounds of the Disclosure can be prepared by methods known in the art.

Compounds of the Disclosure may contain one or more asymmetric carbon atoms and may thus give rise to enantiomers, diastereomers, and other stereoisomeric forms. The present disclosure encompasses the use of all such possible forms, as well as their racemic and resolved forms and mixtures thereof. The individual enantiomers can be separated according to methods known in the art in view of the present disclosure. When the compounds described herein contain olefinic double bonds or other centers of geometric asymmetry, and unless specified otherwise, it is intended that they include both E and Z geometric isomers. All tautomers are also encompassed by the present disclosure.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” or “asymmetric carbon atom” refers to a carbon atom to which four different groups are attached.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction.

The term “racemic” refers to a mixture of equal parts of enantiomers and which mixture is optically inactive. In one embodiment, Compounds of the Disclosure are racemic.

The term “absolute configuration” refers to the spatial arrangement of the atoms of a chiral molecular entity (or group) and its stereochemical description, e.g., R or S.

The stereochemical terms and conventions used in the specification are meant to be consistent with those described in Pure & Appl. Chem 68:2193 (1996), unless otherwise indicated.

The term “enantiomeric excess” or “ee” refers to a measure for how much of one enantiomer is present compared to the other. For a mixture of R and S enantiomers, the percent enantiomeric excess is defined as | R - S | * 100, where R and S are the respective mole or weight fractions of enantiomers in a mixture such that R + S = 1. With knowledge of the optical rotation of a chiral substance, the percent enantiomeric excess is defined as ([α]_{obs}/[α]ₘₐₓ)*100, where [α]_{obs} is the optical rotation of the mixture of enantiomers and [α]ₘₐₓ is the optical rotation of the pure enantiomer. Determination of enantiomeric excess is possible using a variety of analytical techniques, including NMR spectroscopy, chiral column chromatography or optical polarimetry.

The term “about,” as used herein, includes the recited number ± 10%. Thus, “about 10” means 9 to 11.

### EXAMPLES

General Information. All commercial reagents and solvents were used as supplied without further purification. Proton nuclear magnetic resonance (¹H NMR) and carbon nuclear magnetic resonance (¹³C NMR) spectroscopy were performed on Bruker Advance 400 NMR spectrometers. ¹H NMR spectra are reported in parts per million (ppm) downfield from tetramethylsilane (TMS). All ¹³C NMR spectra are reported in ppm and obtained with ¹H decoupling. In the spectral data reported, the format (8) chemical shift (multiplicity, J values in Hz, integration) was used with the following abbreviations: s = singlet, brs = broad singlet, d = doublet, t = triplet, q = quartet, m = multiplet. Electrospray ionization (ESI) mass spectral (MS) analysis was performed on a Thermo Scientific LCQ Fleet mass spectrometer. The final products were purified by reverse-phase HPLC (RP-HPLC) with solvent A (0.1% of TFA in water) and solvent B (0.1% of TFA in CH₃CN) as eluents with a flow rate of 45 mL/min. All final compounds have purity ≥95% as determined by Waters ACQUITY ultra-performance liquid chromatography (UPLC) using a reverse-phase column (SunFire, C18, 5 µm, 4.6 × 150 mm2) and a solvent gradient of solvent A (H₂O with 0.1% of TFA) and solvent B (CH₃CN with 0.1% of TFA).

### Example 1

### N1-(Amino-5-(4-Amino-4-Methylpiperidin-1-yl)Pyrazin-2-Yl)thio)-2-Chlorophenyl)-N3-(((S)-1-((2S,4R)-4-Hydroxy-2-(((S)-1-(4-(4-Methylthiazol-5-yl)Phenyl)ethyl)Carbamoyl)Pyrrolidin-1-yl)-3,3-Dimethyl-1-Oxobutan-2-yl)Amino)-3-Oxopropyl)Malonamide

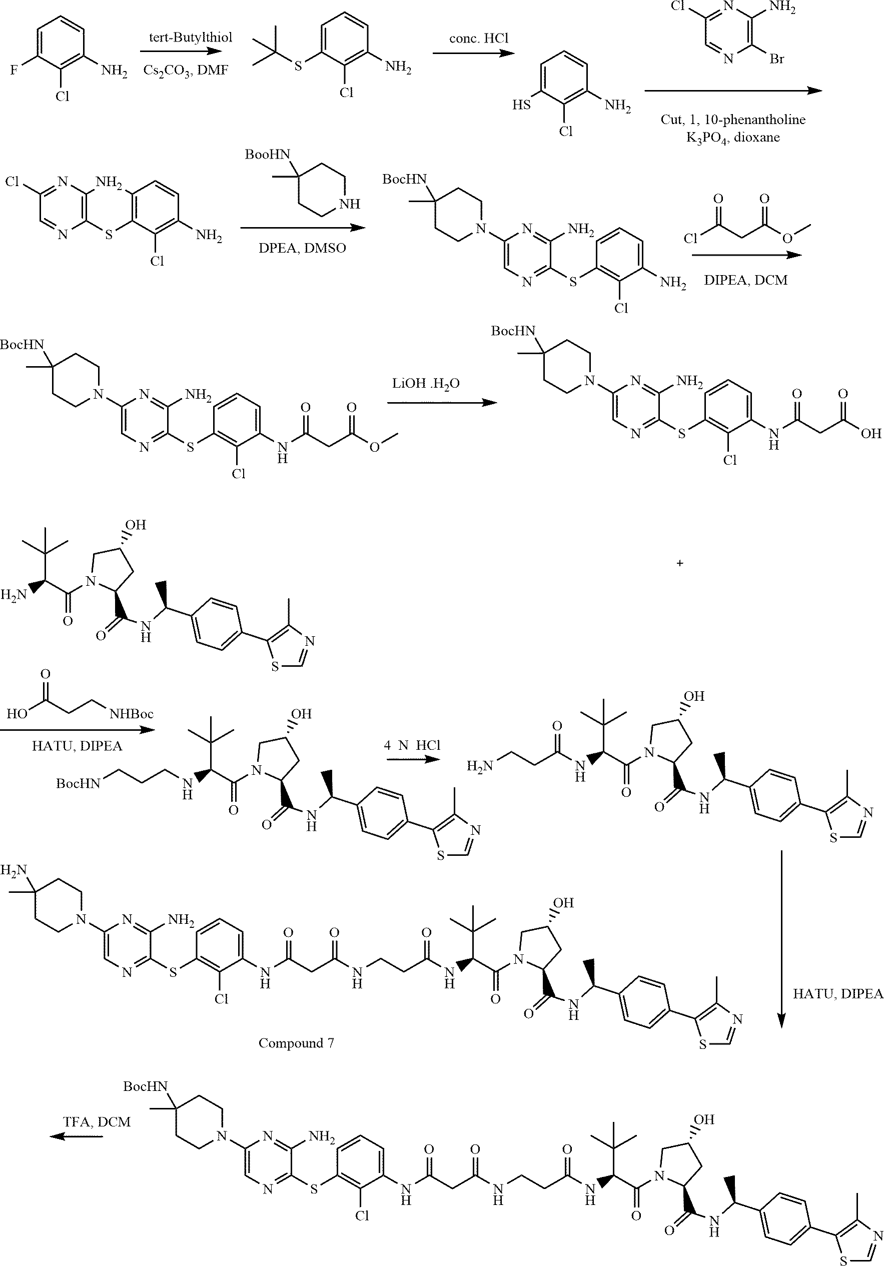

Step 1: To a solution of 2-chloro-3-fluoroaniline (15.0 g, 103.1 mmole) and tert-butylthiol ( 41 mL, 32.8 g, 360.9 mmole, 3.5 equiv) in anhydrous DMF (160 mL) was added cesium carbonate ( 84.0 g, 257.8 mmole, 2.5 equiv) at room temperature. The reaction mixture was heated to 120° C. and stirred for 36 h under N₂ protection. After cooling down, the reaction mixture was diluted with ethyl acetate (300 mL), washed with H₂O, brine, dried over Na₂SO₄ and evaporated in vacuo to afford crude 3-(tert-butylthio)-2-chloroaniline ( 19.8 g, 91.8 mmole) which was used directly for the next step. UPLC-MS (ESI⁺) calculated for C₁₀H₁₅ClNS [M+1]⁺: 216.06, found 216.10.

Step 2: 3-(tert-butylthio)-2-chloroaniline (19.5 g, 90.4 mmole) was suspended in conc. HC1 (170 mL) and the reaction mixture was vigorously stirred for 6 h at 80° C. After cooling down, the suspension was filtered, the white solids were washed with cold conc. HC1 (15 mL) and hexane (30 mL), and dried under reduced pressure to give 3-amino-2-chlorobenzenethiol hydrochloride (13.8 g, 70.5 mmole). UPLC-MS (ESI⁺) calculated for C₆H₇ClNS [M+1]⁺: 160.00, found 160.10.

Step 3: Potassium phosphate (20.7 g, 97.6 mmole, 2.6 equiv) was added to a solution of 3-bromo-6-chloropyrazin-2-amine (7.8 g, 37.5 mmole, 1.0 equiv) and 3-amino-2-chlorobenzenethiol hydrochloride (9.6 g, 48.8 mmole, 1.3 equiv) in dioxane (120 mL). The reaction mixture was degassed and stirred at rt for 15 mins, then CuI (1.4 g, 7.5 mmole, 0.2 equiv) and 1, 10-phenanthroline (2.7 g, 15.0 mmole, 0.4 equiv) were added. After degassed three times, the mixture was stirred at 90° C. under dry nitrogen for 16 h. The reaction mixture was cooled to rt, diluted with ethyl acetate (150 mL), and it was filtered through a pad of Celite followed by EtOAc wash. The volatiles were rreoved under reduced pressure and the residue was purified by silica gel chromatography eluting with 0-10% MeOH/DCM to afford 3-((3-amino-2-chlorophenyl)thio)-6-chloropyrazin-2-amine (6.6 g, 22.8 mmole, 61%) as yellow solid. ¹H NMR (400 MHz, DMSO-d₆) δ (ppm) 7.70 (s, 1H), 7.01-6.97 (m, 3H), 6.79 (dd, J= 8.0 Hz, J= 1.2 Hz, 1H), 6.49 (dd, J = 7.6 Hz, J = 1.6 Hz, 1H), 5.51 (s, 2H); UPLC-MS (ESI⁺) calculated for C₁₀H₉Cl₂N₄S [M+1]⁺: 286.99, found 287.05.

Step 4: To the solution of 3-((3-amino-2-chlorophenyl)thio)-6-chloropyrazin-2-amine ( 3.6 g, 12.5 mmol, 1.0 equiv) and tert-butyl (4-methylpiperidin-4-yl)carbamate (5.4 g, 25.0 mmol, 2.0 equiv) in DMSO (50 mL) was added DIPEA (6.5 mL, 4.9 g, 37.5 mmol, 3.0 equiv) (1 mL) at rt. The reaction mixture was allowed to warm to 100° C. and stirred for 2 h. After cooling down, it was poured onto ice-cold water (200 mL). After extracted with ethyl acetate (100 mL × 2), the combined organic layers were washed with water, brine, dried over Na₂SO₄ and evaporated under vacuum. The residue was purified by silica gelflash column chromatography with hexane:EtOAc (4:1-1:3) to afford tert-butyl (1-(6-amino-5-((3-amino-2-chlorophenyl)thio)pyrazin-2-yl)-4-methylpiperidin-4-yl as white solid (4.4 g, 9.5 mmol, 76% yield). ¹H NMR (400 MHz, DMSO-d₆) δ (ppm) 7.59 (s, 1H), 6.83 (t, J= 8.0 Hz, J= 1.2 Hz, 1H), 6.62 (s, 1H), 6.55 (dd, J= 8.0 Hz, J= 1.2 Hz, 1H), 5.99 (s, 2H); 5.81 (dd, J= 8.0 Hz, J= 1.6 Hz, 1H), 5.41 (s, 2H), 3.85-3.81 (m, 2H), 3.23-3.18 (m, 2H), 2.09-2.05 (m, 2H), 1.46-1.41 (m, 2H), 1.39 (s, 9H), 1.25 (s, 3H); UPLC-MS (ESI⁺) calculated for C₁₀H₉Cl₂N₄S [M+1]⁺: 286.99, found 287.05.

Step 5: To the solution of tert-butyl (1-(6-amino-5-((3-amino-2-chlorophenyl)thio)pyrazin-2-yl)-4-methylpiperidin-4-yl ( 3.4 g, 7.4 mmol, 1.0 equiv) and DIPEA (3.9 mL, 2.9 g, 22.2 mmol, 3.0 equiv) in DCM (50 mL) was added methyl 3-chloro-3-oxopropanoate (1.2 g, 8.9 mmol, 1.2 equiv) dropwise at 0° C. The reaction mixture was allowed to warm to rt and stirred for 1 h. It was poured onto aq. NaHCO₃ solution (50 mL). After extracted with DCM (30 mL × 2), the combined organic layers were washed with brine, dried over Na₂SO₄ and evaporated under vacuum. The residue was used in the next step without further purification. UPLC-MS (ESI⁺) calculated for C₂₅H₃₄ClN₆O₅S [M+1]⁺: 565.20, found 565.33.

Step 6: To a solution of residue obtained from up was dissolved in THF/MeOH/H₂O (15 mL/10 mL/ 5 mL) was added Lithium hydroxide monohydrate (932 mg, 22.2 mmole) at 0° C. The reaction mixture was allowed to warm to rt and stirred for 2 h. After quenched with 1 N HCl to pH~3, the resulting mixture was extracted with ethyl acetate (40 mL × 2). The organic layer was washed with brine, dried over anhydrous Na₂SO₄, and then concentrated under a reduced pressure. The residue was then purified by silica gel column chromatography eluting with 0-5% MeOH/DCM to give 3-((3-((3-amino-5-(4-((tert-butoxycarbonyl)amino)-4-methylpiperidin-1-yl)pyrazin-2-yl)thio)-2-chlorophenyl)amino)-3-oxopropanoic acid (2.5 g, 4.5 mmol, 61%, two steps) as an white-yellow solid. ¹H NMR (400 MHz, DMSO-d₆) δ (ppm) 12.72 (s, 1H), 9.85 (s, 1H), 7.62 (s, 1H), 7.58 (d, J= 7.6 Hz, 1H), 7.17 (t, J= 8.0 Hz, J= 1.2 Hz, 1H), 6.63 (s, 1H), 6.42 (dd, J = 8.0 Hz, J = 1.2 Hz, 1H), 6.11 (s, 2H); 5.81 (dd, J = 8.0 Hz, J = 1.6 Hz, 1H), 3.86-3.84 (m, 2H), 3.49 (s, 2H), 3.24-3.19 (m, 2H), 2.10-2.06 (m, 2H), 1.46-1.41 (m, 2H), 1.39 (s, 9H), 1.25 (s, 3H); UPLC-MS (ESI⁺) calculated for C₂₄H₃₂C1N₆O₅S [M+1]⁺: 551.18, found 551.25.

Step 7: HATU (295 mg, 0.77 mmol, 1.1 equiv) was added to a solution of (2S,4R)-1-((S)-2-amino-3,3-dimethylbutanoyl)-4-hydroxy-N-((S)-1-(4-(4-methylthiazol-5-yl)phenyl)ethyl)pyrrolidine-2-carboxamide (335 mg, 0.60 mmol, 1 equiv), 68 (125 mg, 0.66 mmol, 1.1 equiv), and DIEA (0.42 mL, 2.40 mmol, 4.0 equiv) in DMF (6 mL), and the resulting mixture was stirred at rt for 1 h. The solution was diluted with EtOAc and washed with H2O, saturated sodium bicarbonate aqueous solution, and brine and dried over sodium sulfate. After removal of the solvent in vacuo, the residue was purified by HPLC afford tert-butyl (3-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-3-oxopropyl)carbamate.

Step 8: To the solution of tert-butyl (3-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-3-oxopropyl)carbamate in DCM was added 4N HCl/dioxane and stirred for 1h at rt, then the solvent was removed to afford (2S,4R)-1-((S)-2-(3-aminopropanamido)-3,3-dimethylbutanoyl)-4-hydroxy-N-((S)-1-(4-(4-methylthiazol-5-yl)phenyl)ethyl)pyrrolidine-2-carboxamide as hydrochloride salt. ¹H NMR (400 MHz, DMSO-d₆) δ (ppm) 9.04 (s, 1H), 8.43 (d, J= 8.0 Hz, 1H), 8.23 (d, J= 8.8 Hz, 1H), 7.90 (s, 3H), 7.45-7.37 (m, 4H), 4.95-4.88 (m, 1H), 4.52 (d, J = 8.8 Hz, 1H), 4.44-4.40 (m, 1H), 3.65-3.59 (m, 1H), 2.98-2.93 (m, 2H), 2.63-2.58 (m, 2H), 2.46 (s, 3H), 2.07-1.99 (m, 1H), 1.82-1.76 (m, 1H), 1.37 (d, J= 7.2 Hz, 3H), 1.30-1.26 (m, 1H), 0.95 (s, 9H); UPLC-MS (ESI⁺) calculated for C₂₆H₃₈N₅O₄S [M+1]⁺: 516.26, found . 516.19

Step 9: HATU (25 mg, 0.066 mmol, 1.1 equiv) was added to a mixture of (2S,4R)-1-((S)-2-(3-aminopropanamido)-3,3-dimethylbutanoyl)-4-hydroxy-N-((S)-1-(4-(4-methylthiazol-5-yl)phenyl)ethyl)pyrrolidine-2-carboxamide hydrochloride (33 mg, 0.06 mmol, 1.0 equiv), 3-((3-((3-amino-5-(4-((tert-butoxycarbonyl)amino)-4-methylpiperidin-1-yl)pyrazin-2-yl)thio)-2-chlorophenyl)amino)-3-oxopropanoic acid (33 mg, 0.06 mmol, 1.0 equiv), and DIPEA (39 mg, 0.30 mmol, 5.0 equiv) in DMF (2 mL) at 0° C. under N2. The mixture was stirred at ambient temperature for 1 h. After quenched with water (8 mL) and extracted with ethyl acetate (5 mL × 3), the organic layers were washed with brine (15 mL), dried over anhydrous Na₂SO₄, and concentrated under a reduced pressure. The residue was dissolved in DCM (3 mL) was added trifluoracetic acid (1 mL) at 0° C. The reaction was stirred for 30 min, and the solvent was removed in vacuo. The residue was purified by reverse-phase chromatography over a C18 column to yield Compound 7 (37 mg, 58%) as a white powder. ¹H NMR (400 MHz, DMSO-d₆) δ (ppm) 10.31 (s, 1H), 9.00 (s, 1H), 8.39 (d, J= 8.0 Hz, 1H), 8.32-8.29 (m, 1H), 7.97 (brs, 3H), 7.80 (d, J= 7.2 Hz, 1H), 7.66 (s, 1H), 7.45-7.37 (m, 4H), 7.16 (t, J= 8.0 Hz, 1H), 6.40 (dd, J= 8.0 Hz, J= 1.2 Hz, 1H), 4.95-4.85 (m, 1H), 4.52 (d, J= 8.8 Hz, 1H), 4.43 (t, J= 8.0 Hz, 1H), 4.29 (s, 1H), 4.06-4.02 (m, 2H), 3.64-3.57 (m, 2H), 3.38 (s, 2H), 3.33-3.26 (m, 2H), 2.45 (s, 3H), 2.43-2.33 (m, 2H), 2.04-1.99 (m, 1H), 1.81-1.77 (m, 1H), 1.73-1.70 (m, 4H), 1.38 (s, 3H), 1.36 (s, 3H), 0.92 (s, 9H); UPLC-MS (ESI⁺) calculated for C₄₅H₅₉ClN₁₁O₆S₂ [M+1]⁺: 948.38, found 948.31.

The compounds of Table 4 were made using the procedure or modifications to the procedure as exemplified for Example 1.

TABLE 4

| Compound | MW | Observed [M+H]/z | ¹H NMR |
|---|---|---|---|
| 8 | 962.39 | 962.33 | |
| 9 | 976.41 | 976.45 | |
| 10 | 990.42 | 990.39 | |
| 11 | 1004.44 | 1004.37 | |
| 12 | 1018.46 | 1018.41 | |
| 13 | 1032.47 | 1032.42 | δ (ppm) 10.32 (s, 1H), 8.99 (s, 1H), 8.38 (d, J= 8.0 Hz, 1H), 8.26-8.23 (m, 1H), 7.97 (brs, 3H), 7.82-7.78 (m, 2H), 7.66 (s, 1H), 7.45-7.36 (m, 4H), 7.16 (t, J= 8.0 Hz, 1H), 6.40 (dd, J= 8.0 Hz, J= 0.8 Hz, 1H), 6.20 (brs, 1H), 4.95-4.88 (m, 1H), 4.51 (d, J= 8.8 Hz, 1H), 4.41 (t, J= 8.8 Hz, 1H), 4.27 (s, 1H), 4.05-4.02 (m, 2H), 3.63-3.57 (m, 2H), 3.38 (s, 2H), 3.33-3.26 (m, 2H), 3.12-3.07 (m, 2H), 2.45 (s, 3H), 2.28-2.21 (m, 1H), 2.13-1.98 (m, 2H), 1.82-1.70 (m, 5H), 1.47-1.40 (m, 4H), 1.38 (s, 3H), 1.36 (s, 3H), 1.28-1.17 (m, 8H), 0.93 (s, 9H) |
| 14 | 1046.49 | 1046.45 | δ (ppm) 10.33 (s, 1H), 9.00 (s, 1H), 8.39 (d, J= 8.0 Hz, 1H), 8.26-8.23 (m, 1H), 7.98 (brs, 3H), 7.83-7.78 (m, 2H), 7.67 (s, 1H), 7.47-7.33 (m, 4H), 7.15 (t, J= 8.0 Hz, 1H), 6.41 (dd, J= 8.0 Hz, J= 1.2 Hz, 1H), 6.21 (brs, 1H), 4.96-4.88 (m, 1H), 4.52 (d, J= 8.8 Hz, 1H), 4.42 (t, J= 8.0 Hz, 1H), 4.28 (s, 1H), 4.06-4.03 (m, 2H), 3.64-3.58 (m, 2H), 3.39 (s, 2H), 3.34-3.27 (m, 2H), 3.13-3.08 (m, 2H), 2.46 (s, 3H), 2.29-2.22 (m, 1H), 2.14-1.99 (m, 2H), 1.82-1.71 (m, 5H), 1.51-1.39 (m, 4H), 1.38 (s, 3H), 1.36 (s, 3H), 1.27-1.19 (m, 10H), 0.93 (s, 9H) |
| 15 | 1060.50 | 1060.51 | δ (ppm) 10.31 (s, 1H), 8.99 (s, 1H), 8.36 (d, J= 8.0 Hz, 1H), 8.24-8.22 (m, 1H), 7.95 (brs, 3H), 7.82-7.75 (m, 2H), 7.66 (s, 1H), 7.44-7.37 (m, 4H), 7.16 (t, J= 8.0 Hz, 1H), 6.41 (dd, J= 8.0 Hz, J= 1.2 Hz, 1H), 6.18 (brs, 2H), 4.95-4.88 (m, 1H), 4.51 (d, J= 8.8 Hz, 1H), 4.42 (t, J= 8.0 Hz, 1H), 4.28 (s, 1H), 4.05-4.02 (m, 2H), 3.64-3.57 (m, 2H), 3.38 (s, 2H), 3.33-3.26 (m, 2H), 3.12-2.97 (m, 2H), 2.45 (s, 3H), 2.28-2.21 (m, 1H), 2.13-1.98 (m, 2H), 1.82-1.70 (m, 5H), 1.50-1.40 (m, 4H), 1.38 (s, 3H), 1.36 (s, 3H), 1.29-1.15 (m, 12H), 0.93 (s, 9H) |
| 16 | 1074.52 | 1074.38 | δ (ppm) 10.30 (s, 1H), 8.98 (s, 1H), 8.36 (d, J= 8.0 Hz, 1H), 8.24-8.21 (m, 1H), 7.96 (brs, 3H), 7.83-7.75 (m, 2H), 7.66 (s, 1H), 7.44-7.37 (m, 4H), 7.16 (t, J= 8.0 Hz, 1H), 6.41 (dd, J= 8.0 Hz, J= 1.2 Hz, 1H), 6.15 (brs, 2H), 4.95-4.88 (m, 1H), 4.51 (d, J= 8.8 Hz, 1H), 4.42 (t, J= 8.0 Hz, 1H), 4.28 (s, 1H), 4.05-4.02 (m, 2H), 3.64-3.57 (m, 2H), 3.38 (s, 2H), 3.33-3.26 (m, 2H), 3.12-3.07 (m, 2H), 2.45 (s, 3H), 2.29-2.21 (m, 1H), 2.13-1.98 (m, 2H), 1.83-1.70 (m, 5H), 1.50-1.40 (m, 4H), 1.38 (s, 3H), 1.36 (s, 3H), 1.28-1.16 (m, 14H), 0.93 (s, 9H) |
| 17 | 1074.52 | 1074.47 | δ (ppm) 9.54 (s, 1H), 8.98 (s, 1H), 8.36 (d, J= 8.0 Hz, 1H), 7.97 (brs, 3H), 7.84-7.75 (m, 2H), 7.66 (s, 1H), 7.49-7.37 (m, 4H), 7.14 (t, J= 8.0 Hz, 1H), 6.43 (dd, J = 8.0 Hz, J = 1.2 Hz, 1H), 6.18 (brs, 2H), 4.95-4.88 (m, 1H), 4.52 (d, J= 8.8 Hz, 1H), 4.42 (t, J= 8.0 Hz, 1H), 4.27 (s, 1H), 4.05-4.02 (m, 2H), 3.64-3.57 (m, 2H), 3.33-3.26 (m, 2H), 3.05-3.00 (m, 2H), 2.63-2.59 (m, 2H), 2.45 (s, 3H), 2.41-2.37 (m, 2H), 2.28-2.21 (m, 1H), 2.13-1.98 (m, 2H), 1.82-1.70 (m, 5H), 1.52-1.40 (m, 4H), 1.38 (s, 3H), 1.36 (s, 3H), 1.28-1.17 (m, 12H), 0.93 (s, 9H) |
| 18 | 1088.53 | 1088.49 | δ (ppm) 9.54 (s, 1H), 8.98 (s, 1H), 8.36 (d, J= 8.0 Hz, 1H), 7.95 (brs, 3H), 7.84-7.75 (m, 2H), 7.66 (s, 1H), 7.48-7.37 (m, 5H), 7.14 (t, J= 8.0 Hz, 1H), 6.43 (dd, J = 8.0 Hz, J = 1.2 Hz, 1H), 6.18 (brs, 2H), 4.95-4.88 (m, 1H), 4.51 (d, J= 8.8 Hz, 1H), 4.42 (t, J= 8.0 Hz, 1H), 4.28 (s, 1H), 4.05-4.02 (m, 2H), 3.63-3.57 (m, 2H), 3.33-3.26 (m, 2H), 3.05-3.00 (m, 2H), 2.63-2.59 (m, 2H), 2.45 (s, 3H), 2.41-2.37 (m, 2H), 2.28-2.21 (m, 1H), 2.13-1.98 (m, 2H), 1.82-1.70 (m, 5H), 1.52-1.41 (m, 4H), 1.37 (s, 3H), 1.36 (s, 3H), 1.27-1.16 (m, 14H), 0.93 (s, 9H) |
| 19 | 1102.55 | 1102.48 | δ (ppm) 9.52 (s, 1H), 8.99 (s, 1H), 8.38 (d, J= 8.0 Hz, 1H), 7.96 (brs, 3H), 7.80-7.75 (m, 2H), 7.67 (s, 1H), 7.45-7.37 (m, 4H), 7.15 (t, J= 8.0 Hz, 1H), 6.43 (dd, J = 8.0 Hz, J = 1.2 Hz, 1H), 6.19 (brs, 2H), 4.95-4.89 (m, 1H), 4.51 (d, J= 8.8 Hz, 1H), 4.41 (t, J= 8.0 Hz, 1H), 4.27 (s, 1H), 4.06-4.02 (m, 2H), 3.64-3.57 (m, 2H), 3.33-3.26 (m, 2H), 3.04-2.99 (m, 2H), 2.45 (s, 3H), 2.39-2.35 (m, 2H), 2.28-2.21 (m, 1H), 2.14-1.98 (m, 4H), 1.82-1.70 (m, 6H), 1.47-1.41 (m, 4H), 1.37 (s, 3H), 1.36 (s, 3H), 1.27-1.18 (m, 16H), 0.93 (s, 9H) |
| 20 | 1080.46 | 1080.21 | δ (ppm) 10.29 (s, 1H), 9.00 (s, 1H), 8.45 (d, J= 8.0 Hz, 1H), 8.38 (t, J= 5.2 Hz, 1H), 8.07 (brs, 3H), 7.80 (dd, J= 8.0 Hz, J= 1.6 Hz, 1H), 7.66 (s, 1H), 7.45-7.33 (m, 5H), 7.15 (t, J= 8.0 Hz, 1H), 6.40 (dd, J= 8.0 Hz, J= 1.2 Hz, 1H), 6.21 (brs, 1H), 4.94-4.87 (m, 1H), 4.54 (d, J= 8.8 Hz, 1H), 4.44 (t, J= 8.0 Hz, 1H), 4.28 (s, 1H), 4.05-3.92 (m, 4H), 3.63-3.54 (m, 10H), 3.48-3.42 (m, 4H), 3.35-3.27 (m, 4H), 2.45 (s, 3H), 2.39-2.35 (m, 2H),2.08-1.99 (m, 1H), 1.80-1.69 (m, 5H), 1.38 (s, 3H), 1.36 (s, 3H), 0.94 (s, 9H) |
| 21 | 1094.47 | 1094.35 | δ (ppm) 10.28 (s, 1H), 9.00 (s, 1H), 8.40-8.36 (m, 2H), 8.06 (brs, 3H), 7.84 (d, J= 8.8 Hz, 1H), 7.80 (dd, J= 8.0 Hz, J= 1.6 Hz, 1H), 7.66 (s, 1H), 7.45-7.33 (m, 4H), 7.16 (t, J= 8.0 Hz, 1H), 6.40 (dd, J= 8.0 Hz, J= 1.2 Hz, 1H), 6.19 (brs, 1H), 4.95-4.88 (m, 1H), 4.52 (d, J= 8.8 Hz, 1H), 4.42 (t, J= 8.0 Hz, 1H), 4.27 (s, 1H), 4.05-4.00 (m, 2H), 3.63-3.55 (m, 4H), 3.51-3.42 (m, 12H), 3.34-3.26 (m, 4H), 2.57-2.52 (m, 1H), 2.45 (s, 3H), 2.40-2.32 (m, 3H), 2.07-1.99 (m, 1H), 1.80-1.69 (m, 5H), 1.38 (s, 3H), 1.36 (s, 3H), 0.94 (s, 9H) |
| 22 | 1124.48 | 1124.35 | δ (ppm) 10.29 (s, 1H), 9.00 (s, 1H), 8.45 (d, J= 8.0 Hz, 1H), 8.38-8.36 (m, 1H), 7.96 (brs, 3H), 7.80 (dd, J= 8.0 Hz, J= 1.6 Hz, 1H), 7.66 (s, 1H), 7.46-7.33 (m, 5H), 7.16 (t, J= 8.0 Hz, 1H), 6.39 (dd, J= 8.0 Hz, J= 1.2 Hz, 1H), 6.21 (brs, 1H), 4.94-4.87 (m, 1H), 4.54 (d, J= 8.8 Hz, 1H), 4.43 (t, J= 8.0 Hz, 1H), 4.28 (s, 1H), 4.05-4.02 (m, 2H), 3.96 (s, 2H), 3.62-3.53 (m, 14H), 3.47-3.42 (m, 4H), 3.33-3.26 (m, 4H), 2.45 (s, 3H), 2.38-2.33 (m, 2H), 2.07-2.02 (m, 1H), 1.87-1.70 (m, 5H), 1.38 (s, 3H), 1.36 (s, 3H), 0.94 (s, 9H) |
| 26 | 1115.54 | 1115.29 | δ (ppm) 9.67 (s, 1H), 9.57 (s, 1H), 8.99 (s, 1H), 8.38 (d, J= 8.0 Hz, 1H), 7.97 (brs, 3H), 7.79 (d, J= 8.8 Hz, 1H), 7.66 (s, 1H), 7.47-7.35 (m, 4H), 7.15 (t, J= 8.0 Hz, 1H), 6.41 (dd, J= 8.0 Hz, J = 1.2 Hz, 1H), 6.19 (brs, 1H), 4.95-4.89 (m, 1H), 4.51 (d, J= 8.8 Hz, 1H), 4.45-4.39 (m, 2H), 4.28 (s, 1H), 4.14-4.02 (m, 3H), 3.63-3.56 (m, 2H), 3.50-3.45 (m, 2H), 3.40-3.26 (m, 3H), 3.10-3.05 (m, 3H), 2.96-2.83 (m, 2H), 2.72-2.63 (m, 4H), 2.45 (s, 3H), 2.29-2.20 (m, 1H), 2.13-1.99 (m, 2H), 1.82-1.70 (m, 5H), 1.69-1.58 (m, 2H), 1.53-1.42 (m, 2H), 1.38 (s, 3H), 1.36 (s, 3H), 1.30-1.19 (m, 8H), 0.93 (s, 9H) |
| 27 | 1129.56 | 1129.38 | δ (ppm) 9.73 (s, 1H), 9.57 (s, 1H), 8.99 (s, 1H), 8.38 (d, J= 8.0 Hz, 1H), 7.99 (brs, 3H), 7.79 (d, J= 8.8 Hz, 1H), 7.66 (s, 1H), 7.47-7.35 (m, 4H), 7.14 (t, J= 8.0 Hz, 1H), 6.42 (dd, J= 8.0 Hz, J = 1.2 Hz, 1H), 6.19 (brs, 1H), 4.95-4.88 (m, 1H), 4.51 (d, J= 8.8 Hz, 1H), 4.46-4.39 (m, 2H), 4.28 (s, 1H), 4.14-4.02 (m, 3H), 3.63-3.56 (m, 2H), 3.50-3.45 (m, 2H), 3.37-3.26 (m, 3H), 3.10-3.03 (m, 3H), 2.97-2.84 (m, 2H), 2.72-2.61 (m, 4H), 2.45 (s, 3H), 2.29-2.21 (m, 1H), 2.13-1.99 (m, 2H), 1.82-1.70 (m, 5H), 1.69-1.57 (m, 2H), 1.52-1.41 (m, 2H), 1.38 (s, 3H), 1.36 (s, 3H), 1.31-1.20 (m, 10H), 0.93 (s, 9H) |
| 28 | 1124.50 | 1124.56 | δ (ppm) 9.84 (s, 1H), 9.60 (s, 1H), 8.99 (s, 1H), 8.38 (d, J= 8.0 Hz, 1H), 7.95 (brs, 3H), 7.80 (d, J= 8.8 Hz, 1H), 7.66 (s, 1H), 7.49-7.34 (m, 7H), 7.14 (t, J= 8.0 Hz, 1H), 6.87-6.82 (m, 2H), 6.42 (dd, J = 8.0 Hz, J = 1.2 Hz, 1H), 6.19 (brs, 1H), 4.95-4.88 (m, 1H), 4.52 (d, J= 8.8 Hz, 1H), 4.45-4.40 (m, 1H), 4.27 (s, 1H), 4.05-4.02 (m, 2H), 3.91-3.88 (m, 2H), 3.63-3.57 (m, 2H), 3.33-3.26 (m, 2H), 3.17-3.08 (m, 1H), 2.73-2.69 (m, 2H), 2.63-2.59 (m, 2H), 2.45 (s, 3H), 2.29-2.22 (m, 1H), 2.14-1.98 (m, 2H), 1.82-1.64 (m, 6H), 1.54-1.44 (m, 2H), 1.52-1.41 (m, 2H), 1.38 (s, 3H), 1.36 (s, 3H), 1.31-1.15 (m, 6H), 0.93 (s, 9H) |
| 29 | 1124.50 | 1124.47 | δ (ppm) 9.56 (s, 1H), 8.99 (s, 1H), 8.35 (d, J= 8.0 Hz, 1H), 8.02 (brs, 3H), 7.94-7.91 (m, 1H), 7.86 (d, J= 8.4 Hz, 1H), 7.80 (d, J= 8.8 Hz, 1H), 7.66 (s, 1H), 7.46-7.36 (m, 5H), 7.14 (t, J= 8.0 Hz, 1H), 6.87-6.82 (m, 2H), 6.43 (dd, J= 8.0 Hz, J= 1.2 Hz, 1H), 6.17 (brs, 2H), 4.95-4.87 (m, 1H), 4.52 (d, J= 8.8 Hz, 1H), 4.43-4.37 (m, 1H), 4.23 (s, 1H), 4.05-4.01 (m, 2H), 3.67-3.56 (m, 3H), 3.47-3.27 (m, 5H), 3.10-3.05 (m, 8H), 2.65-2.61 (m, 2H), 2.45 (s, 3H), 2.42-2.39 (m, 2H), 2.30-2.27 (m, 1H), 2.22-2.15 (m, 1H), 2.07-1.99 (m, 1H), 1.83-1.71 (m, 5H), 1.63-1.49 (m, 6H), 1.48-1.40 (m, 2H), 1.38 (s, 3H), 1.36 (s, 3H), 0.94 (s, 9H) |
| 30 | 991.69 | 991.52 | |
| 31 | 1005.72 | 1005.61 | |
| 32 | 1005.72 | 1005.61 | |
| 33 | 1019.74 | 1019.62 | |
| 34 | 1019.74 | 1019.81 | |
| 35 | 1033.77 | 1033.60 | |
| 36 | 1032.47 | 1032.35 | |
| 37 | 1046.49 | 1046.37 | |
| 38 | 1036.43 | 1036.31 | |
| 39 | 1050.45 | 1050.34 | |
| 40 | 1088.53 | 1088.48 | |
| 41 | 1102.55 | 1102.51 | |
| 42 | 1120.49 | 1120.41 | |

### Example 2

### (2R,4S)((R)-2-(11-(4-(2-((3-((3-Amino-5-(4-Amino-4-Methylpiperidinyl)Pyrazin-2-yl)Thio)-2-Chlorophenyl)amino)-2-Oxoethyl)Piperazin-1 -yl)Undecanamido)-3,3-Dimethylbutanoyl)-4-Hydroxy-N-((R)(4-(4-Methylthiazol-5-yl)Phenyl)ethyl)Pyrrolidine-2-Carboxamide

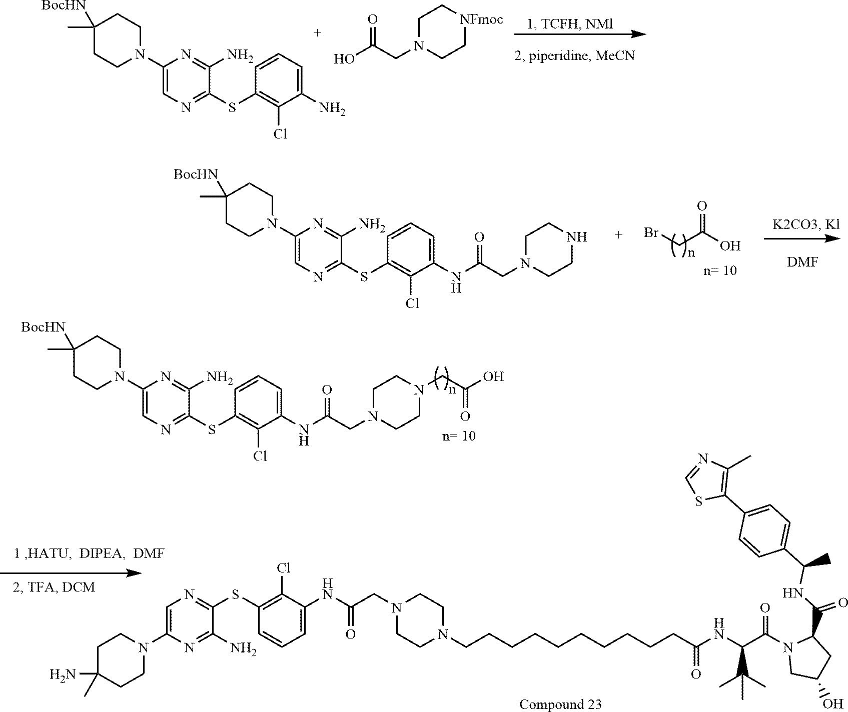

Step 1: tert-Butyl (1-(6-amino-5-((3-amino-2-chlorophenyl)thio)-pyrazin-2-yl)-4-methylpiperidin-4-yl)carbamate (930 mg, 2.0 mmol, 1.0 equiv), 2-(4-(((9H-fluoren-9-yl)methoxy)carbonyl)piperazin-1-yl)acetic acid (806 mg, 2.2 mmol, 1.1 equiv) and N-methylimidazole (574 mg, 7.0 mmol, 3.5 equiv) were dissolved in MeCN (8.0 mL) and THF (8.0 mL), then TCFH ( 730 mg, 2.6 mmol, 1.3 equiv) was added in a single portion. The reaction was stirred until complete, judged by LC-MS. The reaction was then diluted with EtOAc (30 mL) and water (30 mL). The layers were separated and washed with water, dried with Na₂SO₄, filtered and concentrated, the residue was dissolved in piperidine (2 mL) and MeCN (10 mL), after stirring at rt for 2 h and the solvent was removed under vacuum and the residue was purified by pre-HPLC to afford tert-butyl (1-(6-amino-5-((2-chloro-3-(2-(piperazin-1-yl)acetamido)phenyl)thio)pyrazin-2-yl)-4-methylpiperidin-4-yl)carbamate as a white solid (566 mg, 48% yield). ¹H NMR (400 MHz, DMSO-d₆) δ (ppm) 10.00 (s, 1H), 8.01 (dd, J= 8.0 Hz, J= 1.2 Hz, 1H), 7.62 (s, 1H), 7.19 (t, J= 8.0 Hz, 1H), 6.63 (brs, 1H), 6.39 (dd, J= 8.0 Hz, J= 1.2 Hz, 1H), 6.10 (brs, 1H), 3.37-3.29 (m, 2H), 3.27-3.17 (m, 2H), 3.16 (s, 1H), 2.87 (t, J= 4.8 Hz, 2H), 2.58-2.51 (m, 2H), 2.13-2.09 (m, 2H), 1.47-1.42 (m, 2H), 1.39 (s, 9H), 1.25 (s, 9H); UPLC-MS (ESI⁺) calculated for C₂₇H₄₀ClN₈O₃S [M+1] ⁺: 591.26, found. 591.21.

Step 2: K₂CO₃ (3.0 equiv) and KI (1.2 equiv) were added to a solution of the intermediate tert-butyl (1-(6-amino-5-((2-chloro-3-(2-(piperazin-1-yl)acetamido)phenyl)thio)pyrazin-2-yl)-4-methylpiperidin-4-yl)carbamate (118 mg, 0.2 mmol) and 11-bromoundecanoic acid (1.2 equiv) in DMF (5.0 mL). After stirring the mixture 12 h at 60° C., the reaction was diluted with EtOAc (30 mL) and water (30 mL). The layers were separated and washed with water, dried with Na₂SO₄, filtered and concentrated, the residue was purified by pre-HPLC to afford 11-(4-(2-((3-((3-amino-5-(4-((tert-butoxycarbonyl)amino)-4-methylpiperidin- 1 -yl)pyrazin-2-yl)thio)-2-chlorophenyl)amino)-2-oxoethyl)piperazin-1-yl)undecanoic acid as a white solid (71 mg, 46% yield). ¹H NMR (400 MHz, DMSO-d₆) δ (ppm) 9.80 (s, 1H), 9.44 (brs, 1H), 7.81 (d, J= 8.0 Hz, 1H), 7.62 (s, 1H), 7.20 (t, J= 8.0 Hz, 1H), 6.64 (brs, 1H), 6.44 (dd, J= 8.0 Hz, J= 1.2 Hz, 1H), 6.09 (brs, 1H), 3.87-3.83 (m, 2H), 3.55-3.50 (m, 2H), 3.38 (s, 1H), 3.25-3.20 (m, 2H), 3.16-3.03 (m, 6H), 2.73-2.65 (m, 2H), 2.19 (t, J= 7.6 Hz, 2H), 2.13-2.06 (m, 2H), 1.66-1.58 (m, 2H), 1.53-1.45 (m, 4H), 1.39 (s, 3H), 1.32-1.24 (m, 15H); UPLC-MS (ESI⁺) calculated for C₃₈H₆₀ClN₈O₅S [M+1]⁺: 775.41, found. 775.32.

Step 3: To the solution of 11-(4-(2-((3-((3-amino-5-(4-((tert-butoxycarbonyl)amino)-4-methylpiperidin- 1 -yl)pyrazin-2-yl)thio)-2-chlorophenyl)amino)-2-oxoethyl)piperazin-1-yl)undecanoic acid (65 mg, 0.084 mmole) and (2R,4S)-1-((R)-2-amino-3,3-dimethylbutanoyl)-4-hydroxy-N-((R)-1-(4-(4-methylthiazol-5-yl)phenyl)ethyl)pyrrolidine-2-carboxamide (42 mg, 0.095 mmole, 1.1 eq) in DMF was added DIPEA (44 mg, 0.336 mmole, 4.0 eq) and HATU (36 mg 0.095 mmole, 1.1 eq). The mixture was stirred at ambient temperature for 1 h. After quenched with water (8 mL) and extracted with ethyl acetate (5 mL × 3), the organic layers were washed with brine (15 mL), dried over anhydrous Na₂SO₄, and concentrated under a reduced pressure. The residue was dissolved in DCM (3 mL) was added trifluoracetic acid (1 mL) at 0° C. The reaction was stirred for 30 min, and the solvent was removed in vacuo. The residue was purified by reverse-phase chromatography over a C18 column to yield Compound 23 as a white powder. ¹H NMR (400 MHz, DMSO-d₆) δ (ppm) 9.79 (s, 1H), 8.99 (s, 1H), 8.38 (d, J = 8.0 Hz, 1H), 7.97 (brs, 3H), 7.83-7.78(m, 2H), 7.66 (s, 1H), 7.44-7.36 (m, 4H), 7.19 (t, J= 8.0 Hz, 1H), 6.42 (dd, J= 8.0 Hz, J= 1.2 Hz, 1H), 6.18 (brs, 1H), 4.93-4.89 (m, 1H), 4.51 (d, J= 8.8 Hz, 1H), 4.41 (t, J= 8.0 Hz, 1H), 4.28 (s, 1H), 4.05-4.02 (m, 2H), 3.61-3.51 (m, 4H), 3.36-3.26 (m, 4H), 3.15-2.99 (m, 6H), 2.73-2.61 (m, 2H), 2.45 (s, 3H), 2.33-2.22 (m, 2H), 2.13-1.98 (m, 3H), 1.82-1.63 (m, 7H), 1.49-1.45 (m, 2H),1.38 (s, 3H), 1.36 (s, 3H), 1.32-1.21 (m, 10H), 0.93 (s, 9H); UPLC-MS (ESI⁺) calculated for C₅₆H₈₂ClN₁₂O₅S₂ [M+1]⁺: 1101.57, found 1101.62.

The compounds of Table 5 were made using the procedure or modifications to the procedure as exemplified for Example 2.

TABLE 5

| Compound | MW | Observed [M+H]/z | ¹H NMR |
|---|---|---|---|
| 24 | 1115.58 | 1115.53 | δ (ppm) 9.80 (s, 1H), 8.99 (s, 1H), 8.37 (d, J= 8.0 Hz, 1H), 7.97 (brs, 3H), 7.83-7.78(m, 2H), 7.66 (s, 1H), 7.45-7.36 (m, 4H), 7.20 (t, J= 8.0 Hz, 1H), 6.42 (dd, J = 8.0 Hz, J= 1.2 Hz, 1H), 6.18 (brs, 1H), 4.95-4.87 (m, 1H), 4.51 (d, J= 8.8 Hz, 1H), 4.41 (t, J= 8.0 Hz, 1H), 4.28 (s, 1H), 4.05-4.02 (m, 2H), 3.63-3.52 (m, 4H), 3.38-3.26 (m, 4H), 3.15-2.99 (m, 6H), 2.73-2.63 (m, 2H), 2.45 (s, 3H), 2.29-2.22 (m, 2H), 2.13-1.98 (m, 3H), 1.82-1.63 (m, 7H), 1.52-1.43 (m, 2H),1.38 (s, 3H), 1.36 (s, 3H), 1.32-1.21 (m, 12H), 0.93 (s, 9H) |
| 25 | 1059.54 | 1059.51 | δ (ppm) 9.49 (s, 1H), 8.99 (s, 1H), 8.38 (d, J= 8.0 Hz, 1H), 7.95 (brs, 3H), 7.81-7.78 (m, 2H), 7.66 (s, 1H), 7.45-7.34 (m, 4H), 7.15 (t, J= 8.0 Hz, 1H), 6.42 (dd, J = 8.0 Hz, J= 1.2 Hz, 1H), 6.18 (brs, 2H), 4.95-4.88 (m, 1H), 4.51 (d, J= 8.8 Hz, 1H), 4.42 (t, J= 8.0 Hz, 1H), 4.27 (s, 1H), 4.05-4.02 (m, 2H), 3.63-3.57 (m, 2H), 3.33-3.26 (m, 2H), 2.45 (s, 3H), 2.38-2.34 (m, 2H), 2.28-2.17 (m, 1H), 2.16-1.98 (m, 2H), 1.80-1.68 (m, 5H), 1.60-1.56 (m, 2H), 1.53-1.41 (m, 2H), 1.38 (s, 3H), 1.36 (s, 3H), 1.34-1.18 (m, 20H), 0.93 (s, 9H) |

### Example 3

### N1-(Amino-5-(4-Amino-4-Methylpiperidin-1-yl)Pyrazin-2-yl)Thio)-2-Chlorophenyl)-N3-(2-((2-(2,6-Dioxopiperidin-3-yl)-1,3-Dioxoisoindolin-4-yl)Amino)Ethyl)Malonamide

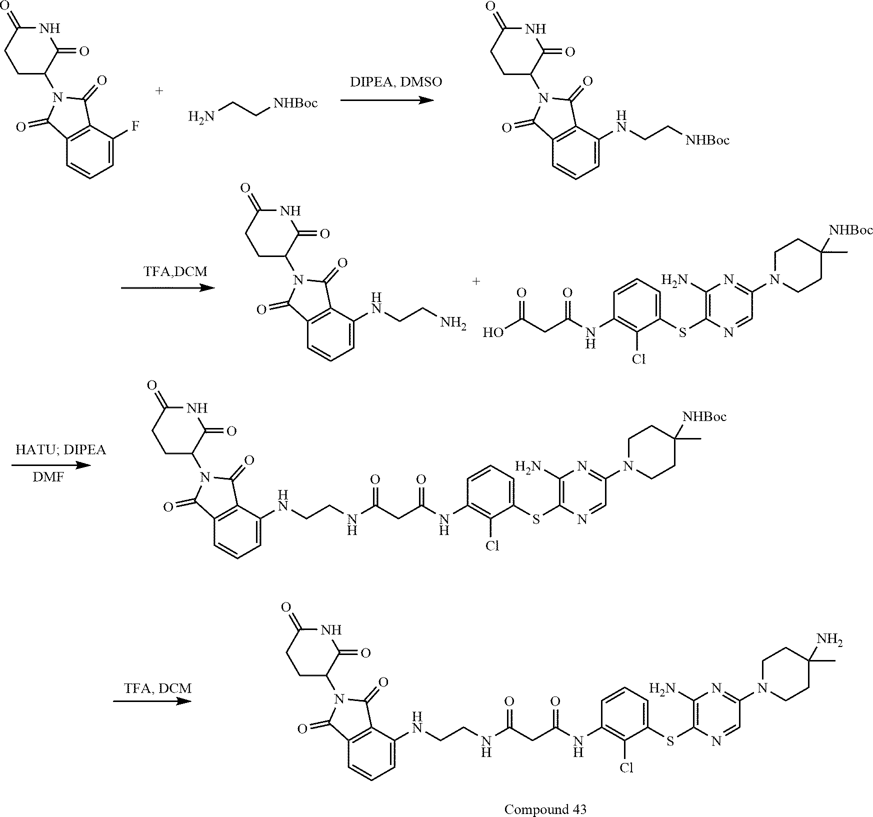

Step 1: To the solution of 2-(2,6-dioxopiperidin-3-yl)-4-fluoroisoindoline-1,3-dione (276 mg, 1.0 mmole) and tert-butyl (2-aminoethyl)carbamate (192 mg, 1.2 mmole, 1.2 eq) in DMSO (4 mL) was added DIPEA (387 mg, 3.0 mmole, 3.0 eq) at rt, then stirred at 90° C. overnight, then purified by HPLC afford tert-butyl (2-((2-(2,6-dioxopiperidin-3-yl)-1,3-dioxoisoindolin-4-yl)amino)ethyl)carbamate (132 mg, 32% yield).

Step 2: To the solution of tert-butyl (2-((2-(2,6-dioxopiperidin-3-yl)-1,3-dioxoisoindolin-4-yl)amino)ethyl)carbamate (132 mg ) in DCM (4 mL) was added TFA ( 1 mL) at 0° C., then stirred at rt for 2h and solvent was removed under vacumm to afford 4-((2-aminoethyl)amino)-2-(2,6-dioxopiperidin-3-yl)isoindoline-1,3-dione as TFA salt.

Step 3: To the solution of 3-((3-((3-amino-5-(4-((tert-butoxycarbonyl)amino)-4-methylpiperidin-1-yl)pyrazin-2-yl)thio)-2-chlorophenyl)amino)-3-oxopropanoic acid ( 55 mg, 0.1 mmole) and 4-((2-aminoethyl)amino)-2-(2,6-dioxopiperidin-3-yl)isoindoline-1,3-dione TFA salt ( 48 mg, 0.11 mmole, 1.1 eq) was added DIPEA ( 65 mg, 0.5 mmole, 5.0 eq) and HATU ( 42 mg, 0.11 mmole, 1.1 eq) at 0° C. The mixture was stirred at ambient temperature for 1 h. After quenched with water (8 mL) and extracted with ethyl acetate (5 mL × 3), the organic layers were washed with brine (15 mL), dried over anhydrous Na₂SO₄, and concentrated under a reduced pressure. The residue was dissolved in DCM (3 mL) was added trifluoracetic acid (1 mL) at 0° C. The reaction was stirred for 30 min, and the solvent was removed in vacuo. The residue was purified by reverse-phase chromatography over a C18 column to yield Compound 43 as a yellow powder (34 mg, 45% yield). UPLC-MS (ESI⁺) calculated for C₃₄H₃₈CIN₁₀O₆S [M+1]⁺: 749.24, found 749.13.

The following compounds of Table 6 were made using the procedure or modifications to the procedure as exemplified for Example 3.

TABLE 6

| Compound | MW | Observed [M+H]/z |
|---|---|---|
| 44 | 777.27 | 777.11 |
| 45 | 805.30 | 805.13 |
| 46 | 819.32 | 819.21 |
| 47 | 833.33 | 833.16 |
| 48 | 861.36 | 861.22 |
| 49 | 889.39 | 889.25 |
| 50 | 851.31 | 851.12 |
| 51 | 881.32 | 881.17 |
| 52 | 749.24 | 749.17 |
| 53 | 777.27 | 777.11 |
| 54 | 805.30 | 805.16 |
| 55 | 833.33 | 833.37 |
| 56 | 881.32 | 881.17 |
| 57 | 909.35 | 909.21 |
| 58 | 891.34 | 891.16 |
| 59 | 859.35 | 859.22 |
| 60 | 845.37 | 845.25 |
| 61 | 786.26 | 786.1 |

### Example 4

### Synthesis of Compound 4 and 5

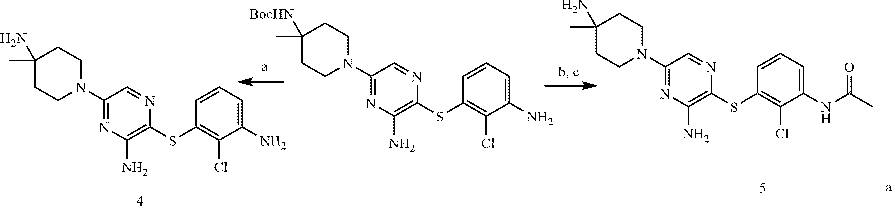

Reagents and conditions: (a) TFA, DCM, rt, 1 h; (b) Acetyl chloride, DIPEA, DCM, 0° C. to RT, 1 h; (c) TFA, DCM, rt, 1h.

3-((3-amino-2-chlorophenyl)thio)-6-(4-amino-4-methylpiperidin-l-yl)pyrazin-2-amine (4). To a solution of tert-butyl (1-(6-amino-5-((3-amino-2-chlorophenyl)thio)pyrazin-2-yl)-4-methylpiperidin-4-yl (60.0 mg, 0.13 mmole) in DCM (2 mL) was added TFA (0.5 mL) at rt. The reaction was stirred for 1 h at rt, and the solvent was removed in vacuo. The residue was purified by reverse-phase chromatography over a C18 column to yield compound 4 as a white-yellow powder. ¹H NMR (400 MHz, DMSO-d₆) δ (ppm) 7.98 (brs, 1H), 7.63 (s, 1H), 6.84 (t, J= 8.0 Hz, 1H), 6.56 (dd, J= 8.0 Hz, J = 1.2 Hz, 1H), 6.10 (brs, 2H); 5.82 (dd, J= 8.0 Hz, J = 1.6 Hz, 1H), 3.86-3.82 (m, 2H), 3.32-3.24 (m, 2H), 1.73-1.69 (m, 4H), 1.37 (s, 3H); UPLC-MS (ESI⁺) calculated for C₁₆H₂₂ClN₆S [M+1]⁺: 365.13, found 365.19.

N-(3-((3-amino-5-(4-amino-4-methylpiperidin-l-yl)pyrazin-2-yl)t′hio)-2-chlorophenyl)acetamide (5). Acetyl chloride (16.2 mg, 0.21 mmol, 1.2 equiv) was added dropwise at 0° C. to a solution of tert-butyl (1-(6-amino-5-((3-amino-2-chlorophenyl)thio)pyrazin-2-yl)-4-methylpiperidin-4-yl (80.0 mg, 0.17 mmol, 1.0 equiv) and DIPEA (65.8 mg, 0.51 mmol, 3.0 equiv) in DCM (3 mL). The reaction mixture was allowed to warm to rt and stirred for 1 h. It was poured onto aq. NaHCO₃ solution (10 mL). After extraction with DCM (10 mL × 2), the combined organic layers were washed with brine, dried over Na₂SO₄ and evaporated under vacuum. The residue was dissolved in DCM (2 mL) and TFA (0.5 mL) was added at rt. The reaction was stirred for 1 h at rt, and the solvent was removed in vacuo. The residue was purified by reverse-phase chromatography over a C18 column to yield 5 as a white-yellow powder. ¹H NMR (400 MHz, DMSO-d₆) δ (ppm) 9.56 (s, 1H), 7.89 (brs,2 H), 7.66 (s, 1H), 7.44 (d, J= 7.6 Hz, 1H), 7.18 (t, J= 8.0 Hz, 1H), 6.43 (dd, J= 8.0 Hz, J= 1.2 Hz, 1H), 6.20 (s, 1H); 4.06-4.02 (m, 2H), 3.32-3.27 (m, 2H), 2.09 (s, 3H), 1.73-1.68 (m, 2H), 1.37 (s, 3H); UPLC-MS (ESI⁺) calculated for C₁₈H₂₄ClN₆OS [M+1]⁺: 407.14, found 407.31.

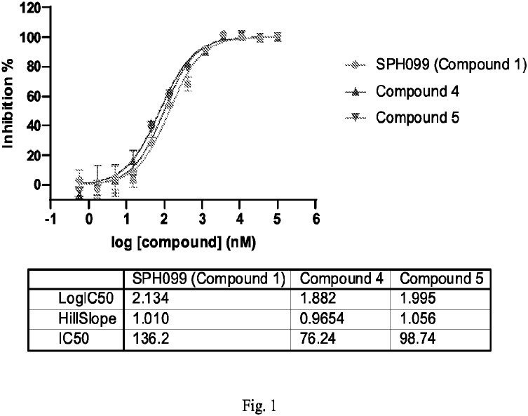

Compound 4 was found to be a potent SHP2 inhibitor with IC₅₀ = 76.2 nM. In the same assay, SHP099 is slightly less potent, with IC₅₀ = 136.2 nM. Compound 5 is also a SHP2 inhibitor with IC₅₀ = 98.7 nM. These data are shown in FIG. 1. The predicted binding model of compound 5 with SHP2 showed the acetyl group extends further into the solvent exposed region making it a suitable site for tethering. The SHP2 allosteric inhibition assay was performed at room temperature in 384-well black polystyrene plate, flat bottom, low flange, nonbinding surface (Corning, cat. no. 3575) using a final reaction volume of 25 µL. Assay buffer conditions: 60 mM HEPES, pH 7.2, 75 mM NaCl, 75 mM KCl, 0.05% P-20, 5 mM DTT,1 mM EDTA. 0.5 nM of SHP2 was incubated with 0.5 µM of bisphosphorylated IRS1 peptide and 100 µM (top concentration 3X dilution) of the inhibitory compounds. After incubation 60 min at RT, 5 µl 200 µM substrate DiFMUP (Invitrogen) was added and incubated another 30 min at RT. The reaction was then quenched by adding 5 µl 160 µM bpV(Phen) (Enzo Life Sciences). The fluorescence signal was detected by CLARIOstar (BMG tech), excitation wavelength was 340 nm and emission wavelength was 450 nm. The inhibitor dose-response curves were analyzed by Graphpad Prism 8.

### Example 5

### Biological Studies

### Cell Lines and Cell Culture

The MV4;11 cell line was purchased from the American Type Culture Collection (ATCC), cultured in Iscove’s modified Dulbecco’s media (IMDM). Esophageal cancer cell line KYSE520 was purchased from DSMZ (Braunschweig, Germany), grown in RPMI 1640 (Invitrogen). All of the cells were supplemented with 10% fetal bovine serum (Invitrogen) at 37° C. in a humidified 5% CO2 incubator.

### Cell Growth Assay

Cell viability was evaluated with a WST-8 assay (Dojindo) following the manufacturer’s instructions. Briefly, cells were seeded in 96-well cell culture plates at a density of 10,000-20,000 cells/well in 200 µL for MV4;11 cell line or 2,000-3,000 for KYSE-520 cell line of culture medium containing serial dilution of testing compounds. After 4 days of treatment, cell growth was measured by a lactate dehydrogenase-based WST-8 assay (Dojindo Molecular Technologies) using a Tecan Infinite M-1000 multimode microplate reader (Tecan US, Morrisville, NC). The WST-8 reagent was added to each well, and cells were incubated for an additional 1-2 h and read at 450 nm. The readings were normalized to the vehicle-treated cells, and the IC₅₀ was calculated by nonlinear regression analysis using the GraphPad Prism 6 software.

### Western Blot Analysis

Western blotting and quantification were performed with regular western blot method and band quantification was performed using ImageJ. Treated cells were lysed by RIPA buffer supplemented with protease and phosphatase inhibitors. The cell lysates were separated by 4-12% SDS-PAGE gels and blotted into PVDF (polyvinylidene difluoride) membranes. Antibodies used in the study are indicated in the Figure legends. The net protein bands and loading controls are calculated by deducting the background from the inverted band value. The final relative quantification values are the ratio of the net band to net loading control.

For the in vitro kinetics studies of SHP2 expression, cancer cells seeded in 6-well plate overnight were treated with the compounds for another 2, 4, 8, 12 and 24 h. The treated cells were harvested and the level of SHP2 protein was examined by blot analysis. GAPDH was used as a loading control.

The SHP2 degradation activity of representative Compounds of the Disclosure are provided in Table 7.

TABLE 7

| Compound | % of SHP2 protein degradation in KYSE520 cell line | | % of SHP2 protein degradation in MV4;11 cell line |
|---|---|---|---|
| | 0.1 µM | 1 µM | 0.1 µM |
| 7 | 4 | 0 | |
| 8 | 9 | 7 | |
| 9 | 13 | 6 | |
| 10 | 0 | 3 | |
| 11 | 0 | 0 | |
| 12 | 20 | 14 | |
| 13 | 81 | 80 | |
| 14 | >95 | 92 | |
| 15 | >95 | >95 | |
| 16 | >95 | 93 | |
| 17 | >95 | >95 | |
| 18 | >95 | >95 | |
| 19 | >95 | >95 | |
| 20 | 80 | >95 | >95 |
| 21 | 32 | 87 | 85 |
| 22 | 53 | >95 | 95 |
| 23 | 85 | >95 | 47 |
| 24 | 80 | >95 | 6 |
| 25 | 62 | 90 | 8 |
| 26 | >95 | >95 | >95 |
| 27 | >95 | >95 | >95 |
| 28 | >95 | >95 | 90 |
| 29 | 0 | 82 | 9 |
| 40 | >95 | >95 | >95 |
| 42 | >95 | >95 | 94 |

### Example 6

### Degradation of SHP2 by Compound 26 (SHP2-D26)

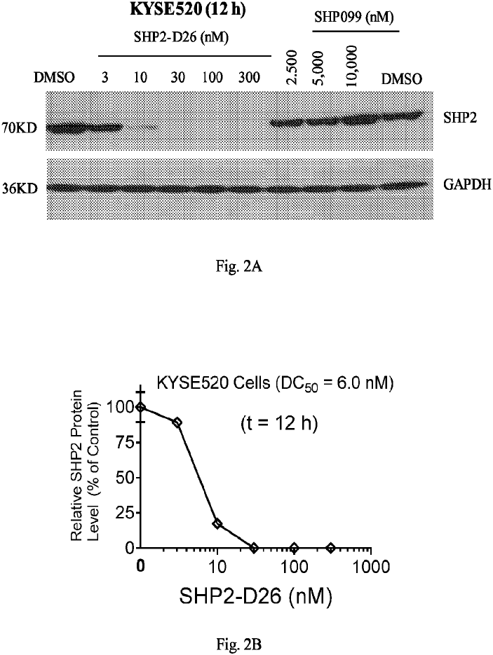

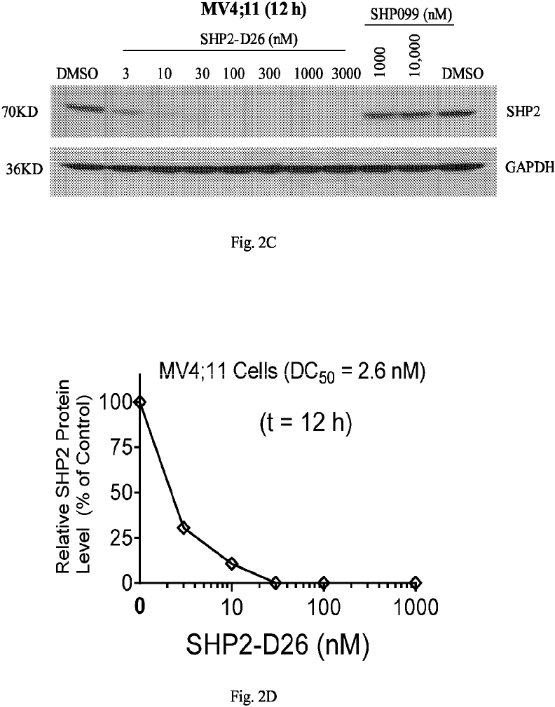

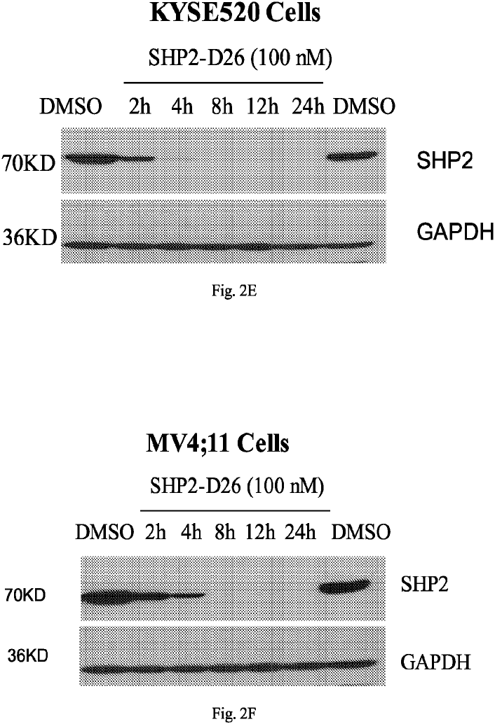

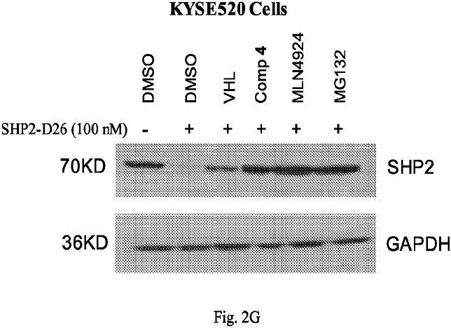

The degradation of SHP2 by Compound 26 (SHP2-D26) in a range of concentrations in KYSE520 and MV4;11 cell lines was examined (FIGS. 2A-D). Western blotting results showed that SHP2-D26 effectively reduces SHP2 protein in a dose-dependent manner. Quantification of the Western blotting data showed that the compound achieves DC₅₀ values (concentration needed to induced targeted protein degradation by 50%) of 6.0 and 2.6 nM in the KYSE520 and MV4;11 cell lines, respectively (FIGS. 2B and 2D).

The kinetics of SHP2-D26 in induction of SHP2 degradation in the KYSE520 and MV4;11 cell lines were evaluated (FIGS. 2E and 2F). In KYSE520 cells, SHP2-D26 at 100 nM reduces the SHP2 protein level within 4 h and achieves essentially complete SHP2 depletion with an 8 h treatment (FIG. 2E). Similar kinetics was observed in the MV4; 11 cell line (FIG. 2F).

Whether SHP2-D26 functions as a bona fide PROTAC degrader in the KYSE520 cell line was examined. A VHL-1 ligand, compound 4 (an SHP2 inhibitor), MLN4924 (an E1 inhibitor), and MG132 (a proteasome inhibitor) all effectively block degradation of SHP2 protein in KYSE520 cells (FIG. 2G). Therefore, our data show that SHP2 degradation induced by SHP2-D26 requires its binding to VHL-1 and SHP2 proteins and is also neddylation- and proteasome-dependent, demonstrating that SHP2-D26 is a bona fide PROTAC SHP2 degrader.

Briefly. Dose-dependent SHP2 degradation in the KYSE520 and MV4;11 cell lines, with SHP099 included as the inhibitor control. Cells were treated with SHP2-D26 for 12 h. SHP2 protein was examined by Western blotting and the protein level was quantified by densitometry and normalized to the corresponding density of the GAPDH protein. (FIGS. 2E and 2F). Degradation kinetics of SHP2-D26 in the KYSE520 and MV4;11 cell lines. Cells were treated with 100 nM of SHP2-D26 for different times. SHP2 protein was examined by Western blotting and the GAPDH protein was used as the loading control. (FIG. 2G) KYSE520 cell line was treated with VHL (10 µM), compound 4 at 10 µM), MLN4924 (0.5 µM) or MG132 (3 µm) for 1 h, then treated with SHP-D26 (0.1 µM) for 1 h. The protein level of SHP2 was examined by Western blotting and the GAPDH protein was used as the loading control.

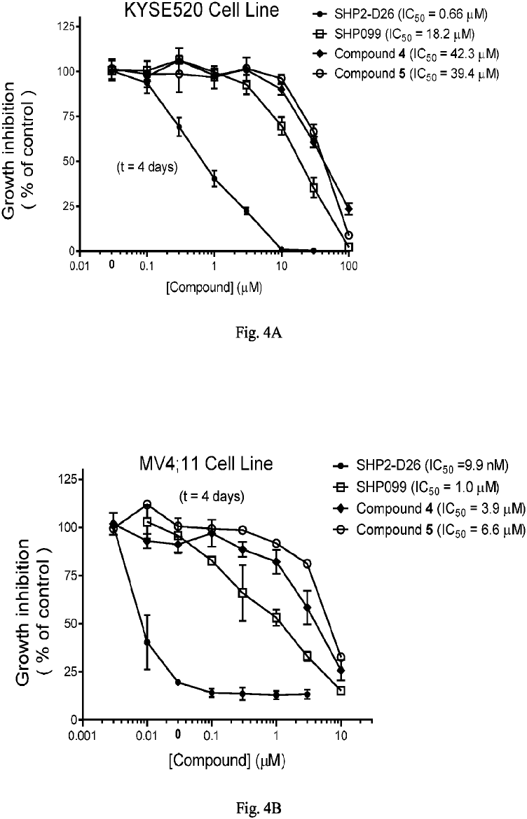

Since SHP2 protein is known to play a role in the MAPK/ERK signaling pathway, the impact of SHP2 degradation on the MAPK/ERK signaling pathway in the KYSE520 and MV4;11 cell lines, with SHP099 included as a control was examined (FIG. 4). Western blotting showed that both SHP2-D26 and SHP099 dose-dependently inhibit phosphorylation of ERK in the KYSE520 and MV4;11 cell lines. However, in both cell lines, SHP2-D26 is more much potent than SHP099 in inhibition of pERK. Specifically, in the KYSE520 cell line, while 100 nM of SHP2-D26 is effective in reducing the level of pERK, >3,000 nM of the SHP2 inhibitor SHP099 is needed to do so. In the MV4;11 cell line, while 100 nM of SHP2-D26 completely inhibits phorsphorylated ERK (pERK), >3,000 nM of the SHP2 inhibitor SHP099 is required to achieve complete inhibition of pERK. These data indicate that SHP2-D26 is >30-times more potent than SHP099 in inhibition of pERK in both cell lines.

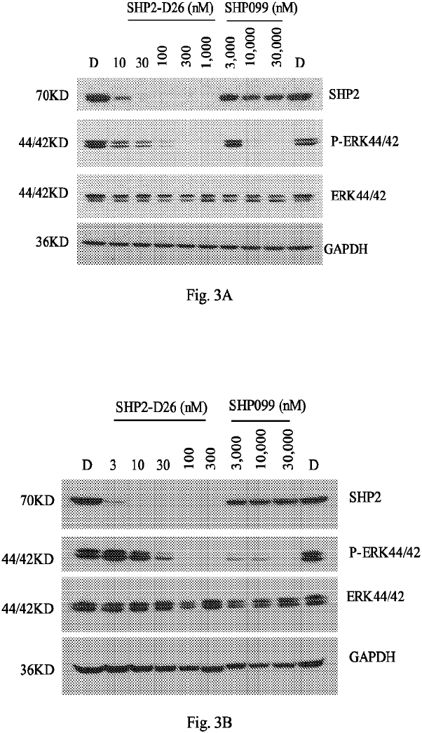

Briefly, KYSE520 or MV4;11 cells were treated as indicated with SHP2-D26 or SHP099 for 48 h. The protein levels of SHP2 (Bethyl Lab. A301-544), ERK (#9102, CST), phospho-ERK (#4370, CST) determined by western blotting. GAPDH was used as a loading control. (FIG. 3A and FIG. 3B).

The cell growth inhibition of SHP2-D26 and three SHP2 inhibitors — SHP099, compound 4, and compound 5 — in the KYSE520 and MV4;11 cell lines. In the KYSE520 cell line, SHP2-D26 achieves IC₅₀ values of 0.66 µM (FIG. 4A). In comparison, SHP099, compound 4, and compound 5 have IC₅₀ values of 18.2 µM, 42.3 µM and 39.4 µM, respectively. SHP2-D26 is surprisingly 28-, 64- and 60-times more potent than SHP099, compound 4, and compound 5 in inhibiting cell growth in the KYSE520 cell line, respectively. In the MV4;11 cell line, SHP2-D26, SHP099, compound 4, and compound 5 have IC₅₀ values of 9.9 nM, 1.0 µM, 3.9 µM and 6.6 pM, respectively, in inhibition of cell growth. SHP2-D26 is surprisingly 100-, >400 and >600-times more potent than SHP099, compound 4, and compound 5, respectively, in cell growth inhibition in the MV4;11 cell line.

Briefly, Cells were treated with indicated doses for 4 days and cell viability was determined by a colorimetric WST-8 assay.

### VI. References

(1) Tartaglia, M.; Mehler, E. L.; Goldberg, R.; Zampino, G.; Brunner, H. G.; Kremer, H.; van der Burgt, I.; Crosby, A. H.; Ion, A.; Jeffery, S.; Kalidas, K.; Patton, M. A.; Kucherlapati, R. S.; Gelb, B. D. Mutations in PTPN11, encoding the protein tyrosine phosphatase SHP-2, cause Noonan syndrome. Nat. Genet. 2001, 29, 465-468.

(2) Kontaridis, M. I.; Swanson, K. D.; David, F. S.; Barford, D.; Neel, B. G. PTPN11 (Shp2) mutations in LEOPARD syndrome have dominant negative, not activating, effects. J. Biol. Chem. 2006, 281, 6785-6792.

(3) Tartaglia, M.; Niemeyer, C. M.; Fragale, A.; Song, X.; Buechner, J.; Jung, A.; Hählen, K.; Hasle, H.; Licht, J. D.; Gelb, B. D. Somatic mutations in PTPN11 in juvenile myelomonocytic leukemia, myelodysplastic syndromes and acute myeloid leukemia. Nat. Genet. 2003, 34, 148-150.

(4) Bentires-Alj, M.; Paez, J. G.; David, F. S.; Keilhack, H.; Halmos, B.; Naoki, K.; Maris, J. M.; Richardson, A.; Bardelli, A.; Sugarbaker, D. J.; Richards, W. G.; Du, J.; Girard, L.; Minna, J. D.; Loh, M. L.; Fisher, D. E.; Velculescu, V. E.; Vogelstein, B.; Meyerson, M.; Sellers, W. R.; Neel, B. G. Activating mutations of the noonan syndrome-associated SHP2/PTPN11 gene in human solid tumors and adult acute myelogenous leukemia. Cancer. Res. 2004, 64, 8816-8820.

(5) Comprehensive genomic characterization defines human glioblastoma genes and core pathways. Nature 2008, 455, 1061-1068.

(6) Chan, G.; Kalaitzidis, D.; Neel, B. G. The tyrosine phosphatase Shp2 (PTPN11) in cancer. Cancer Metastasis Rev. 2008, 27, 179-192.

(7) Miyamoto, D.; Miyamoto, M.; Takahashi, A.; Yomogita, Y.; Higashi, H.; Kondo, S.; Hatakeyama, M. Isolation of a distinct class of gain-of-function SHP-2 mutants with oncogenic RAS-like transforming activity from solid tumors. Oncogene 2008, 27, 3508-3515.

(8) Aceto, N.; Sausgruber, N.; Brinkhaus, H.; Gaidatzis, D.; Martiny-Baron, G.; Mazzarol, G.; Confalonieri, S.; Quarto, M.; Hu, G.; Balwierz, P. J.; Pachkov, M.; Elledge, S. J.; van Nimwegen, E.; Stadler, M. B.; Bentires-Alj, M. Tyrosine phosphatase SHP2 promotes breast cancer progression and maintains tumor-initiating cells via activation of key transcription factors and a positive feedback signaling loop. Nat. Med. 2012, 18, 529-537.

(9) Hanafusa, H.; Torii, S.; Yasunaga, T.; Nishida, E. Sproutyl and Sprouty2 provide a control mechanism for the Ras/MAPK signalling pathway. Nat. Cell Biol. 2002, 4, 850-858.

(10) Agazie, Y. M.; Hayman, M. J. Molecular mechanism for a role of SHP2 in epidermal growth factor receptor signaling. Mol. Cell Biol. 2003, 23, 7875-7886.

(11) Xu, D.; Qu, C. K. Protein tyrosine phosphatases in the JAK/STAT pathway. Front. Biosci. 2008, 13, 4925-4932.

(12) Matozaki, T.; Murata, Y.; Saito, Y.; Okazawa, H.; Ohnishi, H. Protein tyrosine phosphatase SHP-2: a proto-oncogene product that promotes Ras activation. Cancer Sci. 2009, 100, 1786-1793.

(13) Bunda, S.; Burrell, K.; Heir, P.; Zeng, L.; Alamsahebpour, A.; Kano, Y.; Raught, B.; Zhang, Z.-Y.; Zadeh, G.; Ohh, M. Inhibition of SHP2-mediated dephosphorylation of Ras suppresses oncogenesis. Nat. Commun. 2015, 6, 8859.

(14) Chemnitz, J. M.; Parry, R. V.; Nichols, K. E.; June, C. H.; Riley, J. L. SHP-1 and SHP-2 associate with immunoreceptor tyrosine-based switch motif of programmed death 1 upon primary human T cell stimulation, but only receptor ligation prevents T cell activation. J. Immunol. 2004, 173, 945-954.

(15) Li, J.; Jie, H. B.; Lei, Y.; Gildener-Leapman, N.; Trivedi, S.; Green, T.; Kane, L. P.; Ferris, R. L. PD-1/SHP-2 inhibits Tc1/Th1 phenotypic responses and the activation of T cells in the tumor microenvironment. Cancer Res. 2015, 75, 508-518.

(16) Hui, E.; Cheung, J.; Zhu, J.; Su, X.; Taylor, M. J.; Wallweber, H. A.; Sasmal, D. K.; Huang, J.; Kim, J. M.; Mellman, I.; Vale, R. D. T cell costimulatory receptor CD28 is a primary target for PD-1-mediated inhibition. Science 2017, 355, 1428-1433.

(17) Scott, L. M.; Lawrence, H. R.; Sebti, S. M.; Lawrence, N. J.; Wu, J. Targeting protein tyrosine phosphatases for anticancer drug discovery. Curr. Pharm. Des. 2010, 16, 1843-1862.

(18) Butterworth, S.; Overduin, M.; Barr, A. J. Targeting protein tyrosine phosphatase SHP2 for therapeutic intervention. Future Med. Chem. 2014, 6, 1423-1437.

(19) Chen, L.; Sung, S. S.; Yip, M. L.; Lawrence, H. R.; Ren, Y.; Guida, W. C.; Sebti, S. M.; Lawrence, N. J.; Wu, J. Discovery of a novel shp2 protein tyrosine phosphatase inhibitor. Mol. Pharmacol. 2006, 70, 562-570.

(20) Hellmuth, K.; Grosskopf, S.; Lum, C. T.; Würtele, M.; Röder, N.; von Kries, J. P.; Rosario, M.; Rademann, J.; Birchmeier, W. Specific inhibitors of the protein tyrosine phosphatase Shp2 identified by high-throughput docking. Proc. Natl. Acad. Sci. U.S.A. 2008, 105, 7275-7280.

(21) Lawrence, H. R.; Pireddu, R.; Chen, L.; Luo, Y.; Sung, S.-S.; Szymanski, A. M.; Yip, M. L. R.; Guida, W. C.; Sebti, S. M.; Wu, J.; Lawrence, N. J. Inhibitors of Src homology-2 domain containing protein tyrosine phosphatase-2 (Shp2) based on oxindole scaffolds. J. Med. Chem. 2008, 51, 4948-4956.

(22) Zhang, X.; He, Y.; Liu, S.; Yu, Z.; Jiang, Z. X.; Yang, Z.; Dong, Y.; Nabinger, S. C.; Wu, L.; Gunawan, A. M.; Wang, L.; Chan, R. J.; Zhang, Z. Y. Salicylic acid based small molecule inhibitor for the oncogenic Src homology-2 domain containing protein tyrosine phosphatase-2 (SHP2). J. Med. Chem. 2010, 53, 2482-2493.

(23) Liu, W.; Yu, B.; Xu, G.; Xu, W.-R.; Loh, M. L.; Tang, L.-D.; Qu, C.-K. Identification of cryptotanshinone as an inhibitor of oncogenic protein tyrosine phosphatase SHP2 (PTPN11). J. Med. Chem. 2013, 56, 7212-7221.

(24) Zeng, L.-F.; Zhang, R.-Y.; Yu, Z.-H.; Li, S.; Wu, L.; Gunawan, A. M.; Lane, B. S.; Mali, R. S.; Li, X.; Chan, R. J.; Kapur, R.; Wells, C. D.; Zhang, Z.-Y. Therapeutic potential of targeting the oncogenic SHP2 phosphatase. J. Med. Chem. 2014, 57, 6594-6609.

(25) Chio, C. M.; Lim, C. S.; Bishop, A. C. Targeting a cryptic allosteric site for selective inhibition of the oncogenic protein tyrosine phosphatase Shp2. Biochemistry 2015, 54, 497-504.

(26) Chen, Y. N.; LaMarche, M. J.; Chan, H. M.; Fekkes, P.; Garcia-Fortanet, J.; Acker, M. G.; Antonakos, B.; Chen, C. H.; Chen, Z.; Cooke, V. G.; Dobson, J. R.; Deng, Z.; Fei, F.; Firestone, B.; Fodor, M.; Fridrich, C.; Gao, H.; Grunenfelder, D.; Hao, H. X.; Jacob, J.; Ho, S.; Hsiao, K.; Kang, Z. B.; Karki, R.; Kato, M.; Larrow, J.; La Bonte, L. R.; Lenoir, F.; Liu, G.; Liu, S.; Majumdar, D.; Meyer, M. J.; Palermo, M.; Perez, L.; Pu, M.; Price, E.; Quinn, C.; Shakya, S.; Shultz, M. D.; Slisz, J.; Venkatesan, K.; Wang, P.; Warmuth, M.; Williams, S.; Yang, G.; Yuan, J.; Zhang, J. H.; Zhu, P.; Ramsey, T.; Keen, N. J.; Sellers, W. R.; Stams, T.; Fortin, P. D. Allosteric inhibition of SHP2 phosphatase inhibits cancers driven by receptor tyrosine kinases. Nature 2016, 535, 148-152.

(27) Garcia Fortanet, J.; Chen, C. H.; Chen, Y. N.; Chen, Z.; Deng, Z.; Firestone, B.; Fekkes, P.; Fodor, M.; Fortin, P. D.; Fridrich, C.; Grunenfelder, D.; Ho, S.; Kang, Z. B.; Karki, R.; Kato, M.; Keen, N.; LaBonte, L. R.; Larrow, J.; Lenoir, F.; Liu, G.; Liu, S.; Lombardo, F.; Majumdar, D.; Meyer, M. J.; Palermo, M.; Perez, L.; Pu, M.; Ramsey, T.; Sellers, W. R.; Shultz, M. D.; Stams, T.; Towler, C.; Wang, P.; Williams, S. L.; Zhang, J. H.; LaMarche, M. J. Allosteric inhibition of SHP2: identification of a potent, selective, and orally efficacious phosphatase inhibitor. J. Med. Chem. 2016, 59, 7773-7782.

(28) Xie, J.; Si, X.; Gu, S.; Wang, M.; Shen, J.; Li, H.; Shen, J.; Li, D.; Fang, Y.; Liu, C.; Zhu, J. Allosteric inhibitors of SHP2 with therapeutic potential for cancer treatment. J. Med. Chem. 2017, 60, 10205-10219.

(29) Koltun, E. S.; Aay, N.; Buckl, A.; Jogalekar, A. S.; Kiss, G.; Marquez, A.; Mellem, K. T.; Mordec, K.; Saldajeno-Concar, M.; Semko, C. M. RMC-4550, an allosteric inhibitor of SHP2: synthesis, structure, and anti-tumor activity. Presented at the Annual Meeting of the American Association for Cancer Research (AACR). Chicago,IL, Philadelphia (PA): 2018; Abstract 4878.

(30) Bagdanoff, J. T.; Chen, Z.; Acker, M.; Chen, Y. N.; Chan, H.; Dore, M.; Firestone, B.; Fodor, M.; Fortanet, J.; Hentemann, M.; Kato, M.; Koenig, R.; LaBonte, L. R.; Liu, S.; Mohseni, M.; Ntaganda, R.; Sarver, P.; Smith, T.; Sendzik, M.; Stams, T.; Spence, S.; Towler, C.; Wang, H.; Wang, P.; Williams, S. L.; LaMarche, M. J. Optimization of fused bicyclic allosteric SHP2 inhibitors. J. Med. Chem. 2019, 62, 1781-1792.

(31) Sarver, P.; Acker, M.; Bagdanoff, J. T.; Chen, Z.; Chen, Y. N.; Chan, H.; Firestone, B.; Fodor, M.; Fortanet, J.; Hao, H.; Hentemann, M.; Kato, M.; Koenig, R.; LaBonte, L. R.; Liu, G.; Liu, S.; Liu, C.; McNeill, E.; Mohseni, M.; Sendzik, M.; Stams, T.; Spence, S.; Tamez, V.; Tichkule, R.; Towler, C.; Wang, H.; Wang, P.; Williams, S. L.; Yu, B.; LaMarche, M. J. 6-Amino-3-methylpyrimidinones as potent, selective, and orally efficacious SHP2 inhibitors. J. Med. Chem. 2019, 62, 1793-1802.

(32) Mainardi, S.; Mulero-Sánchez, A.; Prahallad, A.; Germano, G.; Bosma, A.; Krimpenfort, P.; Lieftink, C.; Steinberg, J. D.; de Wit, N.; Gonçalves-Ribeiro, S.; Nadal, E.; Bardelli, A.; Villanueva, A.; Bernards, R. SHP2 is required for growth of KRAS-mutant non-small-cell lung cancer in vivo. Nat. Med. 2018, 24, 961-967.

(33) Ruess, D. A.; Heynen, G. J.; Ciecielski, K. J.; Ai, J.; Berninger, A.; Kabacaoglu, D.; Görgülü, K.; Dantes, Z.; Wörmann, S. M.; Diakopoulos, K. N.; Karpathaki, A. F.; Kowalska, M.; Kaya-Aksoy, E.; Song, L.; van der Laan, E. A. Z.; L6pez-Alberca, M. P.; Nazaré, M.; Reichert, M.; Saur, D.; Erkan, M. M.; Hopt, U. T.; Sainz, B.; Birchmeier, W.; Schmid, R. M.; Lesina, M.; Algül, H. Mutant KRAS-driven cancers depend on PTPN11/SHP2 phosphatase. Nat. Med. 2018, 24, 954-960.

(34) Sakamoto, K. M.; Kim, K. B.; Kumagai, A.; Mercurio, F.; Crews, C. M.; Deshaies, R. J. Protacs: chimeric molecules that target proteins to the Skp1-Cullin-F box complex for ubiquitination and degradation. Proc. Natl. Acad. Sci. U.S.A. 2001, 98, 8554-8559.

(35) Toure, M.; Crews, C. M. Small-molecule PROTACS: new approaches to protein degradation. Angew. Chem., Int. Ed. 2016, 55, 1966-1973.

(36) Lai, A. C.; Crews, C. M. Induced protein degradation: an emerging drug discovery paradigm. Nat. Rev. Drug Discov. 2017, 16, 101-114.

(37) Pettersson, M.; Crews, C. M. Proteolysis targeting chimeras (PROTACs) -past, present and future. Drug Discov. Today Technol. 2019, 31, 15-27.

(38) Zengerle, M.; Chan, K. H.; Ciulli, A. Selective small molecule induced degradation of the BET bromodomain protein BRD4. ACS Chem. Biol. 2015, 10, 1770-1777.

(39) Burslem, G. M.; Song, J.; Chen, X.; Hines, J.; Crews, C. M. Enhancing antiproliferative activity and selectivity of a FLT-3 inhibitor by proteolysis targeting chimera conversion. J. Am. Chem. Soc. 2018, 140, 16428-16432.

(40) McCoull, W.; Cheung, T.; Anderson, E.; Barton, P.; Burgess, J.; Byth, K.; Cao, Q.; Castaldi, M. P.; Chen, H.; Chiarparin, E.; Carbajo, R. J.; Code, E.; Cowan, S.; Davey, P. R.; Ferguson, A. D.; Fillery, S.; Fuller, N. O.; Gao, N.; Hargreaves, D.; Howard, M. R.; Hu, J.; Kawatkar, A.; Kemmitt, P. D.; Leo, E.; Molina, D. M.; O′Connell, N.; Petteruti, P.; Rasmusson, T.; Raubo, P.; Rawlins, P. B.; Ricchiuto, P.; Robb, G. R.; Schenone, M.; Waring, M. J.; Zinda, M.; Fawell, S.; Wilson, D. M. Development of a novel B-cell lymphoma 6 (BCL6) PROTAC to provide insight into small molecule targeting of BCL6. ACS Chem. Biol. 2018, 13, 3131-3141.

(41) Powell, C. E.; Gao, Y.; Tan, L.; Donovan, K. A.; Nowak, R. P.; Loehr, A.; Bahcall, M.; Fischer, E. S.; Jänne, P. A.; George, R. E.; Gray, N. S. Chemically induced degradation of anaplastic lymphoma kinase (ALK). J. Med. Chem. 2018, 61, 4249-4255.

(42) Qin, C.; Hu, Y.; Zhou, B.; Fernandez-Salas, E.; Yang, C.-Y.; Liu, L.; McEachern, D.; Przybranowski, S.; Wang, M.; Stuckey, J.; Meagher, J.; Bai, L.; Chen, Z.; Lin, M.; Yang, J.; Ziazadeh, D. N.; Xu, F.; Hu, J.; Xiang, W.; Huang, L.; Li, S.; Wen, B.; Sun, D.; Wang, S. Discovery of QCA570 as an exceptionally potent and efficacious proteolysis targeting chimera (PROTAC) degrader of the bromodomain and extra-terminal (BET) proteins capable of inducing complete and durable tumor regression. J. Med. Chem. 2018, 61, 6685-6704.

(43) Sun, Y.; Zhao, X.; Ding, N.; Gao, H.; Wu, Y.; Yang, Y.; Zhao, M.; Hwang, J.; Song, Y.; Liu, W.; Rao, Y. PROTAC-induced BTK degradation as a novel therapy for mutated BTK C481S induced ibrutinib-resistant B-cell malignancies. Cell Res. 2018, 28, 779-781.

(44) Jiang, B.; Wang, E. S.; Donovan, K. A.; Liang, Y.; Fischer, E. S.; Zhang, T.; Gray, N. S. Development of dual and selective degraders of cyclin-dependent kinases 4 and 6. Angew. Chem., Int. Ed. 2019, 58, 6321-6326.

(45) Zhou, H.; Bai, L.; Xu, R.; Zhao, Y.; Chen, J.; McEachem, D.; Chinnaswamy, K.; Wen, B.; Dai, L.; Kumar, P.; Yang, C.-Y.; Liu, Z.; Wang, M.; Liu, L.; Meagher, J. L.; Yi, H.; Sun, D.; Stuckey, J. A.; Wang, S. Structure-based discovery of SD-36 as a potent, selective, and efficacious PROTAC degrader of STAT3 protein. J. Med. Chem. 2019, 62, 11280-11300.

(46) Mullard, A. Arvinas’s PROTACs pass first safety and PK analysis. Nat. Rev. Drug Discov. 2019, 18, 895.

(47) Potjewyd, F.; Turner, A. W.; Beri, J.; Rectenwald, J. M.; Norris-Drouin, J. L.; Cholensky, S. H.; Margolis, D. M.; Pearce, K. H.; Herring, L. E.; James, L. I. Degradation of polycomb repressive complex 2 with an EED-targeted bivalent chemical degrader. Cell Chem. Biol. 2020, 27, 47-56.e15.

(48) Khan, S.; Zhang, X.; Lv, D.; Zhang, Q.; He, Y.; Zhang, P.; Liu, X.; Thummuri, D.; Yuan, Y.; Wiegand, J. S.; Pei, J.; Zhang, W.; Sharma, A.; McCurdy, C. R.; Kuruvilla, V. M.; Baran, N.; Ferrando, A. A.; Kim, Y.-m.; Rogojina, A.; Houghton, P. J.; Huang, G.; Hromas, R.; Konopleva, M.; Zheng, G.; Zhou, D. A selective BCL-XL PROTAC degrader achieves safe and potent antitumor activity. Nat. Med. 2019, 25, 1938-1947.

(49) Wei, J.; Hu, J.; Wang, L.; Xie, L.; Jin, M. S.; Chen, X.; Liu, J.; Jin, J. Discovery of a first-in-class mitogen-activated protein kinase kinase ½ degrader. J. Med. Chem. 2019, 62, 10897-10911.

(50) Zhao, Q.; Ren, C.; Liu, L.; Chen, J.; Shao, Y.; Sun, N.; Sun, R.; Kong, Y.; Ding, X.; Zhang, X.; Xu, Y.; Yang, B.; Yin, Q.; Yang, X.; Jiang, B. Discovery of SIAIS178 as an effective BCR-ABL degrader by recruiting von Hippel-Lindau (VHL) E3 ubiquitin ligase. J. Med. Chem. 2019, 62, 9281-9298.

(51) Tovell, H.; Testa, A.; Zhou, H.; Shpiro, N.; Crafter, C.; Ciulli, A.; Alessi, D. R. Design and characterization of SGK3-PROTAC1, an isoform specific SGK3 kinase PROTAC degrader. ACS Chem. Biol. 2019, 14, 2024-2034.

(52) Famaby, W.; Koegl, M.; Roy, M. J.; Whitworth, C.; Diers, E.; Trainor, N.; Zollman, D.; Steurer, S.; Karolyi-Oezguer, J.; Riedmueller, C.; Gmaschitz, T.; Wachter, J.; Dank, C.; Galant, M.; Sharps, B.; Rumpel, K.; Traxler, E.; Gerstberger, T.; Schnitzer, R.; Petermann, O.; Greb, P.; Weinstabl, H.; Bader, G.; Zoephel, A.; Weiss-Puxbaum, A.; Ehrenhöfer-Wölfer, K.; Wöhrle, S.; Boehmelt, G.; Rinnenthal, J.; Amhof, H.; Wiechens, N.; Wu, M.-Y.; Owen-Hughes, T.; Ettmayer, P.; Pearson, M.; McConnell, D. B.; Ciulli, A. BAF complex vulnerabilities in cancer demonstrated via structure-based PROTAC design. Nat. Chem. Biol. 2019, 15, 672-680.

(53) Zoppi, V.; Hughes, S. J.; Maniaci, C.; Testa, A.; Gmaschitz, T.; Wieshofer, C.; Koegl, M.; Riching, K. M.; Daniels, D. L.; Spallarossa, A.; Ciulli, A. Iterative design and optimization of initially inactive proteolysis targeting chimeras (PROTACs) identify VZ185 as a potent, fast, and selective von Hippel-Lindau (VHL) based dual degrader probe of BRD9 and BRD7. J. Med. Chem. 2019, 62, 699-726.

(54) Gechijian, L. N.; Buckley, D. L.; Lawlor, M. A.; Reyes, J. M.; Paulk, J.; Ott, C. J.; Winter, G. E.; Erb, M. A.; Scott, T. G.; Xu, M.; Seo, H.-S.; Dhe-Paganon, S.; Kwiatkowski, N. P.; Perry, J. A.; Qi, J.; Gray, N. S.; Bradner, J. E. Functional TRIM24 degrader via conjugation of ineffectual bromodomain and VHL ligands. Nat. Chem. Biol. 2018, 14, 405-412.

(55) Crew, A. P.; Raina, K.; Dong, H.; Qian, Y.; Wang, J.; Vigil, D.; Serebrenik, Y. V.; Hamman, B. D.; Morgan, A.; Ferraro, C.; Siu, K.; Neklesa, T. K.; Winkler, J. D.; Coleman, K. G.; Crews, C. M. Identification and characterization of von Hippel-Lindau-recruiting proteolysis targeting chimeras (PROTACs) of TANK-binding kinase 1. J. Med. Chem. 2018, 61, 583-598.

(56) Han, X.; Zhao, L.; Xiang, W.; Qin, C.; Miao, B.; Xu, T.; Wang, M.; Yang, C.-Y.; Chinnaswamy, K.; Stuckey, J.; Wang, S. Discovery of highly potent and efficient PROTAC degraders of androgen receptor (AR) by employing weak binding affinity VHL E3 ligase ligands. J. Med. Chem. 2019, 62, 11218-11231.

(57) Wurz, R. P.; Dellamaggiore, K.; Dou, H.; Javier, N.; Lo, M.-C.; McCarter, J. D.; Mohl, D.; Sastri, C.; Lipford, J. R.; Cee, V. J. A “click chemistry platform” for the rapid synthesis of bispecific molecules for inducing protein degradation. J. Med. Chem. 2018, 61, 453-461.

(58) Han, X.; Wang, C.; Qin, C.; Xiang, W.; Fernandez-Salas, E.; Yang, C.-Y.; Wang, M.; Zhao, L.; Xu, T.; Chinnaswamy, K.; Delproposto, J.; Stuckey, J.; Wang, S. Discovery of ARD-69 as a highly potent proteolysis targeting chimera (PROTAC) degrader of androgen receptor (AR) for the treatment of prostate cancer. J. Med. Chem. 2019, 62, 941-964.

(59) Hu, J.; Hu, B.; Wang, M.; Xu, F.; Miao, B.; Yang, C.-Y.; Wang, M.; Liu, Z.; Hayes, D. F.; Chinnaswamy, K.; Delproposto, J.; Stuckey, J.; Wang, S. Discovery of ERD-308 as a highly potent proteolysis targeting chimera (PROTAC) degrader of estrogen receptor (ER). J. Med. Chem. 2019, 62, 1420-1442.

(60) Raina, K.; Lu, J.; Qian, Y.; Altieri, M.; Gordon, D.; Rossi, A. M.; Wang, J.; Chen, X.; Dong, H.; Siu, K.; Winkler, J. D.; Crew, A. P.; Crews, C. M.; Coleman, K. G. PROTAC-induced BET protein degradation as a therapy for castration-resistant prostate cancer. Proc. Natl. Acad. Sci. U.S.A. 2016, 113, 7124-7129.

(61) Beutner, G. L.; Young, I. S.; Davies, M. L.; Hickey, M. R.; Park, H.; Stevens, J. M.; Ye, Q. TCFH-NMI: direct cccess to N-acyl imidazoliums for challenging amide bond formations. Org. Lett. 2018, 20, 4218-4222.

(62) Fodor, M.; Price, E.; Wang, P.; Lu, H.; Argintaru, A.; Chen, Z.; Glick, M.; Hao, H.-X.; Kato, M.; Koenig, R.; LaRochelle, J. R.; Liu, G.; McNeill, E.; Majumdar, D.; Nishiguchi, G. A.; Perez, L. B.; Paris, G.; Quinn, C. M.; Ramsey, T.; Sendzik, M.; Shultz, M. D.; Williams, S. L.; Stams, T.; Blacklow, S. C.; Acker, M. G.; LaMarche, M. J. Dual allosteric inhibition of SHP2 phosphatase. ACS Chem. Biol. 2018, 13, 647-656.

It is to be understood that the foregoing embodiments and exemplifications are not intended to be limiting in any respect to the scope of the disclosure, and that the claims presented herein are intended to encompass all embodiments and exemplifications whether or not explicitly presented herein

All patents and publications cited herein are fully incorporated by reference in their entirety.

What is claimed is:

1. A compound of Formula I: 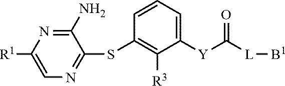
or a pharmaceutically acceptable salt or solvate thereof, wherein
R¹ is selected from the group consisting of 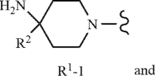 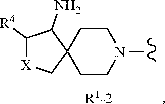
R² is selected from the group consisting of C₁-₄ alkyl and C₃-C₆ cycloalkyl;
R³ is selected from the group consisting of halo and C₁-C₃ haloalkyl;
R⁴ is selected from the group consisting of hydrogen and C₁-₄ alkyl;
X is selected from the group consisting of —O— and —CH₂—;
Y is selected from the group consisting of —NH— and 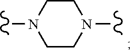
L is —J¹—J²—J³—J⁴—;
J¹ is —(CH₂)ₘ—;
m is 1, 2, 3, or 4;
J² is selected from the group consisting of 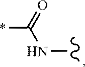 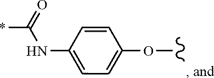 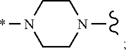
wherein the bond marked with an “*” is attached to J¹; or
J² is absent;
J³ is selected from the group consisting of —(CH₂)ₙ— and —(CH₂CH₂O)ₒ—;
n is 1, 2, 3, 4, 5, 6, 7, 8, 9, 10, 11, 12, 13, 14, 15, or 16;
o is 1, 2, 3, 4, 5, 6, 7, or 8;
J⁴ is selected from the group consisting of —(CH₂)ₚ—, —O—, —N(H)—, —C═C—, 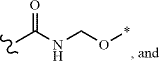 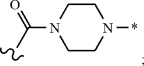
wherein the bond marked with an “*” is attached to B¹;
p is 0, 1, 2, or 3;
with the proviso that J⁴ is —(CH₂)ₚ— and p is 1, 2, or 3, when B¹ is B¹-1;
B¹ is selected from the group consisting of 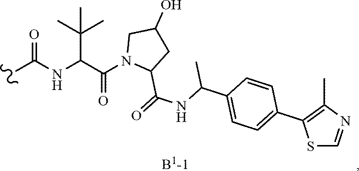 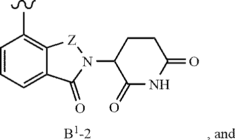 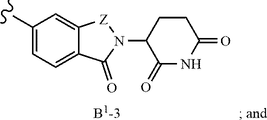
Z is selected from the group consisting of —CH₂ and —C(═O)—.

2. The compound of claim 1, or a pharmaceutically acceptable salt or solvate thereof, wherein R¹ is R¹-1.

3. The compound of claims 1 or 2, or a pharmaceutically acceptable salt or solvate thereof, wherein R² is selected from the group consisting of methyl and ethyl.

4. The compound of claim 1, or a pharmaceutically acceptable salt or solvate thereof, wherein R¹ is R¹-2, R⁴ is hydrogen, and X is —CH₂—.

5. The compound of any one of claims 1-4, or a pharmaceutically acceptable salt or solvate thereof, wherein R³ is —Cl.

6. The compound of any one of claims 1-5, or a pharmaceutically acceptable salt or solvate thereof, wherein B¹ is: 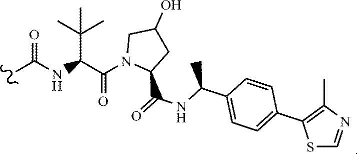
.

7. The compound of any one of claims 1-5, or a pharmaceutically acceptable salt or solvate thereof, wherein B¹ is B¹-2.

8. The compound of claim 7, or a pharmaceutically acceptable salt or solvate thereof, wherein Z is —C(═O)—.

9. The compound of any one of claims 1-5, or a pharmaceutically acceptable salt or solvate thereof, wherein B¹ is B¹-3.

10. The compound of claim 9, or a pharmaceutically acceptable salt or solvate thereof, wherein Z is —C(═O)—.

11. The compound of any one of claims 1-5, or a pharmaceutically acceptable salt or solvate thereof, wherein:
L is selected from the group consisting of: 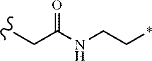 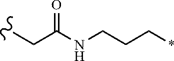 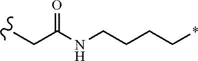 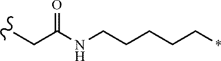 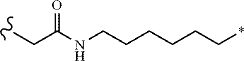 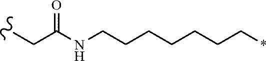 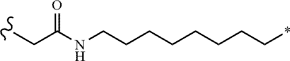 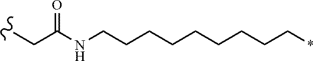 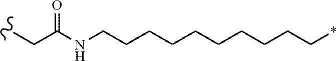 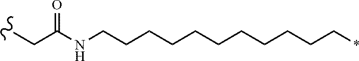 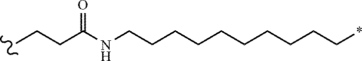 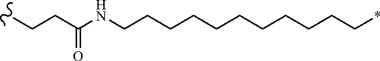 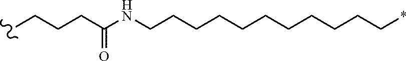 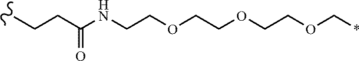 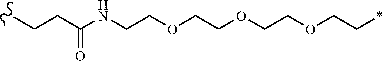 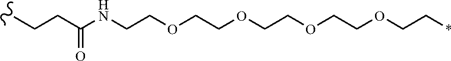 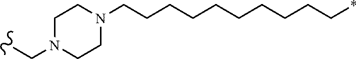 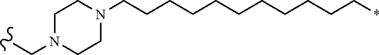 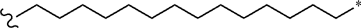 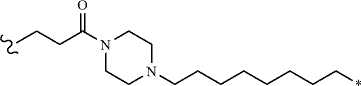 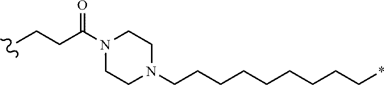 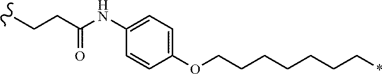 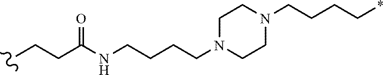
wherein the bond marked with an “*” is attached to B¹; and
B¹ is B¹-1.

12. The compound of any one of claims 1-5, or a pharmaceutically acceptable salt or solvate thereof, wherein:
L is selected from the group consisting of: 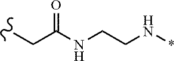 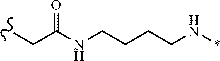 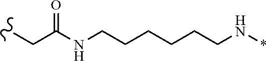 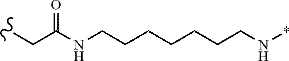 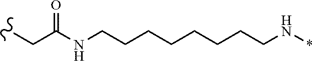 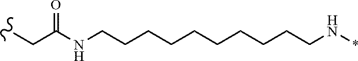 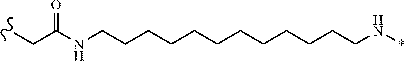 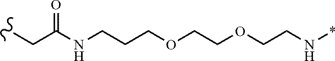 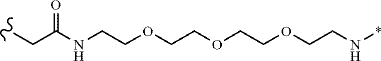 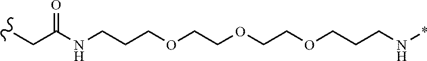 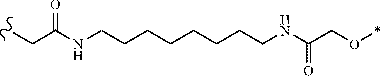 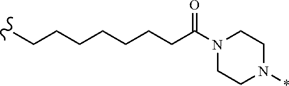 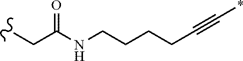
wherein the bond marked with an “*” is attached to B¹; and
B¹ is selected from the group consisting of B¹-2 and B¹-3.

13. The compound of any one of claims 1-12, a pharmaceutically acceptable salt or solvate thereof, wherein Y is —NH—.

14. The compound of claim 1, or a pharmaceutically acceptable salt or solvate thereof, which is any one or more of the compounds of Table 1.

15. A pharmaceutical composition comprising the compound of any one of claims 1-14, or a pharmaceutically acceptable salt or solvate thereof, and a pharmaceutically acceptable excipient.

16. A method of treating cancer in a subject in need thereof, the method comprising administering to the subject a therapeutically effective amount of the compound of any one of claims 1-14, or a pharmaceutically acceptable salt or solvate thereof.

17. The pharmaceutical composition of claim 15 for use in treating cancer.

18. A compound of any one of claims 1-14, or a pharmaceutically acceptable salt or solvate thereof, for use in treating of cancer.

19. Use of a compound of any one of claims 1-14, or a pharmaceutically acceptable salt or solvate thereof, for the manufacture of a medicament for treatment of cancer.

20. A method of reducing SHP2 protein within a cell of a subject in need thereof, the method comprising administering to the subject a compound of any one of claims 1-14, or a pharmaceutically acceptable salt or solvate thereof.

21. A kit comprising the compound of any one of claims 1-14, or a pharmaceutically acceptable salt or solvate thereof, and instructions for administering the compound, or a pharmaceutically acceptable salt or solvate thereof, to a subject having cancer.
